SEQUENCE LISTING (4 sequences)

SEQ ID NO: 1 - DNA, 46 bp, synthetic construct
  misc_feature 1..46: note = Synthetic: 5' read1 adapter strand (FIG. 3) of an exemplary standard Y-shaped adapter
  source 1..46: mol_type = other DNA
gatctacactctttccctacacgacgctcttccgatctcgatgtgt

SEQ ID NO: 2 - DNA, 41 bp, synthetic construct
  misc_feature 1..41: note = Synthetic: 3' read2 adapter strand (FIG. 3) of an exemplary standard Y-shaped adapter
  source 1..41: mol_type = other DNA
cacatcgagatcggaagagcacacgtctgaactccagtcac

SEQ ID NO: 3 - DNA, 42 bp, synthetic construct
  misc_feature 1..42: note = Synthetic: 5' read2 adapter strand (FIG. 3) of an exemplary disclosed Y-shaped adapter
  source 1..42: mol_type = other DNA
gtgactggagttcagacgtgtgctcttccgatctcgatgtgt

SEQ ID NO: 4 - DNA, 45 bp, synthetic construct
  misc_feature 1..45: note = Synthetic: 3' read1 adapter strand (FIG. 3) of an exemplary disclosed Y-shaped adapter
  source 1..45: mol_type = other DNA
cacatcgagatcggaagagcgtcgtgtagggaaagagtgtagatc

(19) United States
(12) Patent Application Publication
(10) Pub. No.: US 2026/0347930 A1
(43) Pub. Date: Nov. 5, 2026

(54) METHODS FOR HIGH QUALITY AND HIGH ACCURACY METHYLATION SEQUENCING

(71) Applicant: GUARDANT HEALTH, INC., Palo Alto, CA (US)

(72) Inventor: Andrew KENNEDY, San Diego, CA (US)

(21) Appl. No.: 19/713,993

(22) Filed: Jun. 19, 2026

Related U.S. Application Data
(63) Continuation of application No. PCT/US2024/061535, filed on Dec. 20, 2024.
(60) Provisional application No. 63/613,592, filed on Dec. 21, 2023.

(51) Int. Cl. C12Q 1/6869; C12N 9/10; C12N 9/78; C12P 19/34; C12Q 1/6806; C12Q 1/6855; C12Q 1/6886

(52) U.S. Cl. CPC C12Q 1/6869; C12N 9/1007; C12N 9/78; C12P 19/34; C12Q 1/6806; C12Q 1/6855; C12Q 1/6886; C12Y 201/01037; C12Y 305/04001

(57) ABSTRACT

Provided herein are methods and compositions for calibrating one or more base calling metrics in a sequencing by synthesis reaction, and for calling at least a portion of nucleobases in a converted region of a DNA molecule using the one or more calibrated base calling metrics. Provided herein are also methods for determining the likelihood that a subject has a disease or condition, such as cancer.

### CROSS-REFERENCE TO RELATED APPLICATIONS

The present application is a Continuation of International Patent Application No. PCT/US2024/061535, filed Dec. 20, 2024, which claims the benefit of priority of U.S. Provisional Patent Application No. 63/613,592, filed Dec. 21, 2023, which is incorporated by reference herein in its entirety for all purposes.

### SEQUENCE LISTING

The present application contains a sequence listing that has been submitted electronically in XML format. Said XML copy, created on Dec. 17, 2024, is named “01228-0041-00PCT.xml” and is 6,354 bytes in size. The information in the electronic format of the sequence listing is incorporated herein by reference in its entirety.

### FIELD OF THE INVENTION

The present disclosure provides methods and compositions related to base calling in a sequencing by synthesis reaction performed on a converted DNA molecule. Such methods are important for accurately detecting the methylation status and variants present in DNA, which, in turn, can be important for inferring information about the cells and subject from which the DNA sample is derived. In some embodiments, the DNA molecule is from a subject having or suspected of having a disease or disorder, such as cancer.

### INTRODUCTION

Base-resolution or single-site methylation (SSM) sequencing methods in which a base conversion procedure is used may suffer sequencing quality and yield issues on sequencing by synthesis (SBS)-based next generation sequencing (NGS) platforms, such as Illumina NGS sequencing platforms. The bases sequenced at the beginning of “read 1” may be used to calibrate base calling metrics, using an assumption that the sequenced libraries are sufficiently complex (also referred to as “diverse”), i.e., that all four bases (A, C, G, and T) will be present with some expected distribution (near random) at each cycle in each read. Well-balanced or high complexity libraries have roughly equal proportions of all four nucleotides in each cycle throughout the sequencing run. Low complexity libraries (e.g., that have undergone a base conversion procedure that deaminates unmethylated cytosines) have a high proportion of certain nucleotides and a low proportion of other nucleotides in a cycle. Finally, libraries that have adapters with in-line barcodes or other molecular identifiers and low complexity inserts (e.g., that have undergone a base conversion procedure that deaminates unmethylated cytosines), or that are prepared by ligating adapters with conversion-resistant nucleotides followed by undergoing a base conversion procedure, can have regions of low complexity and high complexity.

Sequence complexity is important for run performance and high-quality data generation, particularly in the early (e.g., first 25) cycles of a sequencing run because this is when various base calling metrics, e.g., the clusters passing filter, phasing/pre-phasing, and color matrix corrections, may be calibrated. Thus, because the assumption of complexity in the sequenced DNA is used to calibrate the base calling software for each sequencing run, sequencing DNA libraries with low or reduced complexity reduces the accuracy of software-based analyses, resulting in reduced sequencing yield and base call quality.

Some SSM methods (such as bisulfite sequencing, EM-seq, and DM-seq) deaminate or otherwise convert unmethylated cytosines to uracils, which are amplified and sequenced as thymines, while methylated cytosines (or mCpG only in DM-seq) are not deaminated and sequenced as cytosines. This results in DNA libraries with low complexity, that lead to significantly reduced NGS yields and/or quality when sequenced by themselves on Illumina instruments. For example, low complexity libraries can lead to a 50% (or more) yield reduction compared to sequencing yields of standard (non-converted) DNA libraries.

A common method used to reduce the sequencing yield and/or quality loss with SSM sequencing is to increase the base diversity of the total DNA being sequenced in a run by combining an SSM library (or libraries) with another high diversity library (or libraries), such as the PhiX library, in a pool to run on the same NGS flow cell. The PhiX library is derived from the small, well-characterized bacteriophage genome, PhiX. The PhiX library has an average size of 500 bp and a balanced base composition at approximately 45% GC and approximately 55% AT. See Illumina (2023). What is the PhiX Control v3 Library and what is its function in Illumina Next Generation Sequencing? available on knowledge.illumina.com at/library-preparation/general/library-preparation-general-reference_material-list/000001545. Depending on the NGS instrument and SSM library complexity, the required ‘spike in’ amount can vary from 5-50%. The base diversity of the pooled libraries (sample SSM library or libraries spiked with one or more high complexity libraries, such as PhiX) can be raised to an acceptable level to yield high quality sequencing in this way. However, while this results in increased quality and overall sequencing yield, the per (SSM) sample sequencing yield is reduced as compared to a standard (non-SSM) sequencing run. In other words, the total number of usable reads per flow cell for SSM libraries is reduced. In many applications, such as methylation sequencing in a clinical setting, this ‘spiking in’ of a high complexity library results in higher sequencing costs per sample as compared to standard non-SSM sequencing. In academic research settings it may be acceptable (albeit logistically challenging) to sequence SSM libraries in the same sequencing run with high complexity libraries from another project. For commercial and clinical sequencing pipelines, however, it is often not acceptable to sequence multiple projects or products in the same NGS run due to one or more of regulatory guidelines, turnaround time, and/or batch constraints.

Accordingly, improved methods for accurately detecting the modification status (e.g., methylation status) of nucleobases in DNA molecules sequenced using sequencing by synthesis-based sequencing platforms (such as Illumina NGS platforms) are needed. The present disclosure aims to meet this need, provide other benefits, or at least provide the public with a useful choice.

### SUMMARY

The present disclosure provides an improved SSM workflow for use in sequencing by synthesis-based sequencing (such as Illumina NGS). The workflow results in both improved base calling accuracy (e.g., of modified bases, such as methylated bases) and higher sequencing qualities and yields than theoretically achievable by current approaches (such as standard Illumina NGS). In some embodiments, the methods disclosed herein may reduce SSM workflow sequencing costs, such as by approximately 50%. The following exemplary embodiments are provided.

Embodiment 1 is a method comprising:
(a) performing a sequencing by synthesis reaction on a converted DNA molecule with a sequencing by synthesis instrument, the converted DNA molecule comprising:
ligated adapters;
a converted region comprising one or more nucleobases that have been converted by a conversion procedure; and a resistant region comprising one or more nucleobases that are resistant to the conversion procedure;
wherein the sequencing by synthesis reaction comprises extending a sequencing primer that binds to the converted DNA molecule upstream of the converted region and the resistant region;
(b) calibrating one or more base calling metrics of the sequencing by synthesis instrument based at least in part on data from the resistant region, thereby providing one or more calibrated base calling metrics; and
(c) calling at least a portion of nucleobases in the converted region using the one or more calibrated base calling metrics.

Embodiment 2 is a method comprising:
(a) ligating adapters to a DNA molecule, wherein the adapters comprise a resistant region comprising one or more nucleobases that are resistant to a conversion procedure and the DNA molecule comprises one or more nucleobases that are substrates for the conversion procedure, thereby producing an adapted DNA molecule;
(b) performing the conversion procedure on the adapted DNA molecule, thereby producing a converted DNA molecule comprising a converted region, the converted region comprising one or more nucleobases that have been converted by the conversion procedure;
(c) performing a sequencing by synthesis reaction on the converted DNA molecule with a sequencing by synthesis instrument;
wherein the sequencing by synthesis reaction comprises extending a sequencing primer that binds to the converted DNA molecule upstream of the converted region and the resistant region;
(d) calibrating one or more base calling metrics of the sequencing by synthesis instrument based at least in part on data from the resistant region, thereby providing one or more calibrated base calling metrics; and
(e) calling at least a portion of nucleobases in the converted region using the one or more calibrated base calling metrics.

Embodiment 3 is a method comprising:
(a) subjecting a DNA molecule comprising one or more nucleobases that are substrates for a conversion procedure to end repair, wherein the end repair comprises extending a recessed 3′ end of the DNA molecule using a DNA polymerase and deoxyribonucleotides comprising a nucleobase that is resistant to the conversion procedure, thereby generating an end-repaired DNA molecule comprising a resistant region that comprises the nucleobase resistant to the conversion procedure;
(b) ligating adapters to the end-repaired DNA molecule, thereby producing an adapted DNA molecule;
(c) performing the conversion procedure on the adapted DNA molecule, thereby producing a converted DNA molecule comprising a converted region, the converted region comprising one or more nucleobases that have been converted by the conversion procedure;
(d) performing a sequencing by synthesis reaction on the converted DNA molecule with a sequencing by synthesis instrument;
wherein the sequencing by synthesis reaction comprises extending a sequencing primer that binds to the converted DNA molecule upstream of the converted region and the resistant region;
(e) calibrating one or more base calling metrics of the sequencing by synthesis instrument based at least in part on data from the resistant region, thereby providing one or more calibrated base calling metrics; and
(f) calling at least a portion of nucleobases in the converted region using the one or more calibrated base calling metrics.

Embodiment 4 is the method of claim 1 or claim 3, wherein the ligating seals one or more nicks present in the end-repaired DNA.

Embodiment 5 is the method of the immediately preceding claim, wherein the end repair is performed with a DNA polymerase which does not have 5′-3′ exonuclease activity and/or is not a strand displacing DNA polymerase.

Embodiment 6 is the method of the immediately preceding claim, wherein the DNA polymerase is T4 DNA polymerase, T7 DNA polymerase, or Klenow fragment.

Embodiment 7 is the method of any one of the preceding claims, wherein the adapter is a Y-shaped adapter that comprises a first strand and a second strand.

Embodiment 8 is the method of the immediately preceding claim, wherein
(a) the first strand comprises a first arm region and a first stem region; and
(b) the second strand comprises a second arm region and a second stem region, wherein the second stem region is configured to anneal to the first stem region and the second arm region is configured not to anneal to the first arm region.

Embodiment 9 is the method of the immediately preceding claim, wherein the first arm region and second arm region each comprise at least one nucleobase that is resistant to the conversion procedure.

Embodiment 10 is the method of claim 8 or claim 9, wherein
(a)(i) the number of nucleobases with a base-pairing specificity complementary to the nucleobases that are resistant to the conversion procedure in the first arm region is greater than the number of nucleobases that are resistant to the conversion procedure in the first arm region, and/or (ii) the number of nucleobases that are resistant to the conversion procedure in the first arm region is less than 25% of the number of nucleobases in the first arm region; and
(b)(i) the number of nucleobases with a base-pairing specificity complementary to the nucleobases that are resistant to the conversion procedure in the second arm region is greater than the number of nucleobases that are resistant to the conversion procedure in the second arm region and/or (ii) the number of nucleobases that are resistant to the conversion procedure in the second arm region is less than 25% of the number of nucleobases in the second arm region.

Embodiment 11 is the method of any one of claims 8-10, wherein the first arm region is located 5′ of the first stem region and the second arm region is located 3′ of the second stem region.

Embodiment 12 is the method of any one of the preceding claims, wherein the nucleobase that is resistant to the conversion procedure comprises a modified nucleobase.

Embodiment 13 is the method of the immediately preceding claim, wherein the modified nucleobase comprises 4-methylcytosine (4mC), 5-methylcytosine (5mC), 5-hydroxymethylcytosine (5hmC), N6-methyladenosine (6 mA), bromodeoxyuridine (BrdU), 8-oxoguanine (8oxoG), 5-pyrrolo cytosine, 5-glucoylhydroxymethylated (5-ghmC), 5-caryboxylcytosine (5-caC), and/or 5-propynyl cytosine.

Embodiment 14 is the method of any one of the preceding claims, wherein the nucleobase that is resistant to the conversion procedure is a modified cytosine, optionally wherein the modified cytosine is 5-methylcytosine or 5-hydroxymethylcytosine.

Embodiment 15 is the method of any one of claims 8-14, wherein the first arm region, the second arm region, the first stem region, and/or the second stem region comprise one or more modified cytosines, optionally wherein the one or more modified cytosines are 5-methylcytosine, 5-hydroxymethylcytosine, 5-caryboxylcytosine (5-caC), and/or 5-propynyl cytosine.

Embodiment 16 is the method of any one of claims 8-15, wherein at least 50%, at least 60%, at least 70%, at least 75%, at least 80%, at least 85%, at least 90%, or at least 95% of the cytosines in the first arm region, the second arm region, the first stem region, and/or the second stem region are modified cytosines.

Embodiment 17 is the method of any one of claims 8-16, wherein the first arm region, the second arm region, the first stem region, and/or the second stem region are substantially free of unmodified cytosines.

Embodiment 18 is the method of any one of the preceding claims, wherein the resistant region is at least about 10, at least about 11, at least about 12, at least about 13, at least about 14, at least about 15, at least about 16, at least about 17, at least about 18, at least about 19, or at least about 20 nucleotides in length.

Embodiment 19 is the method of any one of the preceding claims, wherein the resistant region is about 10-40, about 10-35, about 10-30, about 10-25, about 10-20, about 10-15, about 15-40, about 15-35, about 15-30, about 15-25, about 15-20, about 20-40, about 20-35, about 20-30, or about 20-25 nucleotides in length.

Embodiment 20 is the method of any one of the preceding claims, wherein the resistant region is located 3′ of the converted region.

Embodiment 21 is the method of any one of the preceding claims, wherein the resistant region comprises at least 2, at least 3, at least 4, at least 5, at least 6, at least 7, at least 8, at least 9, at least 10, at least 11, at least 12, at least 13, at least 14, or at least 15 nucleobases that are resistant to the conversion procedure.

Embodiment 22 is the method of any one of the preceding claims, wherein the resistant region comprises 2-30, 2-25, 2-20, 2-15, 2-10, 2-9, 2-8, 2-7, 2-6, 2-5, 2-4, or 2-3 nucleobases that are resistant to the conversion procedure.

Embodiment 23 is the method of any one of the preceding claims, wherein the ligation is a sticky-end ligation.

Embodiment 24 is the method of any one of the preceding claims, wherein the one or more base calling metrics comprises clusters passing filter, phasing/pre-phasing, and/or color matrix corrections values.

Embodiment 25 is the method of any one of the preceding claims, wherein the sequencing by synthesis reaction comprises sequencing the DNA in a manner that distinguishes the first nucleobase from the second nucleobase.

Embodiment 26 is the method of any one of the preceding claims, wherein the sequencing by synthesis reaction comprises next generation sequencing.

Embodiment 27 is the method of any one of the preceding claims, wherein the sequencing by synthesis reaction comprises generating a plurality of sequencing reads and mapping the plurality of sequencing reads to one or more reference sequences to generate mapped sequence reads.

Embodiment 28 is the method of any one of the preceding claims, further comprising determining an epigenetic modification status of at least a portion of the nucleobases of the DNA.

Embodiment 29 is the method of any one of the preceding claims, further comprising performing an A-tailing reaction.

Embodiment 30 is the method of the immediately preceding claim, wherein the end-repair and the A-tailing reaction are performed in the same reaction mixture, optionally wherein the end-repair and the A-tailing reaction are performed a single tube and/or optionally wherein the end-repair and the A-tailing reaction are performed without an intervening clean-up step.

Embodiment 31 is the method of the immediately preceding claim, wherein the A-tailing is performed using a DNA polymerase that does not possess 5′-3′ exonuclease activity and/or is not a strand displacing DNA polymerase, optionally wherein the DNA polymerase is HemoKlen Taq.

Embodiment 32 is the method of claim 29 or claim 30, wherein the A-tailing is performed using a thermostable DNA polymerase.

Embodiment 33 is the method of claim 20 or claim 30, wherein the A-tailing is performed using Taq DNA polymerase, Tfl DNA Polymerase, Bst DNA Polymerase, Large Fragment or Tth DNA polymerase.

Embodiment 34 is the method of claim 29, wherein the end-repair and the A-tailing reaction are performed as separate reactions, wherein a reaction clean-up step is performed after the end-repair and before the A-tailing reaction.

Embodiment 35 is the method of claim 34, wherein the reaction clean-up step removes unincorporated dNTPs.

Embodiment 36 is the method of claim 34 or claim 35, wherein the A-tailing is performed using a DNA polymerase that does not possess 3′-5′ exonuclease activity, optionally wherein the DNA polymerase is Klenow Fragment lacking 3′-5′ exonuclease activity.

Embodiment 37 is the method of claim 30 or claim 34, wherein the A-tailing is performed using a DNA polymerase that possesses 5′-3′ exonuclease activity and/or is a strand displacing DNA polymerase.

Embodiment 38 is the method of any one of claims 29-37, wherein the A tailing reaction is performed at a higher temperature than the end repair, optionally wherein the end repair is performed at about 15-35 °C and/or the A tailing is performed at a temperature over about 60 °C, further optionally wherein the temperature over 60 °C is about 60 °C-75 °C.

Embodiment 39 is the method of any one of the preceding claims, wherein the conversion procedure comprises deamination of unmodified cytosines of the DNA to uracil.

Embodiment 40 is the method of any one of the preceding claims, wherein the conversion procedure comprises contacting the DNA or a subsample thereof with a cytosine deaminase.

Embodiment 41 is the method of the immediately preceding claim, wherein the cytosine deaminase is an APOBEC enzyme, optionally wherein the APOBEC enzyme is APOBEC3A.

Embodiment 42 is the method of any one of the preceding claims, wherein the conversion procedure comprises enzymatic protection of one or more modified nucleobases of the DNA.

Embodiment 43 is the method of the immediately preceding claim, wherein the enzymatic protection comprises glucosylation of the 5-hydroxymethylcytosines of the DNA, optionally wherein the glucosylation comprises contacting the DNA with beta-glucosyltransferase.

Embodiment 44 is the method of any one of the preceding claims, wherein the conversion procedure comprises contacting the DNA or a subsample thereof with a ten-eleven translocation (TET) enzyme.

Embodiment 45 is the method of any one of the preceding claims, wherein the conversion procedure comprises subjecting the DNA or a subsample thereof to a procedure that affects a first nucleobase of the DNA differently from a second nucleobase of the DNA, wherein the first nucleobase is a modified or unmodified nucleobase, the second nucleobase is a modified or unmodified nucleobase different from the first nucleobase, and the first nucleobase and the second nucleobase have the same base pairing specificity.

Embodiment 46 is the method of the immediately preceding claim, wherein the first nucleobase is an unmodified cytosine and the second nucleobase is a modified cytosine.

Embodiment 47 is the method of the immediately preceding claim, wherein the modified cytosine is 5-methylcytosine.

Embodiment 48 is the method of claim 37, wherein the modified cytosine is 5-hydroxymethylcytosine.

Embodiment 49 is the method of any one of claims 45-48, wherein the procedure that affects a first nucleobase of the DNA differently from a second nucleobase of the DNA chemically converts the first or second nucleobase such that the base pairing specificity of the converted nucleobase is altered.

Embodiment 50 is the method of any one of claims 45-49, wherein the procedure that affects a first nucleobase of the DNA differently from a second nucleobase of the DNA is methylation-sensitive conversion.

Embodiment 51 is the method of the immediately preceding claim, wherein the methylation-sensitive conversion is bisulfite conversion, oxidative bisulfite (Ox-BS) conversion, Tet-assisted bisulfite (TAB) conversion, APOBEC-coupled epigenetic (ACE) conversion, enzymatic methyl-seq (EM-seq), or single-enzyme 5-methylctyosine sequencing (SEM-seq) method.

Embodiment 52 is the method of the immediately preceding claim, wherein the Tet-assisted conversion further comprises a substituted borane reducing agent, optionally wherein the substituted borane reducing agent is 2-picoline borane, borane pyridine, tert-butylamine borane, or ammonia borane.

Embodiment 53 is the method of any one of the preceding claims, wherein the conversion procedure comprises contacting the DNA with a CpG-specific DNA methyltransferase (MTase) or a CpG-specific carboxymethyltransferase (CxMTase), a methyl donor or a carboxymethyl donor, and a cytosine deaminase.

Embodiment 54 is the method of the immediately preceding claim, wherein the cytosine deaminase is an APOBEC enzyme, optionally wherein the APOBEC enzyme is APOBEC3A.

Embodiment 55 is the method of any one of the preceding claims, wherein the adapters comprise at least one tag.

Embodiment 56 is the method of the immediately preceding claim, wherein the at least one tag comprises a molecular barcode.

Embodiment 57 is the method of any one of the preceding claims, wherein the DNA is cell-free DNA.

Embodiment 58 is the method of claim 57, wherein the cell-free DNA is in an amount between 1 ng and 500 ng.

Embodiment 59 is the method of any one of the preceding claims, wherein the DNA is from a blood sample and/or a tissue sample.

Embodiment 60 is the method of claim 59, wherein the blood sample is a whole blood sample, a plasma sample, a buffy coat sample, a leukapheresis sample, or a PBMC sample.

Embodiment 61 is the method of any one of the preceding claims, wherein the DNA and/or the sample is from a subject.

Embodiment 62 is the method of claim 61, wherein the subject is an animal.

Embodiment 63 is the method of claim 61 or claim 62, wherein the subject is a human.

Embodiment 64 is the method of any one of claims 59-63, wherein the blood sample is fractionated prior to enriching for at least one epigenetic target region sets of DNA.

Embodiment 65 is the method of any one of claims 61-64, wherein the subject has or is at risk of having a cancer.

Embodiment 66 is the method of any one of claims 61-65, further comprising determining the presence or status of a cancer in the subject.

Embodiment 67 is the method of any one of claims 61-66, further comprising determining the likelihood that the subject has an infection.

Embodiment 68 is the method of any one of claims 61-67, further comprising determining the likelihood that the subject has a transplant rejection.

Embodiment 69 is a Y-shaped oligonucleotide adapter comprising first and second strands, wherein:
(a) the first strand comprises a first arm region and a first stem region;
(b) the second strand comprises a second arm region and a second stem region, wherein the second stem region is configured to anneal to the first stem region and the second arm region is configured not to anneal to the first arm region;
(c) the first arm region and second arm region each comprise one or more modified nucleobases that are resistant to a conversion procedure; and
(d)(i) the number of nucleobases with a base-pairing specificity complementary to the modified nucleobases that are resistant to the conversion procedure in the first arm region is greater than the number of modified nucleobases that are resistant to the conversion procedure in the first arm region, and/or (ii) the number of modified nucleobases that are resistant to the conversion procedure in the first arm region is less than 25% of the number of nucleobases in the first arm region; and
(e)(i) the number of nucleobases with a base-pairing specificity complementary to the modified nucleobases that are resistant to the conversion procedure in the second arm region is greater than the number of modified nucleobases that are resistant to the conversion procedure in the second arm region and/or (ii) the number of modified nucleobases that are resistant to the conversion procedure in the second arm region is less than 25% of the number of nucleobases in the second arm region.

Embodiment 70 is the Y-shaped oligonucleotide adapter of the immediately preceding claim, wherein the modified nucleobase comprises 4-methylcytosine (4mC), 5-methylcytosine (5mC), 5-hydroxymethyl-cytosine (5hmC), N6-methyladenosine (6 mA), bromodeoxyuridine (BrdU), 8-oxoguanine (8oxoG), 5-pyrrolo cytosine, 5-glucoylhydroxymethylated (5-ghmC), 5-caryboxylcytosine (5-caC), and/or 5-propynyl cytosine.

Embodiment 71 is the Y-shaped oligonucleotide adapter of claim 69 or claim 70,
wherein the nucleobase that is resistant to the conversion procedure is a modified cytosine, optionally wherein the modified cytosine is 5-methylcytosine or 5-hydroxymethylcytosine.

Embodiment 72 is a Y-shaped oligonucleotide adapter comprising first and second strands, wherein:
(a) the first strand comprises a first arm region and a first stem region;
(b) the second strand comprises a second arm region and a second stem region, wherein the second stem region is configured to anneal to the first stem region and the second arm region is configured not to anneal to the first arm region;
(c) the first arm region and second arm region each comprise modified cytosines; and
(d)(i) the number of guanines in the first arm region is greater than the number of modified cytosines in the first arm region, and/or (ii) the number of modified cytosines in the first arm region is less than 25% of the number of nucleobases in the first arm region; and
(e)(i) the number of guanines in the second arm region is greater than the number of modified cytosines in the second arm region and/or (ii) the number of modified cytosines in the second arm region is less than 25% of the number of nucleobases in the second arm region.

Embodiment 73 is the Y-shaped oligonucleotide adapter of the immediately preceding claim, wherein the modified cytosine comprises 4-methylcytosine (4mC), 5-methylcytosine (5mC), 5-hydroxymethyl-cytosine (5hmC), 5-pyrrolo cytosine, 5-glucoylhydroxymethylated (5-ghmC), 5-caryboxylcytosine (5-caC), and/or 5-propynyl cytosine.

Embodiment 74 is the Y-shaped oligonucleotide adapter of any one of claims 71-73, wherein the modified cytosine is 5-methylcytosine.

Embodiment 75 is the Y-shaped oligonucleotide adapter of any one of claims 71-73, wherein the modified cytosine is 5-hydroxymethylcytosine.

Embodiment 76 is the Y-shaped oligonucleotide adapter of any one of claims 69-75, wherein the first arm region is located 5′ of the first stem region and the second arm region is located 3′ of the second stem region.

Embodiment 77 is the Y-shaped oligonucleotide adapter of any one of claims 69-76, wherein the first arm region, the second arm region, the first stem region, and/or the second stem region comprise one or more modified cytosines, optionally wherein the one or more modified cytosines are 5-methylcytosine, 5-hydroxymethylcytosine, 5-caryboxylcytosine (5-caC), and/or 5-propynyl cytosine.

Embodiment 78 is the Y-shaped oligonucleotide adapter of any one of claims 69-77, wherein at least 50%, at least 60%, at least 70%, at least 75%, at least 80%, at least 85%, at least 90%, or at least 95% of the cytosines in the first arm region, the second arm region, the first stem region, and/or the second stem region are modified cytosines.

Embodiment 79 is the Y-shaped oligonucleotide adapter of any one of claims 69-78, wherein the first arm region, the second arm region, the first stem region, and/or the second stem region are substantially free of unmodified cytosines.

Embodiment 80 is the Y-shaped oligonucleotide adapter of any one of the preceding claims, further comprising at least one tag.

Embodiment 81 is the Y-shaped oligonucleotide adapter of the immediately preceding claim, wherein the at least one tag comprises a molecular barcode.

In some embodiments, the results of the methods disclosed herein are used as an input to generate a report. The report may be in a paper or electronic format. For example, true copy number variation, as obtained by the methods disclosed herein, or information derived therefrom, can be displayed directly in such a report. Alternatively or additionally, diagnostic information or therapeutic recommendations which are at least in part based on the methods disclosed herein can be included in the report.

The various steps of the methods disclosed herein may be carried out at the same or different times, in the same or different geographical locations, e.g. countries, and/or by the same or different people.

Additional advantages will be set forth in part in the description which follows or may be learned by practice. The advantages will be realized and attained by means of the elements and combinations particularly pointed out in the appended claims.

### BRIEF DESCRIPTION OF THE DRAWINGS

FIG. 1A illustrates an exemplary standard double-stranded DNA library preparation workflow for methylation sequencing, including standard end-repair and A-tailing and epigenetic base conversion steps.

FIG. 1B illustrates an exemplary double-stranded DNA library preparation workflow for methylation sequencing according to certain embodiments disclosed herein. DNA useful in the disclosed embodiments can include cell-free DNA and/or DNA collected from a sample comprising cells (such as a blood sample (e.g., a whole blood sample, a buffy coat sample, a leukapheresis sample, or a PBMC sample)).

FIG. 2 is a schematic diagram of an example of a system suitable for use with some embodiments of the disclosure.

FIG. 3 illustrates an exemplary standard Y-shaped adapter (left) and an exemplary “complement” Y-shaped adapter suitable for use with some embodiments of the disclosure (right). The standard Y-shaped adapter comprises a 5′ read1 adapter strand, a 3′ read2 adapter strand, and an exemplary molecular barcode. The “complement” Y-shaped adapter comprises 5′ read2 adapter strand, 3′ read1 adapter strand, and an exemplary molecular barcode. The complement Y-shaped adapter (right) comprises the reverse complement of the Illumina-specific sequences of a current/standard NGS adapter (left). The sequences of each strand of the exemplary Y-shaped adapters (SEQ ID NOs: 1-4) are as follows. SEQ ID NO: 1 (GATCTACACTCTTTCCCTACACGACGCTCTTCCGATCTCGATGTGT) is a 5′ read1 adapter strand of an exemplary standard Y-shaped adapter. SEQ ID NO: 2 (CACTGACCTCAAGTCTGCACACGAGAAGGCTAGAGCTACAC) is a 3′ read2 adapter strand of an exemplary standard Y-shaped adapter. SEQ ID NO: 3 (GTGACTGGAGTTCAGACGTGTGCTCTTCCGATCTCGATGTGT) is a 5′ read2 adapter strand of an exemplary Y-shaped adapter as disclosed herein. SEQ ID NO: 4 (CTAGATGTGAGAAAGGGATGTGCTGCGAGAAGGCTAGAGCTACAC) is a 3′ read1 adapter strand of an exemplary Y-shaped adapter as disclosed herein.

### DETAILED DESCRIPTION OF CERTAIN EMBODIMENTS

Reference will now be made in detail to certain embodiments of the invention. While the invention will be described in conjunction with such embodiments, it will be understood that they are not intended to limit the invention to those embodiments. On the contrary, the invention is intended to cover all alternatives, modifications, and equivalents, which may be included within the invention as defined by the appended claims.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a nucleic acid” includes a plurality of nucleic acids, reference to “a cell” includes a plurality of cells, and the like.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the above specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims).

The section headings used herein are for organizational purposes and are not to be construed as limiting the disclosed subject matter in any way. In the event that any document or other material incorporated by reference contradicts any explicit content of this specification, including definitions, this specification controls.

### I. Definitions

As used herein, “cell-free DNA,” “cfDNA molecules,” or simply “cfDNA” includes DNA molecules that naturally occur in a subject in extracellular form (e.g., in blood, serum, plasma, or other bodily fluids such as lymph, cerebrospinal fluid, urine, or sputum). While the cfDNA originally existed in a cell or cells in a large complex biological organism, e.g., a mammal, it has undergone release from the cell(s) into a fluid found in the organism, and may be obtained from a sample of the fluid without the need to perform an in vitro cell lysis step.

As used herein, “buffy coat” refers to the portion of a blood (such as whole blood) or bone marrow sample that contains all or most of the white blood cells and platelets of the sample. The buffy coat fraction of a sample can be prepared from the sample using centrifugation, which separates sample components by density. For example, following centrifugation of a whole blood sample, the buffy coat fraction is situated between the plasma and erythrocyte (red blood cell) layers. The buffy coat can contain both mononuclear (e.g., T cells, B cells, NK cells, dendritic cells, and monocytes) and polymorphonuclear (e.g., granulocytes such as neutrophils and eosinophils) white blood cells.

As used herein, “leukapheresis” refers to a procedure in which white blood cells (leukocytes) are isolated from a sample of blood collected from a subject. Leukapheresis may be performed, e.g., obtain cells for research, diagnostic, prognostic, or monitoring purposes, such as those described herein. Thus, as used herein, a “leukapheresis sample” refers to a sample comprising leukocytes collected from a subject using leukapheresis.

As used herein, “peripheral blood mononuclear cells” or “PBMCs” refers to immune cells having a single, round nucleus that originate in bone marrow and are found in the peripheral circulation. Such cells include, e.g., lymphocytes (T cells, B cells, and NK cells) as well as monocytes, and are isolated from blood samples (such as from a whole blood sample collected from a subject) using density gradient centrifugation.

As used herein, “partitioning” of nucleic acids, such as DNA molecules, means separating, fractionating, sorting, or enriching a sample or population of nucleic acids into a plurality of subsamples or subpopulations of nucleic acids based on one or more modifications or features that is in different proportions in each of the plurality of subsamples or subpopulations.

Partitioning may include physically partitioning nucleic acid molecules based on the presence or absence of one or more methylated nucleobases. A sample or population may be partitioned into one or more partitioned subsamples or subpopulations based on a characteristic that is indicative of a genetic or epigenetic change or a disease state.

As used herein, “fragment” or “fragmenting” refers to the breaking or separation of a biological component, such as a nucleic acid molecule (such as DNA or RNA) into two or more pieces. Fragmentation, such as DNA fragmentation, can occur spontaneously or can be induced intentionally, such as using standard laboratory procedures, such as described herein. DNA fragmentation can be performed, for example, to prepare DNA (such as genomic DNA and/or DNA isolated from a sample comprising cells) for sequencing.

A “reaction cleanup” refers to the removal of contaminants such as salts, enzymes, unincorporated dNTPs, primers, ethidium bromide, and other impurities that can interfere with downstream analysis. For example, when a reaction cleanup is performed between end repair and an A-tailing reaction, it removes unincorporated dNTPs such that the A-tailing reaction can be performed solely in the presence of dATP (i.e., not dCTP, dGTP and dCTP, as used in the end tailing reaction). Reaction cleanups can be performed using commercially available kits such as MinElute Reaction Cleanup Kit (Qiagen)

“Synthesized regions,” also referred to as “regions of the end-repaired DNA that were synthesized during the end repair” refer to regions of the DNA that were not present in the DNA prior to the end repair and A-tailing reactions. They are regions which have been synthesized by the polymerases used in the end repair and/or A tailing reactions, if present. In instances where the A-tailing is performed in the same tube as the end repair reaction, all four types of dNTPs will be present, and thus the polymerases used for A-tailing may generate synthesized regions, e.g., through nick translation. In instances where the A-tailing is performed separately to the end repair reaction, and these steps are separated by a reaction cleanup, only dATP will be present in the A-tailing reaction, and thus the polymerases used for A-tailing will not typically generate synthesized regions because the dNTP components are not all present in the A-tailing reaction mix.

As used herein, “isolated” refers to a biological component (such as a nucleic acid molecule, protein, or cell) that has been substantially separated, produced apart from, or purified away from other components (for example, other components in a sample, cell, or organism in which the component naturally occurs). Nucleic acid molecules, proteins, or cells that have been “isolated” include those purified using standard purification methods. The term “isolated” or “purified” does not require absolute purity; rather, it is intended as a relative term. Thus, for example, an isolated biological component is one in which the biological component is more enriched in a preparation than the biological component is in its natural environment within a cell, organism, sample, or production vessel (for example, a cell culture system). For example, an isolated biological component can represent at least 50%, such as at least 70%, at least 80%, at least 90%, at least 95%, or greater, of the total biological component content of the preparation.

As used herein, “base pairing specificity” refers to the standard DNA base (A, C, G, or T) for which a given base most preferentially pairs. For example, unmodified cytosine and 5-methylcytosine have the same base pairing specificity (i.e., specificity for G) whereas uracil and cytosine have different base pairing specificity because uracil has base pairing specificity for A while cytosine has base pairing specificity for G. The ability of uracil to form a wobble pair with G is irrelevant because uracil nonetheless most preferentially pairs with A among the four standard DNA bases.

As used herein, “without substantially altering base pairing specificity” of a given nucleobase means that a majority of molecules comprising that nucleobase that can be sequenced do not have alterations of the base pairing specificity of the given nucleobase relative to its base pairing specificity as it was in the originally isolated sample. In some embodiments, 75%, 90%, 95%, or 99% of molecules comprising that nucleobase that can be sequenced do not have alterations of the base pairing specificity relative to its base pairing specificity as it was in the originally isolated sample. As used herein, “altered base pairing specificity” of a given nucleobase means that a majority of molecules comprising that nucleobase that can be sequenced have a base pairing specificity at that nucleobase relative to its base pairing specificity in the originally isolated sample.

As used herein, a modification or other feature is present in “a greater proportion” in a first sample or population of nucleic acid than in a second sample or population when the fraction of nucleotides with the modification or other feature is higher in the first sample or population than in the second population. For example, if in a first sample, one tenth of the nucleotides are mC, and in a second sample, one twentieth of the nucleotides are mC, then the first sample comprises the cytosine modification of 5-methylation in a greater proportion than the second sample.

As used herein, a “differentially methylated region” refers to a region of DNA having a detectably different degree of methylation in at least one cell or tissue type relative to the degree of methylation in the same region of DNA from at least one other cell or tissue type; or having a detectably different degree of methylation in at least one cell or tissue type obtained from a subject having a disease or disorder relative to the degree of methylation in the same region of DNA in the same cell or tissue type obtained from a healthy subject. In some embodiments, a differentially methylated region has a detectably higher degree of methylation (e.g., a hypermethylated region) in at least one cell or tissue type, such as at least one cancer cell type and/or at least one immune cell type, relative to the degree of methylation in the same region of DNA from at least one other cell or tissue type, such as other immune cell types, or from the same cell or tissue type from a healthy subject. In some embodiments, a differentially methylated region has a detectably lower degree of methylation (e.g., a hypomethylated region) in at least one cell or tissue type, such as at least one cancer cell type and/or at least one immune cell type, relative to the degree of methylation in the same region of DNA from at least one other cell or tissue type, such as other immune cell types, or from the same cell or tissue type from a healthy subject.

A nucleic acid is “produced by a tumor” if it originated from a tumor cell. Tumor cells are neoplastic cells that originated from a tumor, regardless of whether they remain in the tumor or become separated from the tumor (as in the cases, e.g., of metastatic cancer cells and circulating tumor cells). As used herein, “precancer” or a “precancerous condition” is an abnormality that has the potential to become cancer, wherein the potential to become cancer is greater than the potential if the abnormality was not present, i.e., was normal. Examples of precancer include but are not limited to adenomas, hyperplasias, metaplasias, dysplasias, benign neoplasias (benign tumors), premalignant carcinoma in situ, and polyps. It should be noted that certain types of carcinoma in situ are recognized in the field as cancerous, e.g., Stage 0 cancer, as opposed to premalignant.

The term “methylation” or “DNA methylation” refers to addition of a methyl group to a nucleotide base in a nucleic acid molecule. In some embodiments, methylation refers to addition of a methyl group to a cytosine at a CpG site (cytosine-phosphate-guanine site (i.e., a cytosine followed by a guanine in a 5′→3′ direction of the nucleic acid sequence)). In some embodiments, DNA methylation refers to addition of a methyl group to adenine, such as in N⁶-methyladenine (6 mA). In some embodiments, DNA methylation is 5-methylation (modification of the carbon in the 5th position of the cytosine ring). In some embodiments, 5-methylation refers to addition of a methyl group to the 5C position of the cytosine to create 5-methylcytosine (5mC). In some embodiments, methylation comprises a derivative of 5mC. Derivatives of 5mC include, but are not limited to, 5-hydroxymethylcytosine (5-hmC), 5-formylcytosine (5-fC), and 5-caryboxylcytosine (5-caC). In some embodiments, DNA methylation is 3C methylation (modification of the carbon in the 3^{rd} position of the cytosine ring). In some embodiments, 3C methylation comprises addition of a methyl group to the 3C position of the cytosine to generate 3-methylcytosine (3mC). Methylation can also occur at non-CpG sites, for example, methylation can occur at a CpA, CpT, or CpC site. DNA methylation can change the activity of methylated DNA region. For example, when DNA in a promoter region is methylated, transcription of the gene may be repressed. DNA methylation is critical for normal development and abnormality in methylation may disrupt epigenetic regulation. The disruption, e.g., repression, in epigenetic regulation may cause diseases, such as cancer. Promoter methylation in DNA may be indicative of cancer.

As used herein, “modified cytosine” refers to a cytosine in which at least one position of the cytosine has been substituted with a chemical moiety, such as a methyl or hydroxymethyl, that is different from the substituent at that position in unmodified cytosine. For the avoidance of doubt, “modified cytosine” does not include unmodified cytosine.

The term “hypermethylation” refers to an increased level or degree of methylation of nucleic acid molecule(s) relative to the other nucleic acid molecules within a population (e.g., sample) of nucleic acid molecules. In some embodiments, hypermethylated DNA can include DNA molecules comprising at least 1 methylated residue, at least 2 methylated residues, at least 3 methylated residues, at least 5 methylated residues, or at least 10 methylated residues.

The term “hypomethylation” refers to a decreased level or degree of methylation of nucleic acid molecule(s) relative to the other nucleic acid molecules within a population (e.g., sample) of nucleic acid molecules. In some embodiments, hypomethylated DNA includes unmethylated DNA molecules. In some embodiments, hypomethylated DNA can include DNA molecules comprising 0 methylated residues, at most 1 methylated residue, at most 2 methylated residues, at most 3 methylated residues, at most 4 methylated residues, or at most 5 methylated residues.

As used herein, “methylation status” can refer to the presence or absence of methyl group on a DNA base (e.g., cytosine) at a particular genomic position in a nucleic acid molecule. It can also refer to the degree of methylation in a nucleic acid sequence (e.g., highly methylated, low methylated, intermediately methylated or unmethylated nucleic acid molecules). The methylation status can also refer to the number of nucleotides methylated in a particular nucleic acid molecule.

As used herein, a “sequencing by synthesis reaction” refers to sequencing reactions (which are generally next-generation sequencing (NGS) reactions) that can determine the sequence of a DNA molecule by detecting the incorporation of each nucleotide into a complementary strand synthesized by a DNA polymerase. As the polymerase synthesizes a copy of a single strand of DNA, the incorporation of each nucleotide is monitored, such as by detection of fluorescently labeled nucleotides.

As used herein, “next-generation sequencing” or “NGS” refers to sequencing technologies having increased throughput as compared to traditional Sanger- and capillary electrophoresis-based approaches, for example, with the ability to generate hundreds of thousands of sequence reads at a time. Some examples of next-generation sequencing techniques include, but are not limited to, sequencing by synthesis, sequencing by ligation, and sequencing by hybridization. In some embodiments, next-generation sequencing includes the use of instruments capable of sequencing single molecules. Examples of commercially available instruments for performing next-generation sequencing include, but are not limited to, NextSeq, HiSeq, NovaSeq, MiSeq, Ion PGM and Ion GeneStudio S5.

As used herein, “clusters passing filter” refers to a base calling metric that provides an indication of signal purity from each cluster in a sequencing run (e.g., an NGS run). Clusters passing filters are commonly used in processing NGS data, e.g., in Illumina workflows. A cluster is a clonal grouping of template DNA, e.g., bound to the surface of a flow cell. Each cluster is typically seeded by a single, template DNA strand and is clonally amplified until the cluster has approximately 1000 copies. Each cluster (e.g., on the flow cell) produces a single sequencing read. During initial sequencing cycles (e.g., 1-25) of read 1, the least reliable clusters are removed, e.g., filtered (such as by a chastity filter, e.g., of Illumina NGS software), from the image extraction results. For example, Illumina sequencers may perform an internal quality filtering procedure called chastity filter. Thus, in an exemplary Illumina NGS analysis, clusters may “pass filter” if no more than 1 base call has a chastity value below 0.6 in the first 25 cycles.

Chastity is defined as the ratio of the brightest base intensity divided by the sum of the brightest and the second brightest base intensities. The “clusters passing filter” metric can then be used (such as in combination with other metrics, such as phasing/prephasing rates and/or color matrix correction values) in base calling and quality score calculations for all cycles in the run. See Illumina (2016), Optimizing Cluster Density on Illumina Sequencing Systems, available at illumina.com/content/dam/illumina-marketing/documents/products/other/miseq-overclustering-primer-770-2014-038.pdf.

As used herein, “phasing/prephasing” refers to a base calling metric that indicates the fraction of molecules that become phased or prephased per sequencing cycle. Phasing and pre-phasing are commonly used in processing NGS data, e.g., in Illumina workflows. Phasing and prephasing indicate the rate at which singular molecules in a cluster fall behind (“phasing”) or move ahead (“prephasing”) of the current cycle during the sequencing stage, e.g., of an NGS run (such as an Illumina NGS run). In other words, the phasing and prephasing rates define the fraction of molecules that become phased or prephased per cycle. During sequencing by synthesis, each DNA strand in a cluster extends by one base per cycle. A small proportion of strands may become out of phase with the current cycle, either falling a base behind (phasing) or jumping a base ahead (prephasing). The phasing and prephasing rates define the fraction of molecules that become phased or prephased per cycle. Calculation of these rates generally requires a balanced and random base composition in sequencing cycles 2-12. The “phasing/prephasing” metric is then used (typically in combination with other metrics, such as clusters passing filter and/or color matrix correction values) in base calling and quality score calculations for all cycles in the run. See Illumina (2013), Using a PhiX Controlfor HiSeq® Sequencing Runs, available at illumina.com/content/dam/illumina-support/documents/products/technotes/technote_phixcontrolv3.pdf.

As used herein, “color matrix correction values” refers to a base calling metric that is used to correct for cross talk between imaging channels in a sequencing analysis. Color matrix correction values are commonly used in processing NGS data, e.g., in Illumina workflows. Color matrix correction refers to a template created in the first few sequencing cycles (e.g., of an NGS analysis) that includes intensities from each imaging channel, and which is then used in all subsequent reads as well as for phasing/pre-phasing rates. Cross talk occurs when, for example, a cluster shows intensity in the cytosine channel and some intensity also shows in the adenine channel. Matrix-corrected intensities are generated with reduced or no cross talk, and differences in overall intensities between color channels are balanced. The “color matrix correction values” metric is then used (typically in combination with other metrics, such as phasing/prephasing rates and/or clusters passing filter) in base calling and quality score calculations for all cycles in the run. See Illumina (2003), What is nucleotide diversity and why is it important?, available at knowledge.illumina.com/instrumentation/general/instrumentation-general-reference_material-list/000001543.

As used herein, the form of the “originally isolated” sample refers to the composition or chemical structure of a sample at the time it was isolated and before undergoing any procedure that changes the chemical structure of the isolated sample. Similarly, a feature that is “originally present” in DNA molecules refers to a feature present in “original DNA molecules” or in DNA molecules “originally comprising” the feature before the DNA molecules undergo a procedure that changes the chemical structure of DNA molecules.

As used herein, “nucleic acid tag” refers to a short nucleic acid (e.g., less than about 500 nucleotides, about 100 nucleotides, about 50 nucleotides, or about 10 nucleotides in length), used to distinguish nucleic acids from different samples (e.g., representing a sample index), distinguish nucleic acids from different partitions (e.g., representing a partition tag) or different nucleic acid molecules in the same sample (e.g., representing a molecular barcode), of different types, or which have undergone different processing. The nucleic acid tag comprises a predetermined, fixed, non-random, random or semi-random oligonucleotide sequence. Such nucleic acid tags may be used to label different nucleic acid molecules or different nucleic acid samples or sub-samples. Nucleic acid tags can be single-stranded, double-stranded, or at least partially double-stranded. Nucleic acid tags optionally have the same length or varied lengths. Nucleic acid tags can also include double-stranded molecules having one or more blunt-ends, include 5′ or 3′ single-stranded regions (e.g., an overhang), and/or include one or more other single-stranded regions at other locations within a given molecule. Nucleic acid tags can be attached to one end or to both ends of the other nucleic acids (e.g., sample nucleic acids to be amplified and/or sequenced). Nucleic acid tags can be decoded to reveal information such as the sample of origin, form, or processing of a given nucleic acid. For example, nucleic acid tags can also be used to enable pooling and/or parallel processing of multiple samples comprising nucleic acids bearing different molecular barcodes and/or sample indexes in which the nucleic acids are subsequently being deconvolved by detecting (e.g., reading) the nucleic acid tags. Nucleic acid tags can also be referred to as identifiers (e.g., molecular identifier, sample identifier). Additionally, or alternatively, nucleic acid tags can be used as molecular identifiers (e.g., to distinguish between different molecules or amplicons of different parent molecules in the same sample or sub-sample). This includes, for example, uniquely tagging different nucleic acid molecules in a given sample, or non-uniquely tagging such molecules. In the case of non-unique tagging applications, a limited number of tags (i.e., molecular barcodes) may be used to tag each nucleic acid molecule such that different molecules can be distinguished based on their endogenous sequence information (for example, start and/or stop positions where they map to a selected reference genome, a sub-sequence of one or both ends of a sequence, and/or length of a sequence) in combination with at least one molecular barcode. Typically, a sufficient number of different molecular barcodes are used such that there is a low probability (e.g., less than about a 10%, less than about a 5%, less than about a 1%, or less than about a 0.1% chance) that any two molecules may have the same endogenous sequence information (e.g., start and/or stop positions, subsequences of one or both ends of a sequence, and/or lengths) and also have the same molecular barcode. Terms such as “library adaptors having distinct molecular barcodes” encompass library adaptors for uniquely or non-uniquely tagging molecules, in that regardless of whether the adaptors are for unique or non-unique tagging, distinct barcodes will be present in the population of adaptors.

As used herein, “subject” refers to an animal, such as a mammalian species (e.g., human) or avian (e.g., bird) species, or other organism, such as a plant. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or a predisposition to the disease, or an individual in need of therapy or suspected of needing therapy. The terms “individual” or “patient” are intended to be interchangeable with “subject”. For example, a subject can be an individual who has been diagnosed with having a cancer, is going to receive a cancer therapy, and/or has received at least one cancer therapy. The subject can be in remission of a cancer. As another example, the subject can be an individual who is diagnosed of having an autoimmune disease. As another example, the subject can be a female individual who is pregnant or who is planning on getting pregnant, who may have been diagnosed of or suspected of having a disease, e.g., a cancer, an auto-immune disease.

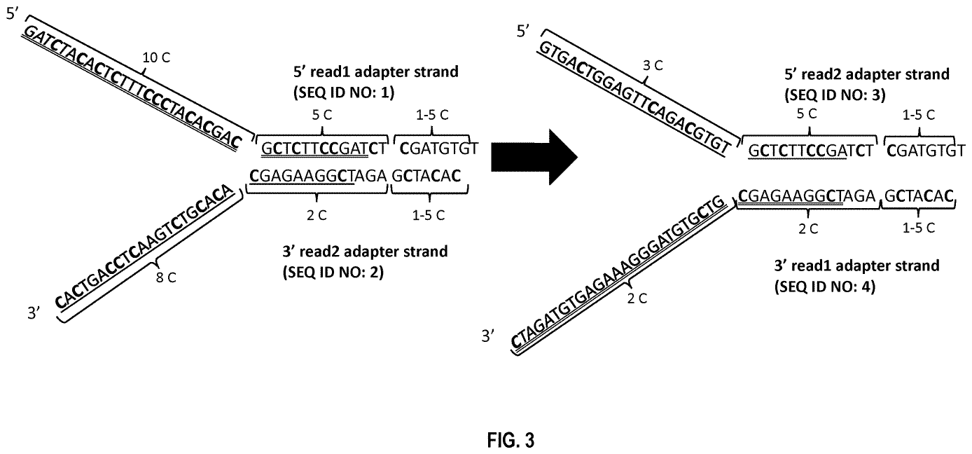

As used herein, a “Y-shaped adapter” refers to an adapter comprising two DNA strands comprising complementary and non-complementary parts, wherein the non-complementary parts form single-stranded arms. The adapter can be attached to a sample or insert DNA molecule, e.g., by ligation, such that the complementary (double-stranded) part of the adapter is proximal to the sample or insert DNA molecule. Prior to attachment, the double stranded portion of the Y-shaped adapter may have a blunt end or an overhang, e.g., of one to three nucleotides. The single stranded arms may or may not be of identical length. Unless context dictates otherwise, “Y-shaped adapter” as used herein refers to a Y-shaped adapter for use in the disclosed methods (such as the exemplary Y-shaped adapter shown in FIG. 3, left side).

The terms “or a combination thereof” and “or combinations thereof” as used herein refers to any and all permutations and combinations of the listed terms preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context. The “capture yield” of a collection of probes for a given target set refers to the amount (e.g., amount relative to another target set or an absolute amount) of nucleic acid corresponding to the target set that the collection of probes captures under typical conditions. Exemplary typical capture conditions are an incubation of the sample nucleic acid and probes at 65 °C for 10-18 hours in a small reaction volume (about 20 μL) containing stringent hybridization buffer. The capture yield may be expressed in absolute terms or, for a plurality of collections of probes, relative terms. When capture yields for a plurality of sets of target regions are compared, they are normalized for the footprint size of the target region set (e.g., on a per-kilobase basis). Thus, for example, if the footprint sizes of first and second target regions are 50 kb and 500 kb, respectively (giving a normalization factor of 0.1), then the DNA corresponding to the first target region set is captured with a higher yield than DNA corresponding to the second target region set when the mass per volume concentration of the captured DNA corresponding to the first target region set is more than 0.1 times the mass per volume concentration of the captured DNA corresponding to the second target region set. As a further example, using the same footprint sizes, if the captured DNA corresponding to the first target region set has a mass per volume concentration of 0.2 times the mass per volume concentration of the captured DNA corresponding to the second target region set, then the DNA corresponding to the first target region set was captured with a two-fold greater capture yield than the DNA corresponding to the second target region set.

“Capturing” one or more target nucleic acids or one or more nucleic acids comprising at least one target region refers to preferentially isolating or separating the one or more target nucleic acids or one or more nucleic acids comprising at least one target region from non-target nucleic acids or from nucleic acids that do not comprise at least one target region.

A “captured set” of nucleic acids or “captured” nucleic acids refers to nucleic acids that have undergone capture.

As used herein, a “capture moiety” is a molecule that allows affinity separation of molecules, such as nucleic acids, linked to the capture moiety from molecules lacking the capture moiety. Exemplary capture moieties include biotin, which allows affinity separation by binding to streptavidin linked or linkable to a solid phase or an oligonucleotide, which allows affinity separation through binding to a complementary oligonucleotide linked or linkable to a solid phase.

A “target region” refers to a genomic locus targeted for identification and/or capture, for example, by using probes (e.g., through sequence complementarity). A “target region set” or “set of target regions” refers to a plurality of genomic loci targeted for identification and/or capture, for example, by using a set of probes (e.g., through sequence complementarity).

“Specifically binds” in the context of a primer, a probe, or other oligonucleotide and a target sequence means that under appropriate hybridization conditions, the primer, oligonucleotide, or probe hybridizes to its target sequence, or replicates thereof, to form a stable hybrid, while at the same time formation of stable non-target hybrids is minimized. Thus, a primer or probe hybridizes to a target sequence or replicate thereof to a sufficiently greater extent than to a non-target sequence, to ultimately enable capture or detection of the target sequence.

Appropriate hybridization conditions are well-known in the art, may be predicted based on sequence composition, or can be determined by using routine testing methods (see, e.g., Sambrook et al., Molecular Cloning, A Laboratory Manual, 2^{nd} ed. (Cold Spring Harbor Laboratory Press, Cold Spring Harbor, NY, 1989) at §§ 1.90-1.91, 7.37-7.57, 9.47-9.51 and 11.47-11.57, particularly §§ 9.50-9.51, 11.12-11.13, 11.45-11.47 and 11.55-11.57, incorporated by reference herein).

“Sequence-variable target regions” refer to target regions that may exhibit changes in sequence such as nucleotide substitutions (i.e., single nucleotide variations), insertions, deletions, or gene fusions or transpositions in neoplastic cells (e.g., tumor cells and cancer cells) relative to normal cells. A sequence-variable target region set is a set of sequence-variable target regions. In some embodiments, the sequence-variable target regions are target regions that may exhibit changes that affect less than or equal to 50 contiguous nucleotides, e.g., less than or equal to 40, 30, 20, 10, 5, 4, 3, 2, or 1 nucleotides.

“Epigenetic target regions” refers to target regions that may show sequence-independent differences in different cell or tissue types (e.g., different types of immune cells) or in neoplastic cells (e.g., tumor cells and cancer cells) relative to normal cells; or that may show sequence-independent differences (i.e., in which there is no change to the nucleotide sequence, e.g., differences in methylation, nucleosome distribution, or other epigenetic features) in DNA, e.g., from different cell types or from subjects having cancer relative to DNA from healthy subjects. Examples of sequence-independent changes include, but are not limited to, changes in methylation (increases or decreases), nucleosome distribution, fragmentation patterns, CCCTC-binding factor (“CTCF”) binding, transcription start sites (e.g., with respect to any one of more of binding of RNA polymerase components, binding of regulatory proteins, fragmentation characteristics, and nucleosomal distribution), and regulatory protein binding regions. Epigenetic target region sets thus include, but are not limited to, hypermethylation variable target region sets, hypomethylation variable target region sets, and fragmentation variable target region sets, such as CTCF binding sites and transcription start sites. For present purposes, loci susceptible to neoplasia-, tumor-, or cancer-associated focal amplifications and/or gene fusions may also be included in an epigenetic target region set because detection of a change in copy number by sequencing or a fused sequence that maps to more than one locus in a reference genome tends to be more similar to detection of exemplary epigenetic changes discussed above than detection of nucleotide substitutions, insertions, or deletions, e.g., in that the focal amplifications and/or gene fusions can be detected at a relatively shallow depth of sequencing because their detection does not depend on the accuracy of base calls at one or a few individual positions. An epigenetic target region set is a set of epigenetic target regions.

The terms “agent that recognizes a modified nucleobase in DNA,” such as an “agent that recognizes a modified cytosine in DNA” refers to a molecule or reagent that binds to or detects one or more modified nucleobases in DNA, such as methyl cytosine. A “modified nucleobase” is a nucleobase that comprises a difference in chemical structure from an unmodified nucleobase. In the case of DNA, an unmodified nucleobase is adenine, cytosine, guanine, or thymine. In some embodiments, a modified nucleobase is a modified cytosine. In some embodiments, a modified nucleobase is a methylated nucleobase. In some embodiments, a modified cytosine is a methyl cytosine, e.g., a 5-methyl cytosine. In such embodiments, the cytosine modification is a methyl. Agents that recognize a methyl cytosine in DNA include but are not limited to “methyl binding reagents,” which refer herein to reagents that bind to a methyl cytosine. Methyl binding reagents include but are not limited to methyl binding domains (MBDs) and methyl binding proteins (MBPs) and antibodies specific for methyl cytosine. In some embodiments, such antibodies bind to 5-methyl cytosine in DNA. In some such embodiments, the DNA may be single-stranded or double-stranded. Suitable agents include agents that recognize modified nucleotides in double-stranded DNA, single-stranded DNA, and both double-stranded and single-stranded DNA.

“Substantially free” means free to a sufficient extent that the relevant properties are not meaningfully impacted by the presence of a minor impurity, such as the presence of a minor number of modified cytosines in a first arm region, a second arm region, a first stem region, and/or a second stem region of a Y-shaped adapter as disclosed herein. Substantially free does not require 100% (such as exactly 100% of the cytosines of a first arm region, a second arm region, a first stem region, and/or a second stem region of a Y-shaped adapter are unmodified cytosines) but can include an amount equal to or greater than 90%, such as 91%, 92%, 93%, 94%, 95%, 96%, 97%, 98%, 99%, or 100% (such as 90%-100% of the cytosines of a first arm region, a second arm region, a first stem region, and/or a second stem region of a Y-shaped adapter are modified cytosines). In some embodiments, a first arm region, a second arm region, a first stem region, and/or a second stem region of a Y-shaped adapter as disclosed herein are “substantially free” of unmodified cytosines if at least 90% of the cytosines are modified cytosines.

“Or” is used in the inclusive sense, i.e., equivalent to “and/or,” unless the context requires otherwise.

### II. Exemplary Methods

### A. Overview

The present disclosure provides methods of performing a sequencing by synthesis reaction comprising a step of modified end repair (such as end repair performed using a modified dCTP, such as d5mCTP) and/or the use of modified NGS adapters (e.g., modified Y-shaped adapters). A modified end repair method that can improve methylation detection accuracy in dsDNA library preparation based SSM workflows by marking end-repair synthesized bases as methylated is disclosed in International Application No. PCT/US2023/070763, which is incorporated by reference herein in its entirety. This process results in a methylated, end repair ‘scar’ at the 3′ end of a DNA molecule that has high complexity after an epigenetic base conversion process. Because the ‘scar’ is on the 3′ end of the DNA molecule, ligation of the DNA molecules with current/standard NGS adapter topology, after library preparation, can result in the high complexity ‘scar’ being sequenced at the end of read 1 and the start of read 2, e.g., in sequencing by synthesis NGS methods such as those used by Illumina platforms. This does not improve sequencing quality or yield, as the start of read 1 is still of low complexity. See Illumina (2003), What is nucleotide diversity and why is it important?, available at knowledge.illumina.com/instrumentation/general/instrumentation-general-reference_material-list/000001543. Sequence complexity can be important for effective template generation (e.g., in NGS workflows, such as on Illumina sequencing platforms, such as MiSeq and HiSeq 2500 systems) and for the generation of high-quality data. Complexity can be especially important during the first 4-7 cycles of the first sequencing read because the sequencing software can use images from these early cycles to identify the location of each cluster, e.g., during template generation. A cluster is a clonal grouping of template DNA, e.g., bound to the surface of a flow cell. Each cluster is typically seeded by a single, template DNA strand and is clonally amplified until the cluster has approximately 1000 copies. Each cluster (e.g., on the flow cell) produces a single sequencing read. Sequence complexity may also be important for the first 25 cycles in the first sequencing read because this is when phasing/pre-phasing, color matrix corrections, and the pass filter calculations may occur. For example, in Illumina systems, these corrections and calculations can be used in base calling and quality score calculations for all cycles in a run for the clusters that pass filter.

Accordingly, the disclosed methods that use modified end repair and/or modified Y-shaped adapters can provide high quality and high yield sequencing by providing sequence complexity at the start of read 1 as used for cluster quality control (e.g., for the first approximately 25 cycles). In embodiments comprising modified end repair, the high-complexity end-repair scar is sequenced at the start of read 1, thus improving sequencing quality and yield.

In some embodiments, the disclosed methods allow for high quality and high yield sequencing by using Y-shaped adapters (also referred to herein as “complement” Y-shaped adapters) that comprise the reverse complement of the Illumina-specific sequences of the current/standard NGS adapters (as exemplified in FIG. 3). In some embodiments, the ‘top’ strand, i.e., the 5′-connecting strand, of the adapter is modified to include the ‘reverse’/read1 Illumina primer sequence rather than the ‘forward’/read1 Illumina primer sequence. Similarly, the ‘bottom’ strand, i.e., the 3′-connecting strand, of the adapter is modified to include the ‘forward’/read1 Illumina primer binding site, rather than the ‘reverse’/read2 Illumina primer binding site. Molecular barcodes in the adapters can similarly be modified (swapped) in sequence as appropriate. The 3′ T overhang (and/or 3′C overhang in some embodiments) remains on the ‘top’ strand, i.e., the 5′-connecting strand, of the adapter. In embodiments comprising an adapter disclosed herein, read 1 begins at the 3′ end of an adapted DNA molecule. The 3′ end of such a DNA molecule is a high complexity region following an SSM conversion procedure, such as disclosed elsewhere herein. This is in contrast to standard Illumina NGS adapters, wherein read 1 begins at the 5′ end of the molecule, which region may be of low complexity following an SSM conversion procedure. Additionally, as shown in FIG. 3, an exemplary current/standard NGS adapter (left) comprises 25 cytosines in constant regions, whereas an exemplary “complement” Y-shaped adapter of use in the disclosed methods comprises 12 cytosines in constant regions. This approximately 50% reduction in the number of cytosines can reduce 5mC adapter costs, e.g., by approximately 40%.

In some embodiments, library and/or sample-indexing amplification can use the same primers as the disclosed workflows. In some embodiments, a first cycle of library amplification begins with the forward primer, rather than the reverse primer. As this is an SSM workflow, in some embodiments the cytosine bases in the adapters are modified such that they are resistant to a base conversion procedure.

In some embodiments, methods disclosed herein comprise performing a sequencing by synthesis reaction on a converted DNA molecule with a sequencing by synthesis instrument. In embodiments, the converted DNA molecule comprises ligated adapters, a converted region comprising one or more nucleobases that have been converted by a conversion procedure, and a resistant region comprising one or more nucleobases that are resistant to the conversion procedure. In such embodiments, the sequencing by synthesis reaction comprises extending a sequencing primer that binds to the converted DNA molecule upstream of the converted region and the resistant region, calibrating one or more base calling metrics of the sequencing by synthesis instrument based at least in part on data from a region of the converted DNA molecule that is resistant to the conversion procedure, and calling at least a portion of nucleobases in the converted region using the one or more calibrated base calling metrics.

In some embodiments disclosed herein, methods comprise ligating adapters to a DNA molecule, wherein the adapters comprise a resistant region comprising one or more nucleobases that are resistant to a conversion procedure and the DNA molecule comprises one or more nucleobases that are substrates for the conversion procedure. In such embodiments, a conversion procedure is performed on the adapted DNA molecule, producing a converted DNA molecule comprising a converted region that comprises one or more nucleobases that have been converted by the conversion procedure. A sequencing by synthesis reaction is performed on the converted DNA molecule with a sequencing by synthesis instrument, wherein the sequencing by synthesis reaction comprises extending a sequencing primer that binds to the converted DNA molecule upstream of the converted region and the resistant region. One or more base calling metrics of the sequencing by synthesis instrument is calibrated based at least in part on data from the resistant region, and at least a portion of nucleobases in the converted region is called using the one or more calibrated base calling metrics.

In other embodiments, disclosed methods comprise subjecting a DNA molecule comprising one or more nucleobases that are substrates for a conversion procedure to end repair. In such embodiments, the end repair comprises extending a recessed 3′ end of the DNA molecule using a DNA polymerase and deoxyribonucleotides comprising a nucleobase that is resistant to the conversion procedure. The generated end-repaired DNA molecule comprises a resistant region that comprises the nucleobase resistant to the conversion procedure. Adapters are ligated to the end-repaired DNA molecule, and a conversion procedure is performed on the adapted DNA molecule, producing a converted DNA molecule comprising a converted region that comprises one or more nucleobases that have been converted by the conversion procedure. A sequencing by synthesis reaction is performed on the converted DNA molecule with a sequencing by synthesis instrument, wherein the sequencing by synthesis reaction comprises extending a sequencing primer that binds to the converted DNA molecule upstream of the converted region and the resistant region. One or more base calling metrics of the sequencing by synthesis instrument is calibrated based at least in part on data from the resistant region, and at least a portion of nucleobases in the converted region is called using the one or more calibrated base calling metrics.

In some embodiments wherein adapters are ligated to a DNA molecule (such as a converted DNA molecule or an end-repaired DNA molecule), the ligation is a blunt end ligation. In some embodiments, the ligation is a sticky end ligation. In some embodiments, the ligating seals one or more nicks present in the DNA. In particular embodiments, the end repair is performed with a DNA polymerase that does not have 5′-3′ exonuclease activity and/or is not a strand displacing DNA polymerase. In such embodiments, the DNA polymerase is T4 DNA polymerase, T7 DNA polymerase, or Klenow fragment. Ligation of adapters to a DNA molecule of use in a disclosed method can be performed using any appropriate procedure known in the art and/or as disclosed elsewhere herein.

In some embodiments, the DNA to be sequenced in the sequencing by synthesis reaction is cell-free DNA. In some embodiments, the DNA to be sequenced is isolated from a blood sample, e.g., from cells of a blood sample. In some embodiments, DNA to be sequenced is isolated from a tissue sample, such as a tumor sample.

In some embodiments, the methylation levels of DNA (e.g., cell-free DNA or DNA isolated from a sample comprising cells, such as a blood sample) can be used to determine quantities of each of a plurality of cell types from which the DNA originated. This can be useful, e.g., to detect the presence of cancer or precancer, or other conditions (e.g., infection, transplant rejection). In some embodiments, the DNA originated from a tumor cell, e.g., wherein the cancer is a solid tumor cancer or a hematological cancer. In some embodiments, the DNA did not originate from a tumor cell. In some such embodiments, the cancer is not a hematological cancer.

In some such embodiments, the cancer is a solid tumor cancer, e.g., a carcinoma, adenocarcinoma, or sarcoma. Without wishing to be bound by theory, cancers, including solid tumor cancers such as carcinomas, adenocarcinomas, and sarcomas, may cause changes to cell type distribution represented in cfDNA or other samples relative to the cell type distribution in a healthy subject or subject that does not have cancer. See Nabet et al. Cell. 2020, 183:363-376; Watson et al. Sci. Immunol. 2021, 6: eabj8825; Lozano et al. Nature Medicine. 2022, 28:353-362. Such changes may be detected in the methods herein and can be useful in detecting cancer as well as determining cancer prognosis and/or treatment options.

The disclosed methods can be combined with analysis of one or more additional biomarkers. In some embodiments, the disclosed methods are combined with one or more methods, such as but not limited to, methods for assessing DNA methylation patterns, DNA mutations (such as somatic mutations), nucleic acid fragmentation patterns, non-coding RNA (such as micro RNAs (miRNAs), ribosomal RNAs, transfer RNAs, small nucleolar RNAs (snow RNAs), and/or small nuclear RNAs (snRNAs)) levels, and/or cell type proportions/levels, cellular locations, and/or structural modifications of one or more proteins (such as in a sample from a subject). In some embodiments, the disclosed methods are combined with one or more analyses of genetic variations including mutations, rare mutations, indels, rearrangements, copy number variations, transversions, translocations, recombinations, inversion, deletions, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, and/or abnormal changes in nucleic acid 5-methylcytosine.

### B. Adapters

Some embodiments of the disclosed methods use Y-shaped adapters, e.g., that comprise the reverse complement of the Illumina-specific sequences of the current/standard NGS adapters (as exemplified in FIG. 3). As described herein, methods that use a disclosed Y-shaped adapter allow for high quality and high yield sequencing due to maintenance of base diversity at the start of read 1 as used for cluster quality control (e.g., for the first approximately 25 cycles). In some embodiments, adapters (such as the Y-shaped adapters disclosed herein) comprise at least one tag. In some embodiments, the at least one tag comprises a molecular barcode.

In some embodiments of the disclosed methods, the adapter is a Y-shaped adapter that comprises a first strand and a second strand. In some embodiments, the first strand of the Y-shaped adapter comprises a first arm region and a first stem region, and the second strand comprises a second arm region and a second stem region, wherein the second stem region is configured to anneal to the first stem region and the second arm region is configured not to anneal to the first arm region. In some embodiments, the first arm region is located 5′ of the first stem region and the second arm region is located 3′ of the second stem region.

In particular embodiments, the first arm region and second arm region each comprise at least one nucleobase that is resistant to the conversion procedure. In some embodiments, (i) the number of nucleobases with a base-pairing specificity complementary to the nucleobases that are resistant to the conversion procedure in the first arm region is greater than the number of nucleobases that are resistant to the conversion procedure in the first arm region, and/or (ii) the number of nucleobases that are resistant to the conversion procedure in the first arm region is less than 25% of the number of nucleobases in the first arm region. In such embodiments, (i) the number of nucleobases with a base-pairing specificity complementary to the nucleobases that are resistant to the conversion procedure in the second arm region is greater than the number of nucleobases that are resistant to the conversion procedure in the second arm region and/or (ii) the number of nucleobases that are resistant to the conversion procedure in the second arm region is less than 25% of the number of nucleobases in the second arm region.

In particular embodiments, the adapter is Y-shaped oligonucleotide adapter comprising first and second strands, wherein (a) the first strand comprises a first arm region and a first stem region; (b) the second strand comprises a second arm region and a second stem region, wherein the second stem region is configured to anneal to the first stem region and the second arm region is configured not to anneal to the first arm region;(c) the first arm region and second arm region each comprise one or more modified nucleobases that are resistant to a conversion procedure; (d) (i) the number of nucleobases with a base-pairing specificity complementary to the modified nucleobases that are resistant to the conversion procedure in the first arm region is greater than the number of modified nucleobases that are resistant to the conversion procedure in the first arm region, and/or (ii) the number of modified nucleobases that are resistant to the conversion procedure in the first arm region is less than 25% of the number of nucleobases in the first arm region; and (e)(i) the number of nucleobases with a base-pairing specificity complementary to the modified nucleobases that are resistant to the conversion procedure in the second arm region is greater than the number of modified nucleobases that are resistant to the conversion procedure in the second arm region and/or (ii) the number of modified nucleobases that are resistant to the conversion procedure in the second arm region is less than 25% of the number of nucleobases in the second arm region.

In some embodiments, the nucleobase that is resistant to the conversion procedure comprises a modified nucleobase, such as 4-methylcytosine (4mC), 5-methylcytosine (5mC), 5-hydroxymethylcytosine (5hmC), N6-methyladenosine (6 mA), bromodeoxyuridine (BrdU), 8-oxoguanine (8oxoG), 5-pyrrolo cytosine, 5-glucoylhydroxymethylated (5-ghmC), 5-caryboxylcytosine (5-caC), and/or 5-propynyl cytosine. In particular embodiments, the nucleobase that is resistant to the conversion procedure is a modified cytosine. In some particular embodiments, the modified cytosine is 5-methylcytosine. In other particular embodiments, the modified cytosine is 5-hydroxymethylcytosine. In some embodiments, the first arm region, the second arm region, the first stem region, and/or the second stem region comprise one or more modified cytosines, such as 5-methylcytosine, 5-hydroxymethylcytosine, 5-caryboxylcytosine (5-caC), and/or 5-propynyl cytosine.

In some embodiments, the adapter is a Y-shaped oligonucleotide adapter comprising first and second strands, wherein (a) the first strand comprises a first arm region and a first stem region; (b) the second strand comprises a second arm region and a second stem region, wherein the second stem region is configured to anneal to the first stem region and the second arm region is configured not to anneal to the first arm region; (c) the first arm region and second arm region each comprise modified cytosines; (d)(i) the number of guanines in the first arm region is greater than the number of modified cytosines in the first arm region, and/or (ii) the number of modified cytosines in the first arm region is less than 25% of the number of nucleobases in the first arm region; and (e)(i) the number of guanines in the second arm region is greater than the number of modified cytosines in the second arm region and/or (ii) the number of modified cytosines in the second arm region is less than 25% of the number of nucleobases in the second arm region. In particular embodiments, the modified cytosine comprises 4-methylcytosine (4mC), 5-methylcytosine (5mC), 5-hydroxymethyl-cytosine (5hmC), 5-pyrrolo cytosine, 5-glucoylhydroxymethylated (5-ghmC), 5-caryboxylcytosine (5-caC), and/or 5-propynyl cytosine.

In some embodiments, the modified cytosine is 5-methylcytosine. In other embodiments, the modified cytosine is 5-hydroxymethylcytosine. In particular embodiments, the first arm region, the second arm region, the first stem region, and/or the second stem region of the adapter comprise one or more modified cytosines, optionally wherein the one or more modified cytosines are 5-methylcytosine, 5-hydroxymethylcytosine, 5-caryboxylcytosine (5-caC), and/or 5-propynyl cytosine. In some embodiments, at least 50%, at least 60%, at least 70%, at least 75%, at least 80%, at least 85%, at least 90%, or at least 95% of the cytosines in the first arm region, the second arm region, the first stem region, and/or the second stem region are modified cytosines. In particular embodiments, the first arm region, the second arm region, the first stem region, and/or the second stem region are substantially free of unmodified cytosines. In some embodiments, the first arm region, the second arm region, the first stem region, and/or the second stem region are at least 90% free of unmodified cytosines, such as 91%, 92%, 93%, 94%, 95%, 96%, 97%, 98%, 99%, or 100% free of unmodified cytosines.

In some disclosed embodiments, the resistant region is at least about 10, at least about 11, at least about 12, at least about 13, at least about 14, at least about 15, at least about 16, at least about 17, at least about 18, at least about 19, or at least about 20 nucleotides in length. In some embodiments, the resistant region is about 10-40, about 10-35, about 10-30, about 10-25, about 10-20, about 10-15, about 15-40, about 15-35, about 15-30, about 15-25, about 15-20, about 20-40, about 20-35, about 20-30, or about 20-25 nucleotides in length. In some embodiments, the resistant region is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length.

In some embodiments, the resistant region is located 3′ of the converted region. In some embodiments, the resistant region comprises at least 2, at least 3, at least 4, at least 5, at least 6, at least 7, at least 8, at least 9, at least 10, at least 11, at least 12, at least 13, at least 14, or at least 15 nucleobases that are resistant to the conversion procedure. In some embodiments, the resistant region comprises 2-30, 2-25, 2-20, 2-15, 2-10, 2-9, 2-8, 2-7, 2-6, 2-5, 2-4, or 2-3 nucleobases that are resistant to the conversion procedure. In particular embodiments, the resistant region comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, of 40 nucleobases that are resistant to the conversion procedure.

### C. Sequencing

In general, sample nucleic acids flanked by adapters with or without prior amplification can be subject to sequencing. Sequencing methods include sequencing by synthesis, such as next generation sequencing (NGS) (such as Illumina NGS) or Single Molecule Sequencing by Synthesis (SMSS) (Helicos). Sequencing reactions can be performed in a variety of sample processing units, which may include multiple lanes, multiple channels, multiple wells, or other means of processing multiple sample sets substantially simultaneously. Sample processing unit can also include multiple sample chambers to enable processing of multiple runs simultaneously.

In embodiments of the disclosed methods, the sequencing comprises a sequencing by synthesis reaction. In a sequencing by synthesis reaction, the sequence of a DNA molecule is determined by detecting the incorporation of each nucleotide into a complementary strand synthesized by a DNA polymerase. As the polymerase synthesizes a copy of a single strand of DNA, the incorporation of each nucleotide is monitored, such as by detection of fluorescently labeled nucleotides.

In some embodiments, the sequencing by synthesis reaction comprises extending a sequencing primer that binds to the converted DNA molecule upstream of a converted region and a resistant region in a converted DNA molecule comprising ligated adapters; (b) calibrating one or more base calling metrics of the sequencing by synthesis instrument based at least in part on data from the resistant region, thereby providing one or more calibrated base calling metrics; and (c) calling at least a portion of nucleobases in the converted region using the one or more calibrated base calling metrics. In some embodiments, the sequencing by synthesis reaction comprises sequencing the DNA in a manner that distinguishes the first nucleobase from the second nucleobase. In some embodiments, the sequencing by synthesis reaction comprises next generation sequencing. In particular embodiments, the sequencing by synthesis reaction comprises generating a plurality of sequencing reads and mapping the plurality of sequencing reads to one or more reference sequences to generate mapped sequence reads. In some embodiments, the one or more base calling metrics comprises clusters passing filter, phasing/pre-phasing, and/or color matrix corrections values.

In some embodiments, sequence coverage of the genome may be, for example, less than 5%, 10%, 15%, 20%, 25%, 30%, 40%, 50%, 60%, 70%, 80%, 90%, 95%, 99%, 99.9% or 100%. In some embodiments, the sequence reactions may provide for sequence coverage of, for example, at least 5%, 10%, 15%, 20%, 25%, 30%, 40%, 50%, 60%, 70%, or 80% of the genome. Sequence coverage can performed on, for example, at least 5, 10, 20, 70, 100, 200 or 500 different genes, or up to, for example, 5000, 2500, 1000, 500 or 100 different genes.

Simultaneous sequencing reactions may be performed using multiplex sequencing. In some cases, cell-free nucleic acids may be sequenced with at least, for example, 1000, 2000, 3000, 4000, 5000, 6000, 7000, 8000, 9000, 10000, 50000, or 100,000 sequencing reactions. In other cases cell-free nucleic acids may be sequenced with less than, for example, 1000, 2000, 3000, 4000, 5000, 6000, 7000, 8000, 9000, 10000, 50000, or 100,000 sequencing reactions.

Sequencing reactions may be performed sequentially or simultaneously. Subsequent data analysis may be performed on all or part of the sequencing reactions. In some cases, data analysis may be performed on at least, for example, 1000, 2000, 3000, 4000, 5000, 6000, 7000, 8000, 9000, 10000, 50000, or 100,000 sequencing reactions. In other cases, data analysis may be performed on less than, for example, 1000, 2000, 3000, 4000, 5000, 6000, 7000, 8000, 9000, 10000, 50000, or 100,000 sequencing reactions. An exemplary read depth is 1000-50000 or 1000-10000 or 1000-20000 reads per locus (base).

In general, sequencing of epigenetic target regions, e.g., to analyze a modified nucleoside profile of DNA, requires a lesser depth of sequencing than sequencing of a sequence-variable target region, e.g., for analysis of mutations. Hence, lesser sequencing depths may in some cases be adequate for the methods described herein.

### 1. Differential Depth of Sequencing

In some embodiments, nucleic acids corresponding to the sequence-variable target region set are sequenced to a greater depth of sequencing than nucleic acids corresponding to the epigenetic target region set. In some embodiments, nucleic acids corresponding to the hydroxymethylation-variable target region set are sequenced to a greater depth of sequencing than nucleic acids corresponding to at least one other target region set. For example, the depth of sequencing for nucleic acids corresponding to the sequence-variable and/or hydroxymethylation-variable target region sets may be at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold greater, or 1.25- to 1.5-, 1.5- to 1.75-, 1.75- to 2-, 2- to 2.25-, 2.25- to 2.5-, 2.5- to 2.75-, 2.75- to 3-, 3- to 3.5-, 3.5- to 4-, 4- to 4.5-, 4.5- to 5-, 5- to 5.5-, 5.5- to 6-, 6- to 7-, 7- to 8-, 8- to 9-, 9- to 10-, 10- to 11-, 11- to 12-, 13- to 14-, 14- to 15-fold, or 15- to 100-fold greater, than the depth of sequencing for nucleic acids corresponding to the epigenetic target region set or to at least one other target region set. In some embodiments, said depth of sequencing is at least 2-fold greater. In some embodiments, said depth of sequencing is at least 5-fold greater. In some embodiments, said depth of sequencing is at least 10-fold greater. In some embodiments, said depth of sequencing is 4- to 10-fold greater. In some embodiments, said depth of sequencing is 4- to 100-fold greater. Each of these embodiments refer to the extent to which nucleic acids corresponding to the sequence-variable target region set are sequenced to a greater depth of sequencing than nucleic acids corresponding to the epigenetic target region set.

In some embodiments, the captured cfDNA corresponding to the sequence-variable target region set and the captured cfDNA corresponding to the epigenetic target region set are sequenced concurrently, e.g., in the same sequencing cell (such as the flow cell of an Illumina sequencer) and/or in the same composition, which may be a pooled composition resulting from recombining separately captured sets or a composition obtained by capturing the cfDNA corresponding to the sequence-variable target region set and the captured cfDNA corresponding to the epigenetic target region set in the same vessel.

In some embodiments, the captured cfDNA corresponding to the hydroxymethylation variable target region set and the captured cfDNA corresponding to the at least one other target region set are sequenced concurrently, e.g., in the same sequencing cell (such as the flow cell of an Illumina sequencer) and/or in the same composition, which may be a pooled composition resulting from recombining separately captured sets or a composition obtained by capturing the cfDNA corresponding to the hydroxymethylation variable target region set and the captured cfDNA corresponding to the at least one other target region set in the same vessel.

### Analysis

Defining Regions of the End-Repaired DNA that were Synthesized During the End Repair

In general, the methods described herein rely on the use of at least one dNTP comprising a modified base in the end repair reaction coupled with the use of modification-sensitive sequencing which can detect the modified base. This allows for regions synthesized in the end repair to be identified in the sequencing data. This is important because these synthesized regions can lead to artifactual data in typical sequencing reactions which do not control for these synthesized regions. For example, when end repair is performed with unmodified dNTPs prior to methylation-sensitive sequencing (e.g., bisulfite sequencing), the end repair can lead to 5′ overhang filling, nick translation and gap filling with dCTP comprising unmodified cytosines. These unmodified cytosines may not reflect the original methylation status at these positions in the original DNA molecule (i.e., prior to the formation of the overhang, nicks and gaps) and thus the end repair can lead to artifactual methylation information. The methods disclosed herein avoid such artifactual information by identifying the sequencing data corresponding to these synthesized regions. Such regions can, e.g., then be filtered such that they are not used to classify the methylation status of the DNA molecule.

The regions synthesized in the end repair can be classified in a variety of ways and the exact approach will depend on the identity of the modified base used in the end repair reaction as well as the modification sensitive sequencing method being used. Moreover, the exact end points of the regions classified as being synthesized during end repair can be determined by the user. The basic step of the identification of the regions synthesized during the end repair reaction is the identification of the presence of the base modification in the at least one type of dNTP used in the end repair reaction.

In some embodiments, the end repair is performed with dNTP comprising 5mC or 5hmC. 5mC and 5hmC are both naturally occurring base modifications, so upon the identification of these modified bases in the sequencing data may derive from: (i) modified bases present in the original DNA molecule; or (ii) modified bases introduced in the end repair reaction. 5mC and 5hmC can, however, be classified as being introduced in the end repair reaction when they occur in a non-CpG sequence context. While CpH (i.e., CpA, CpT, CpC) methylation has been described in humans, it is thought to comprise 0.02% of total methyl-cytosine in differentiated somatic cells (Jang et al. Genes (Basel). 2017 June; 8(6): 148). As such, methylated cytosines in a CpH sequence context can confidently be attributed to regions synthesized during end repair. This is particularly the case when the disclosed methods comprise enrichment for a sequence panel wherein the panel does not comprise regions known to contain methylated CpH sites. Alternatively, the classification of whether or not a methylated CpH is part of a synthesized region can be made by accounting for: (i) the position of particular of the CpH site in a reference sequence; and/or (ii) the methylation status of the surrounding CpH sites. For example, if a CpH site is known to be methylated in nature (e.g., by comparison to reference data), then methylation detected at that CpH site can be ignored when defining regions synthesized during end repair. Similarly, detected methylation at such CpH sites can be called as the true methylation status in the DNA sample. If a CpH site known to be methylated in nature is detected as being methylated in the sequencing data, but is contained within a string of other methylated CpH sites, some of which are not known to be methylated in nature (e.g., by comparison to reference data), the region may still be classified as being synthesized during end repair.

In embodiments wherein the end repair is performed with dNTP comprising 5mC and/or 5hmC, a region of the one or more regions of the end-repaired DNA that were synthesized during the end repair is defined as: (i) the sequence between two non-methylated cytosines which span a methylated non-CpG cytosine; and/or (ii) the sequence between a non-methylated cytosine and the end of a sequence read wherein there is no additional non-methylated cytosine between the non-methylated cytosine and the end of the sequence read. Alternatively, the one or more regions of the end-repaired DNA that were synthesized during the end repair may be defined as: (i) the sequence from a first methylated non-CpG cytosine to the last methylated non-CpG cytosines in one or more consecutive methylated non-CpG cytosines; and/or (ii) the sequence from a methylated cytosine (5mC or 5hmC) in a non-CpG context to the end of a sequence read wherein there is no non-methylated cytosine between the methylated cytosine in the non-CpG context and the end of the sequence read. The “end of the sequence read” refers to the portion of the sequence read which corresponds to the end-repaired DNA molecule and does not include, e.g. adapter sequences.

In some embodiments the end repair is performed with dNTPs comprising base modifications which are not naturally found in the subject the DNA sample derives from, or are present at only very low frequencies. For example 4mC does not occur in mammals (e.g. humans), whereas 6 mA occurs only at very low frequencies (Xiao et al. Molecular Cell Volume 71, Issue 2, 19 Jul. 2018, Pages 306-318.e7). In these embodiments, the regions of the end-repaired DNA that were synthesized during the end repair can be classified simply as any region wherein the modified base is detected. While such an approach may result in falsely classifying naturally occurring low frequency base modifications as being the result of end repair, this will be rare and may simply result in the corresponding sequence data not being used for further analysis. This is preferable to using sequence data from regions synthesized during end repair, which may contain artifactual data which may lead to false inferences regarding the corresponding DNA sample and subject.

Therefore, in some embodiments wherein the modified base is other than 5mC or 5hmC, a region of the one or more regions is defined as: (i) the sequence between two non-modified bases spanning a modified base, wherein the bases are of the same identity as the bases present in the at least one type of dNTP comprising the modified base; and/or (ii) the sequence between a non-modified base and the end of a sequence read, wherein there is no additional non-modified bases between the non-modified base and the end of the sequence read, where the non-modified bases are of the same identity as the modified base present in the at least one type of dNTP comprising the modified base. Alternatively, the one or more regions of the end-repaired DNA that were synthesized during the end repair may be defined as: (i) the sequence from a first modified to the last modified base in one or more consecutive modified bases wherein the bases are of the same identity as the bases present in the at least one type of dNTP comprising the modified base; and/or (ii) the sequence from a modified base to the end of a sequence read wherein there is no non-modified base between the modified base and the end of the sequence read where the modified base and non-modified base are of the same identity to the at least one type of dNTP comprising the modified base.

Once identified, the regions of the end-repaired DNA classified as being synthesized during the end repair may be filtered out of the sequence data such that they are not used for further analysis, such as variant calling or for determining the modification status of bases in the original DNA molecule (i.e., prior to end repair). Accordingly, in some embodiments the methods disclosed herein further comprise analyzing at least some of the sequence data corresponding to regions that are not identified as being synthesized during the end repair to detect the presence or absence of base modifications or mutations present in the DNA sample. The disclosed methods of identifying regions synthesized during end repair are advantageous over the prior art methods which use uninformed “end-clipping” because these prior art methods potentially remove regions which were not synthesized in the end repair reaction and are thus representative of the original DNA molecule.

### D. End Repair and A-Tailing

In some embodiments of the disclosed methods, a DNA molecule comprising one or more nucleobases that are substrates for a conversion procedure is subjected to end repair. In some embodiments, the end repair comprises extending a recessed 3′ end of the DNA molecule using a DNA polymerase and deoxyribonucleotides comprising a nucleobase that is resistant to the conversion procedure, thereby generating an end-repaired DNA molecule comprising a resistant region that comprises the nucleobase resistant to the conversion procedure. In particular embodiments, the end repair is performed with a DNA polymerase that does not have 5′-3′ exonuclease activity and/or is not a strand displacing DNA polymerase. In such embodiments, the DNA polymerase is T4 DNA polymerase, T7 DNA polymerase, or Klenow fragment. Some embodiments of the disclosed methods further comprise performing an A-tailing reaction.

End repair refers to methods for repairing DNA by the conversion of non-blunt ended DNA into blunt ended DNA. Sequencing workflows typically use end repair to make ends of DNA molecules compatible with adapters, which are subsequently ligated onto the DNA. Fragmented and/or damaged DNA (e.g., cfDNA or DNA from FFPE samples) often contain non-blunt ends, which contain 3′ overhangs and/or 5′ overhangs. A 3′ overhang refers to the 3′ end of a DNA strand which extends beyond the 5′ end of the paired strand, resulting in one or more unpaired nucleotides at the 3′ end of the DNA strand. Conversely, a 5′ overhang refers to the 5′ end of a DNA strand which extends beyond the 3′ end of the paired strand, resulting in one or more unpaired nucleotides at the 5′ end of the DNA strand.

The process of end repair involves the conversion of double-stranded DNA with 3′ overhangs and/or 5′ overhangs to double-stranded DNA without overhangs. This can be done using an enzyme such as T4 DNA polymerase and/or Klenow fragment. The 3′ to 5′ exonuclease activity of these enzymes removes the 3′ ends at 3′ overhangs and the 5′ to 3′ polymerase activity of these enzymes extends the 3′ ends at 5′ overhangs to remove the 5′ overhang, thereby generating a blunt-ended DNA molecule. In order to fill in these 5′ overhangs, end repair is conducted in the presence of dATP, dCTP, dGTP and dTTP. End repair can also include a second step, which involves the addition of a phosphate group to the 5′ ends of DNA, by an enzyme such as polynucleotide kinase. This makes the 5′ ends of the end-repaired DNA molecules compatible with the subsequent action of DNA polymerases and DNA ligases.

As used herein, the term “A-tailing” refers to the addition of a single deoxyadenosine residue to the end of a blunt-ended double-stranded DNA fragment to form a 3′ deoxyadenosine single-base overhang. Such A tailing reactions are conducted with polymerases that have the ability to add a non-templated A to the 3′ end of a blunt, double-stranded DNA molecule. Polymerases capable of A-tailing typically do not possess 3′-5′ exonuclease activity. When A-tailing is performed as a separate reaction to end repair, it can be conducted in the presence of dATP, but the absence of dCTP, dTTP and dGTP. A-tailed fragments are not compatible for self-ligation (i.e., self-circularizatian and concantenation of the DNA), but they are compatible with 3′ T-overhangs, which can be used on adapters. Methods comprising end repair, A-tailing, and ligation to adapters with 3′ T-overhangs can result in higher efficiency ligation, compared to blunt ended ligation, as blunt ligation can lead to self-ligation of the adapters and/or DNA molecules.

In some cases, the methods disclosed herein comprise end repair of the DNA molecules followed by blunt end ligation of adapters. In other cases, the methods disclosed herein comprise end repair of the DNA molecules followed by A-tailing and sticky-end ligation of T-tailed adapters. When the methods disclosed herein comprise an A-tailing step, it may be performed separately from the end repair with an intervening reaction clean-up step or it may be performed in the same reaction as the end repair (e.g. using NEBNext® Ultra™ II End Repair/dA-Tailing Module (E7546)). Accordingly, in some embodiments, the end-repair and the A-tailing reaction are performed in the same reaction mixture, optionally wherein the end-repair and the A-tailing reaction are performed a single tube and/or optionally wherein the end-repair and the A-tailing reaction are performed without an intervening clean-up step. In instances wherein the A-tailing reaction is performed in the same reaction as end repair, a sticky-end ligation may be performed with a mixture of T-tailed adapters and C-tailed adapters.

In some embodiments, the end-repair and the A-tailing reactions are performed in a single tube. In such cases, the A tailing reaction can be performed at a higher temperature than the end repair. In some embodiments, the A tailing reaction is performed at a higher temperature than the end repair, optionally wherein the end repair is performed at about 15-35 °C and/or the A tailing is performed at a temperature over about 60 °C, further optionally wherein the temperature over 60 °C is about 60 °C-75 °C. Optionally, end repair is performed at ambient temperature (e.g. 15-35 °C) and A tailing is performed at a temperature over 60 °C. The A tailing reaction can be performed using a thermostabile polymerase (e.g., Taq DNA polymerase, Tfl DNA polymerase, Bst DNA Polymerase, Large Fragment or Tth DNA polymerase) and the method further comprises increasing temperature of the sample after the end repair to inactivate the polymerase used in end repair (e.g., T4 DNA polymerase or Klenow fragment). In some embodiments the A-tailing is performed using a DNA polymerase that: (i) does not possess 5′-3′ exonuclease activity; and/or (ii) is not a strand displacing DNA polymerase. These properties reduce the ability of the DNA polymerase to extend from a nick. This reduces the level of synthesis which may occur during the end repair and A-tailing reactions thus reducing the proportion of sequencing data that may be filtered out as potentially containing artifactual data. Accordingly, in some embodiments, the A-tailing is performed using a DNA polymerase that cannot extend from a nick in the DNA, such as HemoKlen Taq. In some embodiments, a DNA polymerase that does not possess 5′-3′ exonuclease activity and/or is not a strand displacing DNA polymerase, such as HemoKlen Taq. such as HemoKlen Taq. In other embodiments, the A-tailing is performed using Taq DNA polymerase. In other embodiments, the A-tailing is performed using Tfl polymerase, Bst DNA Polymerase, Large Fragment or Tth polymerase.

In some embodiments of the methods disclosed herein the end repair is performed with a polymerase which lacks 5′ to 3′ exonuclease activity and/or strand displacement activity. In some cases, the polymerase used in the end repair reaction may be Q5@High-Fidelity DNA Polymerase, Q5U@Hot Start High-Fidelity DNA Polymerase, Phusion® High-Fidelity DNA Polymerase, Hemo KlenTaq, phi29 DNA Polymerase, T7 DNA Polymerase, DNA Polymerase I (E. coli), DNA Polymerase I, Large (Klenow) Fragment (“Klenow fragment”) or T4 DNA Polymerase. In some embodiments, the polymerase used in the end repair is T4 DNA Polymerase or Klenow fragment.

In some embodiments, the methods disclosed herein comprise an A tailing reaction after the end repair and before the ligation reaction, wherein the end repair and A tailing reactions are separated by a reaction cleanup. The A tailing reaction is typically performed in the presence of dATP, but in the absence of dCTP, dTTP and dGTP. In some embodiments, the A tailing reaction is performed using Klenow Fragment lacking 3′-5′ exonuclease activity.

The dNTP that comprises a modified base may comprise any modified base wherein the presence or the absence of the modification can be detected by a type of modification sensitive sequencing. The modified base may be 4-methylcytosine (4mC), 5-methylcytosine (5mC), 5-hydroxymethyl-cytosine (5hmC), N6-methyladenosine (6 mA), bromodeoxyuridine (BrdU), 5-fluorodeoxyuridine (FldU), 5-iododeoxyuridine (IdU), 5-ethynyldeoxyuridine (EdU) and/or 8-oxoguanine (8oxoG).

When a dNTP comprising a modified base is used, it may be used in place of the equivalent unmodified base in the end repair reaction. For instance, if a dCTP comprising 5mC is used in the end repair reaction, there may be no dCTP comprising an unmodified cytosine. This would ensure that dCTPs incorporated into the DNA molecule during the end repair reaction contain 5mC. In some embodiments, multiple types of dNTP comprising a modified base are used in the end repair. For example, dATP comprising 6 mA and dCTP comprising 5mC can be used in the end repair reaction in place of dATP comprising unmodified adenine and dCTP comprising unmodified cytosine. The use of multiple types of dNTP comprising a modified base is advantageous because it provides increased resolution in defining the regions of the end-repaired DNA molecule which have been synthesized during the end repair reaction. This is because, in this example, the end of a synthesized region can be defined as the first unmodified adenine or unmodified cytosine after a stretch of containing 6mAs and/or 5mCs, rather than relying on the detection of solely an unmodified adenine or solely an unmodified cytosine.

The modification sensitive sequencing method used will depend on the type of modified base used in the end-repair reaction such that the specific modification can be detected. Exemplary conversion-based methods are described above alongside the base modification which they can detect. Moreover, nanopore-based sequencing can be used to detect 4mC, 5mC, 5hmC, 6 mA, BrdU, FdU, IdU, and EdU, and single-molecule real time (SMRT) sequencing from Pacific Biosciences can be used to detect 4mC, 5mC, 5hmC, 6 mA, and 8oxoG.

### E. Adapter Ligation

Some embodiments of the disclosed methods comprise ligating an adapter (such as a modified Y-shaped adapter as disclosed herein) to the DNA. In embodiments comprising an end repair step, once the DNA has been end-repaired it can subjected to blunt-end ligation with blunt-ended adapters, in cases where A-tailing is not performed, or sticky end ligation with T-tailed adapters, when A tailing is performed. DNA molecules can be ligated to adapters at either one end or both ends. DNA molecules can be ligated with at least partially double stranded adapter (e.g., a Y-shaped or bell-shaped adapter). In embodiments wherein the modification-sensitive sequencing comprises a conversion procedure, the ligation step can take place before or after the conversion step. In some embodiments, the ligation step is performed after the conversion step.

DNA ligase and adapters are added to ligate DNA molecules in the sample with an adapter on one or both ends, i.e., to form adapted DNA. An adapter is typically a short nucleic acid (e.g., less than about 500, less than about 100 or less than about 50 nucleotides in length, or about 20-30, 20-40, 30-50, 30-60, 40-60, 40-70, 50-60, 50-70, 20-500, or 30-100 bases from end to end) that are typically at least partially double-stranded and can be ligated to the end of a given sample DNA molecule. In some instances, two adapters can be ligated to a single sample DNA molecule, with one adapter ligated to each end of the sample nucleic acid molecule.

In some embodiments, the ligase used in ligation reactions can act on both single strand DNA nicks and double stranded DNA ends. In some cases, the ligase is T4 DNA ligase or T3 DNA ligase. Adapters can include nucleic acid primer binding sites to permit amplification of a sample DNA molecule flanked by adapters at both ends, and/or a sequencing primer binding site, including primer binding sites for sequencing applications, such as various next generation sequencing (NGS) applications. Adapters can include a sequence for hybridizing to a solid support, e.g., a flow cell sequence. Adapters can also include binding sites for capture probes, such as an oligonucleotide attached to a flow cell support or the like. Adapters can also include sample indexes and/or molecular barcodes. These are typically positioned relative to amplification primer and sequencing primer binding sites, such that the sample index and/or molecular barcode is included in amplicons and sequencing reads of a given DNA molecule. Adapters of the same or different sequence can be linked to the respective ends of a sample DNA molecule. In some cases, adapters of the same or different sequence are linked to the respective ends of the DNA molecule except that the sample index and/or molecular barcode differs in its sequence. In some embodiments, the adapter is a Y-shaped adapter in which one end is blunt ended or tailed as described herein, for joining to a nucleic acid molecule, which is also blunt ended or tailed with one or more complementary nucleotides to those in the tail of the adapter. In another exemplary embodiment, an adapter is a bell-shaped adapter that includes a blunt or tailed end for joining to a DNA molecule to be analyzed. Other exemplary adapters include T-tailed, C-tailed or hairpin shaped adapters. For example, a hairpin shaped adaptor can comprise a complementary double stranded portion and a loop portion, where the double stranded portion can be attached (e.g., ligated) to a double-stranded polynucleotide. Hairpin shaped sequencing adaptors can be attached to both ends of a polynucleotide fragment to generate a circular molecule, which can be sequenced multiple times. The adapters used in the methods of the present disclosure comprise one or more known modified nucleosides, such as methylated nucleosides. In instances where two adapters are ligated to a sample nucleic acid (one at each end), either or both of the adapters may comprise one or more known modified nucleosides. Typically, the primer binding site(s), sequencing primer binding site(s), sample index(es) and/or molecular barcode(s), if present, do not comprise the known modified nucleosides that change base pairing specificity as a result of the conversion procedure.

In some embodiments, adapters may be added to the DNA or a subsample thereof. Adapters can be ligated to DNA at any point in the methods herein. In some embodiments, adapters are ligated to the DNA of a sample or subsample thereof prior to annealing primers to the DNA for capture probe generation. In some such embodiments, the adapter-ligated DNA is amplified prior to annealing primers to the DNA for capture probe generation. In some embodiments, adapters are ligated to the DNA of a sample or subsample thereof before the DNA is contacted with the capture probes. In some embodiments, the DNA to which the adapters are ligated is in the same sample or subsample as the DNA used as a template to generate capture probes. In some embodiments, the DNA to which the adapters are ligated is in a different sample or subsample, e.g., a second sample or a second subsample of a first sample, than the DNA used as a template to generate capture probes. In some embodiments, the adapters ligated to DNA captured by the capture probes.

In some embodiments, the primers used to generate capture probes are not complementary to adapters, and the resulting capture probes therefore do not comprise adapters. Adapter-ligated DNA can therefore be selectively amplified in the presence of capture probes that do not comprise adapters. Similarly, adapter-ligated DNA can be separated from DNA that does not comprise adapters.

In some embodiments, the disclosed methods comprise analyzing DNA in a sample. In such methods, adapters may be added to the DNA. This may be done concurrently with an amplification procedure, e.g., by providing the adapters in a 5′ portion of a primer (where PCR is used, this can be referred to as library prep-PCR or LP-PCR), before, or after an amplification step. In some embodiments, adapters are added by other approaches, such as ligation. In some such methods, first adapters are added to the 3′ ends of the nucleic acids by ligation, which may include ligation to single-stranded DNA. In some such methods, first adapters are added to the 5′ ends of the nucleic acids by ligation, which may include ligation to single-stranded DNA. In some embodiments, prior to any partitioning or capturing steps, first adapters are added to the nucleic acids by ligation, which may include ligation to single-stranded DNA (e.g., to the 3′ ends thereof). In some embodiments, the capture probes can be isolated after partitioning and ligation. For example, the hypomethylated partition can be ligated with adapters and a portion of the ligated hypomethylated partition can then be used to generate the capture probes for rearrangements. The adapter can be used as a priming site for second-strand synthesis, e.g., using a universal primer and a DNA polymerase. A second adapter can then be ligated to at least the 3′ end of the second strand of the now double-stranded molecule. In some embodiments, the first adapter comprises an affinity tag, such as biotin, and nucleic acid ligated to the first adapter is bound to a solid support (e.g., bead), which may comprise a binding partner for the affinity tag such as streptavidin. For further discussion of a related procedure, see Gansauge et al., Nature Protocols 8:737-748 (2013). Commercial kits for sequencing library preparation compatible with single-stranded nucleic acids are available, e.g., the Accel-NGS® Methyl-Seq DNA Library Kit from Swift Biosciences. In some embodiments, after adapter ligation, nucleic acids are amplified.

In some embodiments, the single-stranded DNA library preparation is performed in a one-step combined phosphorylation/ligation reaction, e.g., as described in Troll et al., BMC Genomics, 20:1023 (2019), available at doi.org/10.1186/si2864-019-6355-0. This method, called Single Reaction Single-stranded LibrarY (“SRSLY,”) can be performed without end-polishing. SRSLY may be useful for converting short and fragmented DNA molecules, e.g., cfDNA fragments, into sequencing libraries while retaining native lengths and ends. The SRSLY method can create sequencing libraries (e.g., Illumina sequencing libraries) from fragmented or degraded template (input) DNA. In particular embodiments, template DNA is first heat denatured and then immediately cold shocked to render the template DNA molecules single-stranded. The DNA can be maintained as single-stranded throughout the ligation reaction by the inclusion of a thermostable single-stranded binding protein (SSB). Next, the template DNA, which at this point can be single-stranded and coated with SSB, is placed in a phosphorylation/ligation dual reaction with directional dsDNA NGS adapters that contain single-stranded overhangs. Both the forward and reverse sequencing adapters can share similar structures but differ in which termini is unblocked in order to facilitate proper ligations. Both sequencing adapters can comprise a dsDNA portion and a single-stranded splint overhang of random nucleotides that occurs on the 3-prime terminus of the bottom strand of the forward adapter and the 5-prime terminus of the bottom strand of the reverse adapter. In this way, the forward adapter (e.g., (P5) Illumina adapter) can delivered to the 5-prime end of template molecules and the reverse adapter (e.g., (P7) Illumina adapter) is delivered to the 3-prime end of template molecules. Thus, the native polarity of input DNA molecules can be retained.

During the dual phosphorylation/ligation reaction, T4 Polynucleotide Kinase (PNK) can be used to prepare template DNA termini for ligation by phosphorylating 5-prime termini and dephosphorylating 3-prime termini. T4 PNK works on both ssDNA and dsDNA molecules and has no activity on the phosphorylation state of proteins. Simultaneously, the random nucleotides of the splint adapter can be annealed to the single-stranded template molecule. This creates a short, localized dsDNA molecule, enabling ligation of template to adapter with a ligase such as T4 DNA ligase, which has high ligation efficiency on dsDNA templates but low efficiency on ssDNA. After the single phosphorylation/ligation reaction is complete, the library DNA can be, e.g., purified and placed directly into standard NGS indexing PCR, compatible with both traditional single or dual index primers.

In some embodiments, the adapters include different tags of sufficient numbers that the number of combinations of tags results in a low probability e.g., 95, 99 or 99.9% of two nucleic acids with the same start and stop points receiving the same combination of tags. Adapters, whether bearing the same or different tags, can include the same or different primer binding sites, but preferably adapters include the same primer binding site.

In some embodiments, following attachment of adapters, the nucleic acids are subject to amplification. The amplification can use, e.g., universal primers that recognize primer binding sites in the adapters.

In some embodiments, following attachment of adapters, the DNA or a subsample or portion of the DNA is partitioned, comprising contacting the DNA with an agent that preferentially binds to nucleic acids bearing an epigenetic modification. The nucleic acids are partitioned into at least two partitioned subsamples differing in the extent to which the nucleic acids bear the modification from binding to the agents. For example, if the agent has affinity for nucleic acids bearing the modification, nucleic acids overrepresented in the modification (compared with median representation in the population) preferentially bind to the agent, whereas nucleic acids underrepresented for the modification do not bind or are more easily eluted from the agent. The nucleic acids can then be amplified from primers binding to the primer binding sites within the adapters. Partitioning may be performed instead before adapter attachment, in which case the adapters may comprise differential tags that include a component that identifies which partition a molecule occurred in.

In some embodiments, the nucleic acids are linked at both ends to Y-shaped adapters including primer binding sites and tags. The molecules are amplified.

### F. Molecular Tags; Tagging

In some embodiments, the DNA molecules of the sample may be tagged with sample indexes and/or molecular barcodes (referred to generally as “tags”). In some embodiments, adapters (such as the modified Y-shaped adapters disclosed herein) comprise at least one tag. In some embodiments, the at least one tag comprises a molecular barcode.

Tags can be molecules, such as nucleic acids, containing information that indicates a feature of the molecule with which the tag is associated. For example, DNA molecules can bear a sample tag or sample index (which distinguishes molecules in one sample from those in a different sample), a partition tag (which distinguishes molecules in one partition from those in a different partition) and/or a molecular tag/molecular barcode (which distinguishes different molecules from one another (in both unique and non-unique tagging scenarios)).

Tagging strategies can be divided into unique tagging and non-unique tagging strategies. In unique tagging, all or substantially all of the molecules in a sample bear a different tag, so that reads can be assigned to original molecules based on tag information alone. Tags used in such methods are sometimes referred to as “unique tags”. In non-unique tagging, different molecules in the same sample can bear the same tag, so that other information in addition to tag information is used to assign a sequence read to an original molecule. Such information may include start and stop coordinate, coordinate to which the molecule maps, start or stop coordinate alone, etc. Tags used in such methods are sometimes referred to as “non-unique tags”. Accordingly, it is not necessary to uniquely tag every molecule in a sample. It suffices to uniquely tag molecules falling within an identifiable class within a sample. Thus, molecules in different identifiable families can bear the same tag without loss of information about the identity of the tagged molecule.

In certain embodiments, a tag can comprise one or a combination of barcodes. As used herein, the term “barcode” refers to a nucleic acid molecule having a particular nucleotide sequence, or to the nucleotide sequence, itself, depending on context. A barcode can have, for example, between 10 and 100 nucleotides. A collection of barcodes can have degenerate sequences or can have sequences having a certain Hamming distance, as desired for the specific purpose. So, for example, a molecular barcode can be comprised of one barcode or a combination of two barcodes, each attached to different ends of a molecule. Additionally or alternatively, for different partitions and/or samples, different sets of molecular barcodes, molecular tags, or molecular indexes can be used such that the barcodes serve as a molecular tag through their individual sequences and also serve to identify the partition and/or sample to which they correspond based the set of which they are a member.

Tags can be used to label the individual polynucleotide population partitions so as to correlate the tag (or tags) with a specific partition. Alternatively, tags can be used in embodiments of the disclosure that do not employ a partitioning step. In some embodiments, a single tag can be used to label a specific partition. In some embodiments, multiple different tags can be used to label a specific partition. In embodiments employing multiple different tags to label a specific partition, the set of tags used to label one partition can be readily differentiated for the set of tags used to label other partitions. In some embodiments, the tags may have additional functions, for example the tags can be used to index sample sources or used as unique molecular identifiers (which can be used to improve the quality of sequencing data by differentiating sequencing errors from mutations, for example as in Kinde et al., Proc Nat'l Acad Sci USA 108: 9530-9535 (2011), Kou et al., PLoS ONE, 11: e0146638 (2016)) or used as non-unique molecule identifiers, for example as described in U.S. Pat. No. 9,598,731. Similarly, in some embodiments, the tags may have additional functions, for example the tags can be used to index sample sources or used as non-unique molecular identifiers (which can be used to improve the quality of sequencing data by differentiating sequencing errors from mutations).

Tags may be incorporated into or otherwise joined to adapters by chemical synthesis, ligation (e.g., as described above, e.g., by blunt-end ligation or sticky-end ligation), or overlap extension polymerase chain reaction (PCR), among other methods. Such adapters are ultimately joined to the sample DNA molecule. In other embodiments, one or more rounds of amplification cycles (e.g., PCR amplification) may be applied to introduce sample indexes to a nucleic acid molecule using conventional nucleic acid amplification methods. The amplifications may be conducted in one or more reaction mixtures (e.g., a plurality of microwells in an array). Molecular barcodes and/or sample indexes may be introduced simultaneously, or in any sequential order. In some embodiments, molecular barcodes and/or sample indexes are introduced prior to and/or after any conversion procedure. In the case of molecular barcodes and/or sample indexes being introduced through amplification processes, the conversion step will occur before the molecular barcodes and/or sample indexes are introduced. In some embodiments, molecular barcodes and/or sample indexes are introduced prior to and/or after sequence capturing steps, if present, are performed. In some embodiments, only the molecular barcodes are introduced prior to probe capturing and the sample indexes are introduced after sequence capturing steps are performed. In some embodiments, both the molecular barcodes and the sample indexes are introduced prior to performing probe-based capturing steps, if present. In some embodiments, the sample indexes are introduced after sequence capturing steps are performed, if present. In some embodiments, sample indexes are incorporated through overlap extension polymerase chain reaction (PCR).

In some embodiments, the tags may be located at one end or at both ends of the sample DNA molecule. In some embodiments, tags are predetermined or random or semi-random sequence oligonucleotides. In some embodiments, the tag(s) may together be less than about 500, 200, 100, 50, 20, 10, 9, 8, 7, 6, 5, 4, 3, 2, or 1 nucleotides in length. Typically tags are about 5 to 20 or 6 to 15 nucleotides in length. The tags may be linked to sample DNA molecules randomly or non-randomly.

In some embodiments, each sample or partition (discussed below) is uniquely tagged with a sample index or a combination of sample indexes. In some embodiments, each nucleic acid molecule of a sample or sub-sample is uniquely tagged with a molecular barcode or a combination of molecular barcodes. In other embodiments, a plurality of molecular barcodes may be used such that molecular barcodes are not necessarily unique to one another in the plurality (e.g., non-unique molecular barcodes). In these embodiments, molecular barcodes are generally attached (e.g., by ligation as part of an adapter) to individual molecules such that the combination of the molecular barcode and the sequence it may be attached to creates a unique sequence that may be individually tracked. Detection of non-unique molecular barcodes in combination with endogenous sequence information (e.g., the beginning (start) and/or end (stop) genomic location/position corresponding to the sequence of the original DNA molecule in the sample, start and stop genomic positions corresponding to the sequence of the original DNA molecule in the sample, the beginning (start) and/or end (stop) genomic location/position of the sequence read that is mapped to the reference sequence, start and stop genomic positions of the sequence read that is mapped to the reference sequence, sub-sequences of sequence reads at one or both ends, length of sequence reads, and/or length of the original DNA molecule in the sample) typically allows for the assignment of a unique identity to a particular molecule. In some embodiments, beginning region comprises the first 1, first 2, the first 5, the first 10, the first 15, the first 20, the first 25, the first 30 or at least the first 30 base positions at the 5′ end of the sequencing read that align to the reference sequence. In some embodiments, the end region comprises the last 1, last 2, the last 5, the last 10, the last 15, the last 20, the last 25, the last 30 or at least the last 30 base positions at the 3′ end of the sequencing read that align to the reference sequence. The length, or number of base pairs, of an individual sequence read are also optionally used to assign a unique identity to a given molecule. As described herein, fragments from a single strand of nucleic acid having been assigned a unique identity, may thereby permit subsequent identification of fragments from the parent strand, and/or a complementary strand.

In certain embodiments of non-unique tagging, the number of different tags used can be sufficient that there is a very high likelihood (e.g., at least 99%, at least 99.9%, at least 99.99% or at least 99.999% that all DNA molecules of a particular group bear a different tag. It is to be noted that when barcodes are used as tags, and when barcodes are attached, e.g., randomly, to both ends of a molecule, the combination of barcodes, together, can constitute a tag. This number, in term, is a function of the number of molecules falling into the calls. For example, the class may be all molecules mapping to the same start-stop position on a reference genome. The class may be all molecules mapping across a particular genetic locus, e.g., a particular base or a particular region (e.g., up to 100 bases or a gene or an exon of a gene).

In certain embodiments, the number of different tags used to uniquely identify a number of molecules, z, in a class can be between any of 2*z, 3*z, 4*z, 5*z, 6*z, 7*z, 8*z, 9*z, 10*z, 11*z, 12*z, 13*z, 14*z, 15*z, 16*z, 17*z, 18*z, 19*z, 20*z or 100*z (e.g., lower limit) and any of 100,000*z, 10,000*z, 1000*z or 100*z (e.g., upper limit). In some embodiments, molecular barcodes are introduced at an expected ratio of a set of identifiers (e.g., a combination of unique or non-unique molecular barcodes) to molecules in a sample. One example format uses from about 2 to about 1,000,000 different molecular barcode sequences, or from about 5 to about 150 different molecular barcode sequences, or from about 20 to about 50 different molecular barcode sequences, ligated to both ends of a target molecule. Alternatively, from about 25 to about 1,000,000 different molecular barcode sequences may be used. For example, 20-50×20-50 molecular barcode sequences (i.e., one of the 20-50 different molecular barcode sequences can be attached to each end of the target molecule) can be used. Such numbers of identifiers are typically sufficient for different molecules having the same start and stop points to have a high probability (e.g., at least 94%, 99.5%, 99.99%, or 99.999%) of receiving different combinations of identifiers. In some embodiments, about 80%, about 90%, about 95%, or about 99% of molecules have the same combinations of molecular barcodes. For example, in a sample of about 5 ng to 30 ng of cell free DNA, one expects around 3000 molecules to map to a particular nucleotide coordinate, and between about 3 and 10 molecules having any start coordinate to share the same stop coordinate. Accordingly, about 50 to about 50,000 different tags (e.g., between about 6 and 220 barcode combinations) can suffice to uniquely tag all such molecules. To uniquely tag all 3000 molecules mapping across a nucleotide coordinate, about 1 million to about 20 million different tags would be required.

In some embodiments, the assignment of unique or non-unique molecular barcodes in reactions is performed using methods and systems described in, for example, U.S. Patent Application Nos. 20010053519, 20030152490, and 20110160078, and U.S. Pat. Nos. 6,582,908, 7,537,898, 9,598,731, and 9,902,992, each of which is hereby incorporated by reference in its entirety. Alternatively, in some embodiments, different nucleic acid molecules of a sample may be identified using only endogenous sequence information (e.g., start and/or stop positions, sub-sequences of one or both ends of a sequence, and/or lengths). Tags can be linked to sample nucleic acids randomly or non-randomly.

In some embodiments, the tagged nucleic acids are sequenced after loading into a microwell plate. The microwell plate can have 96, 384, or 1536 microwells. In some cases, they are introduced at an expected ratio of unique tags to microwells. For example, the unique tags may be loaded so that more than about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags are loaded per genome sample. In some cases, the unique tags may be loaded so that less than about 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags are loaded per genome sample. In some cases, the average number of unique tags loaded per sample genome is less than, or greater than, about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags per genome sample.

In some embodiments a format uses 20-50 different tags (e.g., barcodes) ligated to both ends of target nucleic acids. For example, 35 different tags (e.g., barcodes) ligated to both ends of target molecules creating 35×35 permutations, which equals 1225 for 35 tags. Such numbers of tags are sufficient so that different molecules having the same start and stop points have a high probability (e.g., at least 94%, 99.5%, 99.99%, 99.999%) of receiving different combinations of tags. Other barcode combinations include any number between 10 and 500, e.g., about 15×15, about 35×35, about 75×75, about 100×100, about 250×250, about 500×500.

In some cases, unique tags may be predetermined or random or semi-random sequence oligonucleotides. In other cases, a plurality of barcodes may be used such that barcodes are not necessarily unique to one another in the plurality. In this example, barcodes may be ligated to individual molecules such that the combination of the barcode and the sequence it may be ligated to creates a unique sequence that may be individually tracked. As described herein, detection of non-unique barcodes in combination with sequence data of beginning (start) and end (stop) portions of sequence reads may allow assignment of a unique identity to a particular molecule. The length or number of base pairs, of an individual sequence read may also be used to assign a unique identity to such a molecule. As described herein, fragments from a single strand of nucleic acid having been assigned a unique identity, may thereby permit subsequent identification of fragments from the parent strand.

In some embodiments, the method includes adding one or more internal control DNAs and forward and reverse primers for amplifying the internal control DNAs. The internal control DNAs may be added before amplification using the primers that anneal upstream and downstream of the rearrangement breakpoints. The forward and reverse primers for amplifying the internal control DNAs may be included with, or added at the same time as, the primers that anneal upstream and downstream of the rearrangement breakpoints. The internal control DNAs may comprise or consist of sequences that do not occur in the genome of the subject, or that do not occur in the genome of the species of which the subject is a member (e.g., the human genome). The forward and/or reverse primers for amplifying the internal control DNAs may comprise sequences that are not complementary to any sequence in the genome of the subject, e.g., the human genome. The internal control DNAs may be used to ensure that the amplification process proceeded as designed. As such, the method may comprise detecting (e.g., sequencing) molecules amplified from and/or captured by the one or more internal control DNAs. The method can comprise comparing an amount of internal control DNAs (e.g., number of molecules or reads detected that correspond to an internal control DNA sequence) to a predetermined threshold, and either rejecting sequencing results if the predetermined threshold is not met or accepting sequencing results if the predetermined threshold is met. The predetermined threshold may be established, e.g., based on historical data or by testing the method on samples of DNA from test subjects, such as healthy volunteers. For example, amplification and detection of the one or more internal control DNAs provides confirmation that the amplification process proceeded properly, thus reducing the likelihood of a false negative.

### G. Conversion Procedures

In some embodiments, methods disclosed herein comprise a step of subjecting DNA (e.g., cell-free DNA or DNA from a sample comprising cells, such as a blood sample (e.g., a whole blood sample, a buffy coat sample, a leukapheresis sample, or a PBMC sample) and/or additional DNA) to a conversion procedure that affects a first nucleobase in the DNA differently from a second nucleobase in the DNA. In some embodiments, the first nucleobase is a modified or unmodified nucleobase, the second nucleobase is a modified or unmodified nucleobase different from the first nucleobase, and the first nucleobase and the second nucleobase have the same base pairing specificity. In some embodiments, the procedure chemically converts the first or second nucleobase such that the base pairing specificity of the converted nucleobase is altered. In some embodiments, if the first nucleobase is a modified or unmodified adenine, then the second nucleobase is a modified or unmodified adenine; if the first nucleobase is a modified or unmodified cytosine, then the second nucleobase is a modified or unmodified cytosine; if the first nucleobase is a modified or unmodified guanine, then the second nucleobase is a modified or unmodified guanine; and if the first nucleobase is a modified or unmodified thymine, then the second nucleobase is a modified or unmodified thymine (where modified and unmodified uracil are encompassed within modified thymine for the purpose of this step). In some embodiments, the conversion procedure comprises deamination of unmodified cytosines of the DNA to uracil. In some embodiments, the conversion procedure comprises contacting the DNA or a subsample thereof with a cytosine deaminase, such as an APOBEC enzyme, optionally wherein the APOBEC enzyme is APOBEC3A. In some embodiments, the conversion procedure comprises enzymatic protection of one or more modified nucleobases of the DNA, such as glucosylation of the 5-hydroxymethylcytosines of the DNA, optionally wherein the glucosylation comprises contacting the DNA with beta-glucosyltransferase.

In some embodiments, the first nucleobase is a modified or unmodified cytosine, then the second nucleobase is a modified or unmodified cytosine. For example, the first nucleobase may comprise unmodified cytosine (C) and the second nucleobase may comprise one or more of 5-methylcytosine (mC) and 5-hydroxymethylcytosine (hmC). Alternatively, the second nucleobase may comprise C and the first nucleobase may comprise one or more of mC and hmC. Other combinations are also possible, as indicated, e.g., in the Summary above and the following discussion, such as where one of the first and second nucleobases comprises mC and the other comprises hmC. In some embodiments, the first nucleobase comprises unmodified cytosine (C) and the second nucleobase comprises 5-methylcytosine (mC). In other embodiments, the first nucleobase comprises unmodified cytosine (C) and the second nucleobase comprises 5-hydroxymethylcytosine (hmC). In some embodiments, the procedure that affects a first nucleobase of the DNA differently from a second nucleobase of the DNA is methylation-sensitive conversion.

In some embodiments, the procedure that affects a first nucleobase in the DNA differently from a second nucleobase in the DNA comprises bisulfite conversion. Treatment with bisulfite converts unmodified cytosine and certain modified cytosines (e.g., 5-formyl cytosine (fC) or 5-carboxylcytosine (caC)) to uracil whereas other modified cytosines (e.g., 5-methylcytosine, 5-hydroxylmethylcystosine) are not converted. Thus, where bisulfite conversion is used, the first nucleobase comprises one or more of unmodified cytosine, 5-formyl cytosine, 5-carboxylcytosine, or other cytosine forms affected by bisulfite, and the second nucleobase may comprise one or more of mC and hmC, such as mC and optionally hmC. Sequencing of bisulfite-treated DNA identifies positions that are read as cytosine as being mC or hmC positions. Meanwhile, positions that are read as T are identified as being T or a bisulfite-susceptible form of C, such as unmodified cytosine, 5-formyl cytosine, or 5-carboxylcytosine. Performing bisulfite conversion, such as on a DNA sample as described herein, thus facilitates identifying positions containing mC or hmC using the sequence reads obtained from the exemplary sample. For an exemplary description of bisulfite conversion, see, e.g., Moss et al., Nat Commun. 2018; 9: 5068.

In some embodiments, the procedure that affects a first nucleobase in the DNA differently from a second nucleobase in the DNA comprises oxidative bisulfite (Ox-BS) conversion. This procedure first converts hmC to fC, which is bisulfite susceptible, followed by bisulfite conversion. Thus, when oxidative bisulfite conversion is used, the first nucleobase comprises one or more of unmodified cytosine, fC, caC, hmC, or other cytosine forms affected by bisulfite, and the second nucleobase comprises mC. Sequencing of Ox-BS converted DNA identifies positions that are read as cytosine as being mC positions. Meanwhile, positions that are read as T are identified as being T, hmC, or a bisulfite-susceptible form of C, such as unmodified cytosine, fC, or hmC. Performing Ox-BS conversion, such as on a DNA sample as described herein, thus facilitates identifying positions containing mC using the sequence reads obtained from the sample. For an exemplary description of oxidative bisulfite conversion, see, e.g., Booth et al., Science 2012; 336: 934-937.

In some embodiments, the procedure that affects a first nucleobase in the DNA differently from a second nucleobase in the DNA comprises Tet-assisted bisulfite (TAB) conversion. In TAB conversion, hmC is protected from conversion and mC is oxidized in advance of bisulfite treatment, so that positions originally occupied by mC are converted to U while positions originally occupied by hmC remain as a protected form of cytosine. For example, as described in Yu et al., Cell 2012; 149: 1368-80, β-glucosyl transferase can be used to protect hmC (forming 5-glucosylhydroxymethylcytosine (ghmC)), then a TET protein such as mTet1 can be used to convert mC to caC, and then bisulfite treatment can be used to convert C and caC to U while ghmC remains unaffected. Thus, when TAB conversion is used, the first nucleobase comprises one or more of unmodified cytosine, fC, caC, mC, or other cytosine forms affected by bisulfite, and the second nucleobase comprises hmC. Sequencing of TAB-converted DNA identifies positions that are read as cytosine as being hmC positions. Meanwhile, positions that are read as T are identified as being T, mC, or a bisulfite-susceptible form of C, such as unmodified cytosine, fC, or caC. Performing TAB conversion, such as on a DNA sample as described herein, thus facilitates identifying positions containing hmC using the sequence reads obtained from the sample.

In some embodiments, the procedure that affects a first nucleobase in the DNA differently from a second nucleobase in the DNA comprises APOBEC-coupled epigenetic (ACE) conversion. In ACE conversion, an AID/APOBEC family DNA deaminase enzyme such as APOBEC3A (A3A) is used to deaminate unmodified cytosine and mC without deaminating hmC, fC, or caC. Thus, when ACE conversion is used, the first nucleobase comprises unmodified C and/or mC (e.g., unmodified C and optionally mC), and the second nucleobase comprises hmC. Sequencing of ACE-converted DNA identifies positions that are read as cytosine as being hmC, fC, or caC positions. Meanwhile, positions that are read as T are identified as being T, unmodified C, or mC. Performing ACE conversion on a DNA sample as described herein thus facilitates distinguishing positions containing hmC from positions containing mC or unmodified C using the sequence reads obtained from the sample. For an exemplary description of ACE conversion, see, e.g., Schutsky et al., Nature Biotechnology 2018; 36: 1083-1090. In some embodiments, the procedure that affects a first nucleobase in the DNA differently from a second nucleobase in the DNA comprises enzymatic conversion of the first nucleobase, e.g., as in EM-Seq. See, e.g., Vaisvila R, et al. (2019) EM-seq: Detection of DNA methylation at single base resolution from picograms of DNA. bioRxiv; DOI: 10.1101/2019.12.20.884692, available at biorxiv.org/content/10.1101/2019.12.20.884692v1. For example, TET2 and T4-PGT can be used to convert 5mC and 5hmC into substrates that cannot be deaminated by a deaminase (e.g., APOBEC3A), and then a deaminase (e.g., APOBEC3A) can be used to deaminate unmodified cytosines converting them to uracils.

In some embodiments, the procedure that affects a first nucleobase in the DNA differently from a second nucleobase in the DNA comprises enzymatic conversion of the first nucleobase, e.g., as in SEM-seq. See, e.g., Vaisvila et al. (2023) Discovery of novel DNA cytosine deaminase activities enables a nondestructive single-enzyme methylation sequencing method for base resolution high-coverage methylome mapping of cell-free and ultra-low input DNA. bioRxiv; DOI: 10.1101/2023.06.29.547047, available at biorxiv.org/content/10.1101/2023.06.29.547047v1. SEM-Seq employs a non-specific, modification-sensitive double-stranded DNA deaminase (MsddA) or a modification-sensitive DNA deaminase A (MsddA)-like deaminase in a nondestructive single-enzyme 5-methylctyosine sequencing (SEM-seq) method that deaminates unmodified cytosines. Accordingly, SEM-seq does not require the TET2/T4-OGT protection and denaturing steps that are of use, e.g., in APOEC3A-based protocols. Additionally, MsddA does not deaminate 5-formylated cytosines (5fC) or 5-carboxylated cytosines (5caC). In SEM-seq, unmodified cytosines in the DNA are deaminated to uracil and is read as “T” during sequencing. Modified cytosines (e.g., 5mC) are not converted and are read as “C” during sequencing. Cytosines that are read as thymines are identified as unmodified (e.g., unmethylated) cytosines or as thymines in the DNA. Performing SEM-seq conversion thus facilitates identifying positions containing 5mC using the sequence reads obtained.

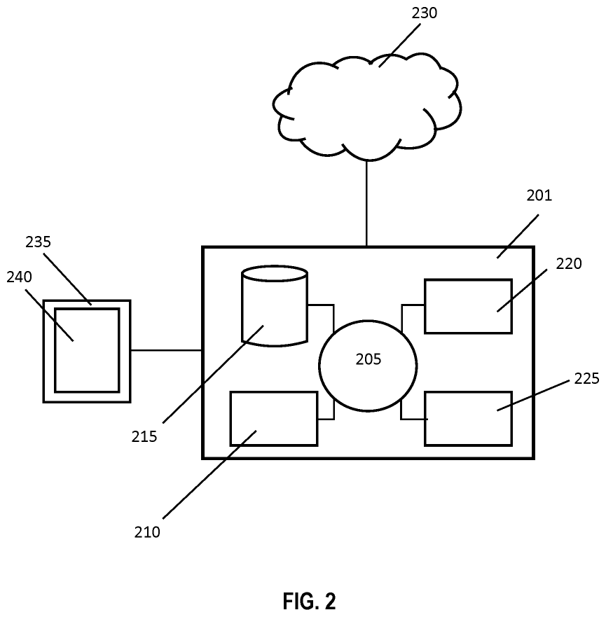

For an exemplary description of MsddA and MsddA-like deaminases, see, e.g., Vaisvila et al. Mol Cell. 2024 Mar. 7; 84(5):854-866.e7, which illustrates in FIG. 2A-C that MsddA-like deaminases have reduced activity on each of 5mC, 5hmC, and 5gmC relative to unmodified cytosine in dsDNA, e.g., a reduction of about 75%, 80%, or more on each of 5mC, 5hmC, and 5gmC relative to unmodified cytosine (e.g., using assay conditions as described in Vaisvila et al., such as analysis of deamination of C in E. coli or lambda dcm-DNA, deamination of 5mC in XP12 phage DNA, deamination of 5hmC in a C-hydroxymethylated adenovirus PCR fragment or fully C-hydroxymethylated T4147 phage DNA, and deamination of 5gmC in alpha-glucosyltransferase knockout (AGT-) T4 phage DNA. Deamination can be performed by contacting substrate DNA with deaminase and analyzed using NGS as follows: 50 ng of unmodified E. coli C2566 genomic DNA can be combined with the control DNAs (about 1 ng of Lambda, XP12, and T4147, and 0.1 ng of the 5hmC Adenovirus PCR fragment), sheared to about 300 bp and ligated to pyrrolo-dC adapters with 1 uL of in vitro synthesized deaminase (e.g., synthesized using the PURExpress In Vitro Protein Synthesis kit (NEB, Ipswich, MA) following manufacturer's recommendations with 100-400 ng of PCR fragment template DNA containing codon-optimized deaminase coding sequence and T7 promoter and terminator). Exemplary deamination reaction conditions are 50 mM Bis-Tris pH 6.0, 0.1% Triton X-100 for 1 hour at 37 degrees C. After the deamination reaction, 1 uL of Thermolabile Proteinase K (NEB, Ipswich, MA) can be added and incubated for 30 min at 37 degrees C and then the Proteinase K can be heat inactivated at 60 degrees C for 10 minutes. The deaminated product can then be used for library amplification using the NEBNext Q5U Master Mix (New England Biolabs, Ipswich, MA, USA) with 5 mM of NEBNext Unique Dual Index Primers. The resulting library can be purified using 1×NEBNext Sample Purification Beads according to the manufacturer's instructions and the purified library can be analyzed and quantified by an Agilent Bioanalyzer 2100 DNA Highsensitivity chip. The libraries can be sequenced using the Illumina NextSeq and NovaSeq platforms. Paired-end sequencing of 75 cycles (2×75 bp) can be performed for all the sequencing runs. Base calling and demultiplexing can be carried out with the standard Illumina pipeline.

In some embodiments, the procedure that affects a first nucleobase in the DNA differently from a second nucleobase in the DNA of the first subsample comprises separating DNA originally comprising the first nucleobase from DNA not originally comprising the first nucleobase. In some such embodiments, the first nucleobase is hmC. DNA originally comprising the first nucleobase may be separated from other DNA using a labeling procedure comprising biotinylating positions that originally comprised the first nucleobase. In some embodiments, the first nucleobase is first derivatized with an azide-containing moiety, such as a glucosyl-azide containing moiety. The azide-containing moiety then may serve as a reagent for attaching biotin, e.g., through Huisgen cycloaddition chemistry. Then, the DNA originally comprising the first nucleobase, now biotinylated, can be separated from DNA not originally comprising the first nucleobase using a biotin-binding agent, such as avidin, neutravidin (deglycosylated avidin with an isoelectric point of about 6.3), or streptavidin. An example of a procedure for separating DNA originally comprising the first nucleobase from DNA not originally comprising the first nucleobase is hmC-seal, which labels hmC to form 0-6-azide-glucosyl-5-hydroxymethylcytosine and then attaches a biotin moiety through Huisgen cycloaddition, followed by separation of the biotinylated DNA from other DNA using a biotin-binding agent. For an exemplary description of hmC-seal, see, e.g., Han et al., Mol. Cell 2016; 63: 711-719. This approach is useful for identifying fragments that include one or more hmC nucleobases.

In some embodiments, following such a separation, the method further comprises differentially tagging each of the DNA originally comprising the first nucleobase, the DNA not originally comprising the first nucleobase. The method may further comprise pooling the DNA originally comprising the first nucleobase and the DNA not originally comprising the first nucleobase following differential tagging. The DNA originally comprising the first nucleobase and the DNA not originally comprising the first nucleobase may then be used in downstream analyses. For example, the pooled DNA originally comprising the first nucleobase and the DNA not originally comprising the first nucleobase may be sequenced in the same sequencing cell (such as after being subjected to further treatments, such as those described herein) while retaining the ability to resolve whether a given read came from a molecule of DNA originally comprising the first nucleobase or DNA not originally comprising the first nucleobase using the differential tags.

In some embodiments, the first nucleobase is a modified or unmodified adenine, and the second nucleobase is a modified or unmodified adenine. In some embodiments, the modified adenine is N6-methyladenine (mA). In some embodiments, the modified adenine is one or more of N⁶-methyladenine (mA), N⁶-hydroxymethyladenine (hmA), or N⁶-formyladenine (fA).

Techniques comprising partitioning based on methylation status or methylated DNA immunoprecipitation (MeDIP) can be used to separate DNA containing modified bases such as mC, mA, caC (which may be generated by oxidation of mC or hmC with Tet2, e.g., before enzymatic conversion of unmodified C to U, e.g., using a deaminase such as APOBEC3A), or dihydrouracil from other DNA. See, e.g., Kumar et al., Frontiers Genet. 2018; 9: 640; Greer et al., Cell 2015; 161: 868-878. An antibody specific for mA is described in Sun et al., Bioessays 2015; 37:1155-62. Antibodies for various modified nucleobases, such as mC, caC, and forms of thymine/uracil including dihydrouracil or halogenated forms such as 5-bromouracil, are commercially available. Various modified bases can also be detected based on alterations in their base pairing specificity. For example, hypoxanthine is a modified form of adenine that can result from deamination and is read in sequencing as a G. See, e.g., U.S. Pat. No. 8,486,630; Brown, Genomes, 2^{nd} Ed., John Wiley & Sons, Inc., New York, N.Y., 2002, chapter 14, “Mutation, Repair, and Recombination.”

In some embodiments, the conversion procedure is an enzymatic conversion procedure which converts the base pairing specificity of modified nucleosides (e.g., DM-seq conversion comprising adding a protective group (such as a carboxymethyl group) to unmodified cytosines, and deaminating 5mC, such as using an APOBEC enzyme) or enzymatic conversion procedures which convert the base pairing specificity of unmodified nucleosides (such as SEM-seq).

In some cases, the conversion procedure used in the methods of the disclosure is one that changes the base pairing specificity of a modified nucleoside (e.g., methylated cytosine), but does not change the base pairing specificity of the corresponding unmodified nucleoside (e.g. cytosine) or does not change the base pairing specificity of any un-modified nucleoside (e.g. cytosine, adenosine, guanosine and thymidine (or uracil)). Advantages of methods that do not convert the base-pairing specificity of unmodified nucleosides include reduced loss of sequence complexity, higher sequencing efficiency and reduced alignment losses. Additionally, methods such as DM-seq may in some cases be preferred over methods such as bisulfite sequencing and EM-seq because they are less destructive (especially important for low yield samples such as cfDNA) and do not require denaturation, meaning that non-conversion errors are theoretically more likely to be random. In methods that require denaturation for conversion, failure to denature a DNA molecule will result in non-conversion of all bases in the DNA molecule. As biological changes in methylation are predominantly concerted to a localized region of interest, these non-random (localized) conversion can appear as false negatives (non-methylated regions). Random non-conversion methods can maximally affect a low percent of bases within a region, and thus the specificity of methylation change detection can be maximized (reduce false positives) by placing a threshold on % of bases within a region that are methylated/non-methylated. Hence, in some cases, a conversion procedure that does not involve denaturation is preferred.

In other cases, the conversion procedure used in the methods of the disclosure is one that changes the base pairing specificity of an unmodified nucleoside (e.g., cytosine), but does not change the base pairing specificity of the corresponding modified nucleoside (e.g., methylated cytosine).

The skilled person can select a suitable method according to their needs, including which nucleoside modifications are to be detected and/or identified.

In some embodiments, the conversion procedure converts modified nucleosides. In some embodiments, the conversion procedure which converts modified nucleosides comprises enzymatic conversion, such as DM-seq, for example, as described in WO2023/288222A1. In DM-seq, unmodified cytosines in the DNA are enzymatically protected from a subsequent deamination step wherein 5mC in 5mCpG is converted to T. The enzymatically protected unmodified (e.g., unmethylated) cytosines are not converted and are read as “C” during sequencing. Cytosines that are read as thymines (in a CpG context) are identified as methylated cytosines in the DNA.

Thus, when this type of conversion is used, the first nucleobase comprises unmodified (such as unmethylated) cytosine, and the second nucleobase comprises modified (such as methylated) cytosine. Sequencing of the converted DNA identifies positions that are read as cytosine as being unmodified C positions. Meanwhile, positions that are read as T are identified as being T or 5mC. Performing DM-seq conversion thus facilitates identifying positions containing 5mC using the sequence reads obtained.

Exemplary cytosine deaminases for use herein include APOBEC enzymes, for example, APOBEC3A. Generally, AID/APOBEC family DNA deaminase enzymes such as APOBEC3A (A3A) are used to deaminate (unprotected) unmodified cytosine and 5mC. For an exemplary description of APOBEC enzymes, see, e.g., Gajula et al., Nucleic Acids Res. 2014 September; 42(15):9964-75 and Schutsky et al., Nucleic Acids Res. 2017 Jul. 27; 45(13):7655-7665.

The enzymatic protection of unmodified cytosines in the DNA comprises addition of a protective group to the unmodified cytosines. Such protective groups can comprise an alkyl group, an alkyne group, a carboxyl group, a carboxyalkyl group, an amino group, a hydroxymethyl group, a glucosyl group, a glucosylhydroxymethyl group, an isopropyl group, or a dye. For example, DNA can be treated with a methyltransferase, such as a CpG-specific methyltransferase, which adds the protective group to unmodified cytosines. The term methyltransferase is used broadly herein to refer to enzymes capable of transferring a methyl or substituted methyl (e.g.,carboxymethyl) to a substrate (e.g., a cytosine in a nucleic acid). In some embodiments, the DNA is contacted with a CpG-specific DNA methyltransferase (MTase), such as a CpG-specific carboxymethyltransferase (CxMTase), and a substituted methyl donor, such as a carboxymethyl donor (e.g., carboxymethyl-S-adenosyl-L-methionine). See, e.g., WO2021/236778A2. In particular embodiments, the CxMTase can facilitate the addition of a protective carboxymethyl group to an unmethylated cytosine. In some embodiments, the unmethylated cytosine is unmodified cytosine. The carboxymethyl group can prevent deamination of the cytosine during a deamination step (such as a deamination step using an APOBEC enzyme, such as A3A). Substituted methyl or carboxymethyl donors useful in the disclosed methods include but are not limited to, S-adenosyl-L-methionine (SAM) analogs, optionally wherein the SAM analog is carboxy-S-adenosyl-L-methionine (CxSAM). SAM analogs are described, for example, in WO2022/197593A1. The MTase may be, for example, a CpG methyltransferase from Spiroplasma sp. strain MQ1 (M.SssI), DNA-methyltransferase 1 (DNMT1), DNA-methyltransferase 3 alpha (DNMT3A), DNA-methyltransferase 3 beta (DNMT3B), or DNA adenine methyltransferase (Dam). The CxMTase may be a CpG methyltransferase from Mycoplasma penetrans (M.MpeI).

In one embodiment, the methyltransferase enzyme is a variant of M.MpeI having an N374R substitution or an N374K substitution. The methyltransferase can further comprise one or more amino acid substitutions selected from a) substitution of one or both residues T300 and E305 with S, A, G, Q, D, or N; b) substitution of one or more residues A323, N306, and Y299 with a positively charged amino acid selected from K, R or H; and/or c) substitution of S323 with A, G, K, R or H, which may enhance the activity of the enzyme.

Optionally, the conversion procedure further includes enzymatic protection of 5hmCs, such as by glucosylation of the 5hmCs (e.g., using PGT), in the DNA prior to the deamination of unprotected modified cytosines. In this method, 5hmC can be protected from conversion, for example through glucosylation using β-glucosyl transferase (OGT), forming (forming 5-glucosylhydroxymethylcytosine) 5ghmC. This is described, for example, in Yu et al., Cell 2012; 149: 1368-80. Glucosylation of 5hmC can reduce or eliminate deamination of 5hmC by a deaminase such as APOBEC3A. Treatment with an MTase or CxMTase then adds a protecting group to unmodified (unmethylated) cytosines in the DNA. 5mC (but not protected, unmodified cytosine and not 5ghmC) is then deaminated (converted to T in the case of 5mC) by treatment with a deaminase, for example, an APOBEC enzyme (such as APOBEC3A). Sequencing of the converted DNA identifies positions that are read as cytosine as being either 5hmC or unmodified C positions. Meanwhile, positions that are read as T are identified as being T or 5mC. Performing DM-seq conversion with glucosylation of 5hmC on a sample as described herein thus facilitates distinguishing positions containing unmodified C or 5hmC on the one hand from positions containing 5mC using the sequence reads obtained.

Also provided herein are methods in which alternative base conversion schemes are used. For example, unmethylated cytosines can be left intact while methylated cytosines and hydroxymethylcytosines are converted to a base read as a thymine (e.g., uracil, thymine, or dihydrouracil).

In some embodiments, methylating a cytosine in at least one first complementary strand or second complementary strand comprises contacting the cytosine with a methyltransferase such as DNMT1 or DNMT5. In such embodiments, the step of oxidizing a 5-hydroxymethylated cytosine to 5-formylcytosine (such as by contacting the 5-hydroxymethyl cytosine in a first strand and a second strand with KRuO₄) can be optional.

In some embodiments, converting the modified cytosine in at least one first or second strand to a thymine or a base read as thymine comprises oxidizing a hydroxymethyl cytosine, e.g., the hydroxymethyl cytosine is oxidized to formylcytosine. In some embodiments, oxidizing the hydroxymethyl cytosine to formylcytosine comprises contacting the hydroxymethyl cytosine with a ruthenate, such as potassium ruthenate (KRuO₄).

In some embodiments, the modified cytosine is converted to thymine, uracil, or dihydrouracil.

In some embodiments, the method comprises converting a formylcytosine and/or a methylcytosine to carboxylcytosine as part of converting the modified cytosine in at least one first or second strand to a thymine or a base read as thymine. For example, converting the formylcytosine and/or the methylcytosine to carboxylcytosine can comprise contacting the formylcytosine and/or the methylcytosine with a TET enzyme, such as TET1, TET2, or TET3. In some embodiments, the method comprises reducing the carboxylcytosine as part of converting the modified cytosine in at least one first or second strand to a thymine or a base read as thymine, and/or the carboxylcytosine is reduced to dihydrouracil. In some embodiments, reducing the carboxylcytosine comprises contacting the carboxylcytosine with a borane or borohydride reducing agent.

In some embodiments, the borane or borohydride reducing agent comprises pyridine borane, 2-picoline borane, borane, tert-butylamine borane, ammonia borane, sodium borohydride, sodium cyanoborohydride (NaBH₃CN), lithium borohydride (LiBH₄), ethylenediamine borane, dimethylamine borane, sodium triacetoxyborohydride, morpholine borane, 4-methylmorpholine borane, trimethylamine borane, dicyclohexylamine borane, or a salt thereof. In other embodiments, the reducing agent comprises lithium aluminum hydride, sodium amalgam, amalgam, sulfur dioxide, dithionate, thiosulfate, iodide, hydrogen peroxide, hydrazine, diisobutylaluminum hydride, oxalic acid, carbon monoxide, cyanide, ascorbic acid, formic acid, dithiothreitol, beta-mercaptoethanol, or any combination thereof.

Various TET enzymes may be used in the disclosed methods as appropriate. In some embodiments, the one or more TET enzymes comprise TETv. TETv is described in U.S. Pat. No. 10,260,088 and its sequence is SEQ ID NO: 1 therein. In some embodiments, the one or more TET enzymes comprise TETcd. TETcd is described in U.S. Pat. No. 10,260,088 and its sequence is SEQ ID NO: 3 therein. In some embodiments, the one or more TET enzymes comprise TET1. In some embodiments, the one or more TET enzymes comprise TET2. TET2 may be expressed and used as a fragment comprising TET2 residues 1129-1480 joined to TET2 residues 1844-1936 by a linker as described, e.g., in U.S. Pat. No. 10,961,525. In some embodiments, the one or more TET enzymes comprise TET1 and TET2. In some embodiments, the one or more TET enzymes comprise a V1900 TET mutant, such as a V1900A, V1900C, V1900G, V19001, or V1900P TET mutant. In some embodiments, the one or more TET enzymes comprise a V1900 TET2 mutant, such as a V1900A, V1900C, V1900G, V19001, or V1900P TET2 mutant. It can be beneficial to use a TET enzyme that maximizes formation of 5-carboxylcytosine (5-caC) relative to less oxidized modified cytosines, particularly 5-formylcytosine, because 5-caC is not a substrate for enzymatic deamination, e.g., by APOBEC enzymes such as APOBEC3A. Maximizing formation of 5-caC thus reduces the risk of false calls in which a base is identified as unmethylated because it underwent deamination even though it was methylated (or hydroxymethylated) in the original sample. Accordingly, in some embodiments, the TET enzyme comprises a mutation that increases formation of 5-caC. Exemplary mutations are set forth above. “A mutation that increases formation of 5-caC” means that the TET enzyme having the mutation produces more 5-caC than a TET enzyme that lacks the mutation but is otherwise identical. 5-caC production can be measured as described, e.g., in Liu et al., Nat Chem Biol 13:181-187 (2017) (see Online Methods section, TET reactions in vitro subsection, “driving” conditions).

### H. Samples and Subjects

The disclosure relates to methods of performing a sequencing by synthesis reaction on a DNA sample, comprising a step of modified end-repair (such as end repair performed using a modified dCTP, such as d5mCTP) and/or the use of modified NGS adapters (e.g., modified Y-shaped adapters). In some cases, the DNA sample is obtained or has been obtained from a subject. In some embodiments, the DNA sample may comprise or consist of DNA from a biological sample obtained from a subject. The subject may be a human, a mammal, an animal, a primate, rodent (including mice and rats), or other common laboratory, domestic, companion, service or agricultural animal, for example a rabbit, dog, cat, horse, cow, sheep, goat or pig. Preferably, the DNA sample is from a human. The subject may in some cases have or be suspected of having a cancer, tumor or neoplasm. In other cases the subject may not have cancer or a detectable cancer symptom. The subject may have been treated with one or more cancer therapy, e.g., any one or more of chemotherapies, antibodies, vaccines or biologics. The subject may be in remission, e.g., from a tumor, cancer, or neoplasia (e.g., following treatment such as chemotherapy, surgical resection, radiation, or a combination thereof). The subject may or may not be diagnosed as being susceptible to cancer or any cancer-associated genetic mutations/disorders. In some embodiments, the sample is a DNA sample obtained from a tumor tissue biopsy. The cancer, tumor, or neoplasm may generally be of any type, for example a cancer tumor or neoplasm of the lung, colon, rectum (or colorectum), kidney, breast, prostate, or liver, or other type of cancer as described herein. In some embodiments, the sample is obtained from a subject in remission from a tumor, cancer, or neoplasia (e.g., following chemotherapy, surgical resection, radiation, or a combination thereof). In any of the foregoing embodiments, the pre-cancer, cancer, tumor, or neoplasia or suspected pre-cancer, cancer, tumor, or neoplasia may be of the bladder, head and neck, lung, colon, rectum, kidney, breast, prostate, skin, or liver. In some embodiments, the pre-cancer, cancer, tumor, or neoplasia or suspected pre-cancer, cancer, tumor, or neoplasia is of the lung. In some embodiments, the pre-cancer, cancer, tumor, or neoplasia or suspected pre-cancer, cancer, tumor, or neoplasia is of the colon or rectum. In some embodiments, the pre-cancer, cancer, tumor, or neoplasia or suspected pre-cancer, cancer, tumor, or neoplasia is of the breast. In some embodiments, the pre-cancer, cancer, tumor, or neoplasia or suspected pre-cancer, cancer, tumor, or neoplasia is of the prostate. In any of the foregoing embodiments, the subject may be a human subject. In some embodiments, the sample is obtained from a subject having a stage I cancer, stage II cancer, stage III cancer or stage IV cancer.

In some embodiments, the subject may have an infection, a transplant rejection, or other disease or disorder related to changes in the immune system. The subject may not have cancer or a detectable cancer symptom. The subject may have been treated with one or more cancer therapy, e.g., any one or more of chemotherapies, antibodies, vaccines or biologics. The subject may be in remission. The subject may or may not be diagnosed as being susceptible to cancer or any cancer-associated genetic mutations/disorders.

The biological sample can be any biological sample isolated from a subject. Biological samples can include body tissues, such as known or suspected solid tumors, whole blood, platelets, serum, plasma, stool, red blood cells, white blood cells or leucocytes, endothelial cells, tissue biopsies, cerebrospinal fluid synovial fluid, lymphatic fluid, ascites fluid, interstitial or extracellular fluid, the fluid in spaces between cells, including gingival crevicular fluid, bone marrow, pleural effusions, cerebrospinal fluid, saliva, mucous, sputum, semen, sweat, urine. In some embodiments, biological samples are body fluids, particularly blood and fractions thereof, or urine. A sample can be in the form originally isolated from a subject or can have been subjected to further processing to remove or add components, such as cells, or enrich for one component relative to another. A sample can be isolated or obtained from a subject and transported to a site of sample analysis. The sample may be preserved and shipped at a desirable temperature, e.g., room temperature, 4 °C, −20 °C, and/or −80 °C. A sample can be isolated or obtained from a subject at the site of the sample analysis. The subject can be a human, a mammal, an animal, a companion animal, a service animal, or a pet. The subject may have a cancer, precancer, infection, transplant rejection, or other disease or disorder related to changes in the immune system. The subject may not have cancer or a detectable cancer symptom. The subject may have been treated with one or more cancer therapy, e.g., any one or more of chemotherapies, antibodies, vaccines, or biologics. The subject may be in remission. The subject may or may not be diagnosed of being susceptible to cancer or any cancer-associated genetic mutations/disorders.

In some embodiments, DNA to be sequenced is isolated from a tissue sample, such as a tumor sample. In some embodiments, the DNA to be sequenced is isolated from cells of a blood sample, such as a buffy coat sample, a whole blood sample, a leukapheresis sample, or a PBMC sample. In any of the embodiments of this disclosure, the DNA isolated from any type of sample comprising cells, including but not limited to a blood sample (e.g., a buffy coat sample, a whole blood sample, a leukapheresis sample, or a PBMC sample) may be DNA isolated from the cells of that sample.

Various approaches are described herein. In any of these embodiments, DNA, such as DNA from a tumor sample, may also be analyzed (e.g., sequenced, captured, converted, and/or partitioned) to provide information, e.g., for quantifying cell contributions to the DNA (such as cancer cell contributions to the DNA and/or immune cell contributions to the DNA); for identifying other cell types contributing to the DNA; for detecting mutations in the DNA; and/or for detecting epigenetic differences, e.g., differential methylation, relative to healthy or normal DNA.

Some embodiments of the present disclosure comprise steps of isolating cell-free DNA from a sample, e.g., a blood sample. Other sample types that include cells (such as immune and/or cancer-derived cells), such as blood samples (e.g., a whole blood sample, a buffy coat sample, a leukapheresis sample, or a peripheral blood PBMC sample) may also be used in embodiments of the disclosed methods. The DNA may be isolated from the cells (such as PBMCs) of any such sample. Such DNA isolation methods can include, but are not limited to, organic extraction (such as using phenol-chloroform), nonorganic methods (such as salting out and proteinase K treatment), or an adsorption method (such as using silica- or cellulose-based technologies). For example, methods of isolating DNA from a sample using adsorption can include lysing the cells of the sample, separating soluble DNA in the sample from cell debris and other insoluble material, binding the DNA of interest to a purification matrix, washing proteins and other contaminants away from the matrix, and eluting the DNA from the matrix.

In one embodiment, the DNA sample comprises cell-free DNA. In another embodiment the DNA sample is a DNA sample from a formalin fixed paraffin embedded (FFPE) sample.

In some embodiments, a population of nucleic acids is obtained from a serum, plasma or blood sample from a subject suspected of having neoplasia, a tumor, precancer, or cancer or previously diagnosed with neoplasia, a tumor, precancer, or cancer. The population includes nucleic acids having varying levels of sequence variation, epigenetic variation, and/or post-replication or transcriptional modifications. Post-replication modifications include modifications of cytosine, particularly at the 5-position of the nucleobase, e.g., 5-methylcytosine, 5-hydroxymethylcytosine, 5-formylcytosine and 5-carboxylcytosine.

In some embodiments, the sample comprises plasma. The volume of plasma obtained can depend on the desired read depth for sequenced regions. Exemplary volumes are 0.4-40 mL, 5-20 mL, 10-20 mL, and 3-5 mL. For example, the volume can be 0.5 mL, 1 mL, 2 mL, 3 mL, 4 mL, 5 mL, 6 mL, 7 mL, 8 mL, 9 mL, 10 mL, 20 mL, 30 mL, or 40 mL. A volume of sampled plasma may be 5 to 20 mL. In some embodiments, the sample volume is 3-5 mL of plasma, such as 4 mL of plasma, per 10 mL whole blood.

In some embodiments, the sample comprises whole blood. Exemplary volumes of sampled whole blood are 0.4-40 mL, 5-20 mL, 10-20 mL, 1-6 mL, 1-3 mL, and 3-5 mL. For example, the volume can be 0.5 mL, 1 mL, 2 mL, 3 mL, 4 mL, 5 mL, 6 mL, 7 mL, 8 mL, 9 mL, 10 mL, 20 mL, 30 mL, or 40 mL. A volume of sampled whole blood may be 5 to 20 mL. In some embodiments, the sample volume is 1-5 mL of whole blood, such as 2.5 mL of whole blood.

In some embodiments, the sample comprises buffy coat separated from whole blood. Exemplary volumes of sampled buffy coat are 0.1-20 mL, 1-10 mL, 1-5 mL, 0.2-0.6 mL, and 0.3-0.5 mL. For example, the volume can be 0.1 mL, 0.2 mL, 0.3 mL, 0.4 mL, 0.5 mL, 0.6 mL, 0.7 mL, 0.8 mL, 0.9 mL, 1 mL, 2 mL, 3 mL, 4 mL, 5 mL 10 mL, or 20 mL. A volume of sampled buffy coat may be 1 to 10 mL. In some embodiments, the sample volume is 0.1-0.5 mL of buffy coat, such as 0.3 mL of buffy coat, per 10 mL whole blood.

In some embodiments, the sample comprises PBMCs separated from whole blood. Exemplary volumes of sampled PBMCs are 0.1-20 mL, 1-10 mL, 1-5 mL, 0.2-0.6 mL, and 0.3-0.5 mL. For example, the volume can be 0.1 mL, 0.2 mL, 0.3 mL, 0.4 mL, 0.5 mL, 0.6 mL, 0.7 mL, 0.8 mL, 0.9 mL, 1 mL, 2 mL, 3 mL, 4 mL, 5 mL 10 mL, or 20 mL. A volume of sampled PBMCs may be 1 to 10 mL. In some embodiments, the sample volume is 0.1-0.5 mL of PBMCs, such as 0.3 mL of PBMCs, per 10 mL whole blood.

In some embodiments, the sample comprises leukocytes separated from subject blood using leukapheresis. Exemplary volumes of sampled leukocytes from leukapheresis are 0.1-20 mL, 1-10 mL, 1-5 mL, 0.2-0.6 mL, and 0.3-0.5 mL. For example, the volume can be 0.1 mL, 0.2 mL, 0.3 mL, 0.4 mL, 0.5 mL, 0.6 mL, 0.7 mL, 0.8 mL, 0.9 mL, 1 mL, 2 mL, 3 mL, 4 mL, 5 mL, 10 mL, or 20 mL. A volume of sampled leukocytes from leukapheresis may be 1 to 10 mL. In some embodiments, the sample volume is 0.1-0.6 mL of leukocytes from leukapheresis, such as 0.4 mL of leukocytes, per 10 mL whole blood.

A sample can comprise various amount of nucleic acid that contains genome equivalents. For example, a sample of about 30 ng DNA can contain about 10,000 (104) haploid human genome equivalents. Similarly, a sample of about 100 ng of DNA can contain about 30,000 haploid human genome equivalents.

A sample can comprise nucleic acids from different sources, e.g., nucleic acids from cells and cell-free nucleic acids of the same subject, and nucleic acids from cells and cell-free nucleic acids of different subjects. In some embodiments, the nucleic acid may be DNA. A sample can comprise DNA carrying mutations. For example, a sample can comprise DNA carrying germline mutations and/or somatic mutations. Germline mutations refer to mutations existing in germline DNA of a subject. Somatic mutations refer to mutations originating in somatic cells of a subject, e.g., cancer cells. A sample can comprise DNA carrying cancer-associated mutations (e.g., cancer-associated somatic mutations). A sample can comprise an epigenetic variant, wherein the epigenetic variant associated with the presence of a genetic variant such as a cancer-associated mutation. In some embodiments, the sample comprises an epigenetic variant associated with the presence of a genetic variant, wherein the sample does not comprise the genetic variant.

The DNA sample may be or comprise cell free nucleic acids or cfDNA. The cfDNA may be obtained from a test subject, for example as described above. For example, the sample for analysis may be plasma or serum containing cell-free nucleic acids. “Cell-free DNA” “cfDNA molecules,” or “cfDNA”, for example, include DNA molecules that naturally occur in a subject in extracellular form (e.g., in blood, serum, plasma, or other bodily fluids such as lymph, cerebrospinal fluid, urine, or sputum). While the cfDNA originally existed in a cell or cells in a large complex biological organism, e.g., a mammal, it has undergone release from the cell(s) in vivo into a fluid found in the organism, and may be obtained by obtaining a sample of the fluid without the need to perform an in vitro cell lysis step. In other words, cell-free nucleic acids or cfDNA are nucleic acids or DNA not contained within or otherwise bound to a cell, or the nucleic acids or DNA remaining in a sample after removing intact cells. Cell-free nucleic acids include DNA, RNA, and hybrids thereof, including genomic DNA, mitochondrial DNA, siRNA, miRNA, circulating RNA (cRNA), tRNA, rRNA, small nucleolar RNA (snoRNA), Piwi-interacting RNA (piRNA), long non-coding RNA (long ncRNA), or fragments of any of these. Cell-free nucleic acids can be double-stranded, single-stranded, or a hybrid thereof. A cell-free nucleic acid can be released into bodily fluid through secretion or cell death processes, e.g., cellular necrosis and apoptosis. Some cell-free nucleic acids are released into bodily fluid from cancer cells e.g., circulating tumor DNA, (ctDNA). Others are released from healthy cells. In some embodiments, cfDNA is cell-free fetal DNA (cffDNA). In some embodiments, cell free nucleic acids are produced by tumor cells. In some embodiments, cell free nucleic acids are produced by a mixture of tumor cells and non-tumor cells.

Exemplary amounts of nucleic acids (e.g., DNA from a buffy coat sample or any other sample comprising cells, such as a blood sample (e.g., a whole blood sample, a leukapheresis sample, or a PBMC sample) in a sample before amplification range from about 1 fg to about 1 g, e.g., 1 pg to 200 ng, 1 ng to 100 ng, 10 ng to 1000 ng. For example, the amount can be up to about 600 ng, up to about 500 ng, up to about 400 ng, up to about 300 ng, up to about 200 ng, up to about 100 ng, up to about 50 ng, or up to about 20 ng of nucleic acid molecules. The amount can be at least 1 fg, at least 10 fg, at least 100 fg, at least 1 pg, at least 10 pg, at least 100 pg, at least 1 ng, at least 10 ng, at least 100 ng, at least 150 ng, or at least 200 ng of nucleic acid molecules. The amount can be up to 1 femtogram (fg), 10 fg, 100 fg, 1 picogram (pg), 10 pg, 100 pg, 1 ng, 10 ng, 100 ng, 150 ng, or 200 ng of nucleic acid molecules. The method can comprise obtaining 1 femtogram (fg) to 200 ng.

Cell-free nucleic acids have an exemplary size distribution of about 100-500 nucleotides, with molecules of 110 to about 230 nucleotides representing about 90% of molecules, with a mode of about 168 nucleotides and a second minor peak in a range between 240 to 440 nucleotides.

Nucleic acids can be isolated from cells, such as cells of bodily fluids. Cells can be lysed and cellular nucleic acids processed. Cell-free nucleic acids can be isolated from bodily fluids through a fractionation or partitioning step in which cell-free nucleic acids, as found in solution, are separated from intact cells and other non-soluble components of the bodily fluid. Partitioning may include techniques such as centrifugation or filtration. Alternatively, cells in bodily fluids can be lysed and cell-free and cellular nucleic acids processed together. Generally, after addition of buffers and wash steps, nucleic acids can be precipitated with an alcohol. Further clean up steps may be used such as silica based columns to remove contaminants or salts. Non-specific bulk carrier nucleic acids, DNA or protein for sequencing (e.g., bisulfite sequencing), hybridization, and/or ligation, may be added throughout the reaction to optimize certain aspects of the procedure such as yield.

After such processing, samples can include various forms of nucleic acid including double stranded DNA, single stranded DNA and single stranded RNA. In some embodiments, single stranded DNA and RNA can be converted to double stranded forms so they are included in subsequent processing and analysis steps.

The methods disclosed herein can be used for the analysis of DNA from formalin-fixed paraffin-embedded (FFPE) tissue samples. While the formalin fixation process adequately preserves the ultrastructure of the tissues, it results in various types of damage to the DNA within the tissues, such as nicks in the DNA. As explained elsewhere herein, these nicks can lead to synthesis of regions of the DNA molecule in the end repair process. The methods disclosed herein allow for these regions to be identified and the sequence data to be interpreted accordingly.

DNA molecules can be linked to adapters at either one end or both ends. Typically, double-stranded molecules are blunt ended by treatment with a polymerase with a 5′-3′ polymerase and a 3′-5′ exonuclease (or proof-reading function), in the presence of all four standard nucleotides. Klenow large fragment and T4 polymerase are examples of suitable polymerase. The blunt ended DNA molecules can be ligated with at least partially double stranded adapter (e.g., a Y-shaped or bell-shaped adapter). Alternatively, complementary nucleotides can be added to blunt ends of sample nucleic acids and adapters to facilitate ligation. Contemplated herein are both blunt end ligation and sticky end ligation. In blunt end ligation, both the nucleic acid molecules and the adapter tags have blunt ends. In sticky-end ligation, typically, the nucleic acid molecules bear an “A” overhang and the adapters bear a “T” overhang.

### I. Amplification

Sample nucleic acids flanked by adapters can be amplified by PCR and other amplification methods. Amplification is typically primed by primers that anneal or bind to primer binding sites in adapters flanking a DNA molecule to be amplified. Amplification methods can involve cycles of denaturation, annealing and extension, resulting from thermocycling or can be isothermal as in transcription-mediated amplification. Other amplification methods include the ligase chain reaction, strand displacement amplification, nucleic acid sequence-based amplification, and self-sustained sequence based replication.

In some embodiments, the present methods perform dsDNA ligations with T-tailed and C-tailed adapters, which result in amplification of at least 50, 60, 70 or 80% of double stranded nucleic acids before linking to adapters. The present methods increase the amount or number of amplified molecules relative to control methods performed with T-tailed adapters alone by at least 10, 15 or 20%.

### J. Analysis

In some embodiments, a method described herein comprises performing a sequencing by synthesis reaction on a converted DNA molecule with a sequencing by synthesis instrument. In embodiments, the converted DNA molecule comprises ligated adapters, a converted region comprising one or more nucleobases that have been converted by a conversion procedure, and a resistant region comprising one or more nucleobases that are resistant to the conversion procedure. In such embodiments, the sequencing by synthesis reaction comprises extending a sequencing primer that binds to the converted DNA molecule upstream of the converted region and the resistant region, calibrating one or more base calling metrics of the sequencing by synthesis instrument based at least in part on data from a region of the converted DNA molecule that is resistant to the conversion procedure, and calling at least a portion of nucleobases in the converted region using the one or more calibrated base calling metrics.

In some embodiments, a method described herein comprises identifying the presence of DNA produced by a tumor (or neoplastic cells, or cancer cells) or by precancer cells. In some embodiments, a method described herein comprises identifying the presence of DNA produced by cells (such as immune cells) that are not tumor cells, cancer cells, or precancer cells. In some embodiments, a method described herein comprises determining the levels of particular cell types (such as at least one cancer cell type and/or at least one immune cell type) from which DNA originated. Exemplary immune cell types may comprise naïve lymphocytes, activated lymphocytes, myeloid cells at different points of differentiation, and/or other types. In some such embodiments, determination of immune cell distribution facilitates detection or diagnosis or cancer or precancer, or determination of cancer prognosis or cancer treatment options (such as prediction of a clinical outcome for a therapy, such as a chemotherapy or an immunotherapy, in a subject). In some embodiments, the determination of levels of cell types (such as at least one cancer cell type and/or at least one immune cell type) facilitates determination of the likelihood that the subject from which the DNA was obtained has a disease or disorder, such as a cancer or precancer, and/or a disease or disorder related to the immune system, such as an infection, or transplant rejection.

For example, determining the ratios of different cell types may facilitate such detection or determination. In some embodiments, wherein the cell types are immune cell types, the ratio numerator is the number or relative number of neutrophils, monocytes, or both, and the ratio denominator is the number or relative number of T cells, B cells, NK cells, or all lymphocytes. In some embodiments, the ratio numerator is the number or relative number of neutrophils, and the ratio denominator is the number or relative number of T cells, B cells, NK cells, or all lymphocytes. In some embodiments, the ratio numerator is the number or relative number of monocytes, and the ratio denominator is the number or relative number of T cells, B cells, NK cells, or all lymphocytes. In some embodiments, the ratio numerator is the number or relative number of neutrophils and monocytes, and the ratio denominator is the number or relative number of T cells, B cells, NK cells, or all lymphocytes. In some embodiments, the ratio is a neutrophil to lymphocyte ratio. In some embodiments, the ratio is a NK cell to total lymphocytes ratio. In some embodiments, the ratio numerator is the number or relative number of NK cells and the ratio denominator is the number or relative number of total lymphocytes. In some embodiments, the ratio is an M1 macrophage to M2 macrophage ratio. In some embodiments, the ratio numerator is the number or relative number of M1 macrophages and the ratio denominator is the number or relative number of M2 macrophages. In some embodiments, the ratio is a monocyte to T cell ratio. In some embodiments, elevations in such ratios are associated with cancer. In other embodiments, reductions in such ratios are associated with cancer.

The present methods can be used to diagnose presence of conditions, particularly cancer or precancer, in a subject, to characterize conditions (e.g., staging cancer or determining heterogeneity of a cancer), monitor response to treatment of a condition, effect prognosis risk of developing a condition or subsequent course of a condition. The present disclosure can also be useful in determining the efficacy of a particular treatment option. Successful treatment options may increase the amount of copy number variation or rare mutations detected in subject's blood if the treatment is successful as more cancers may die and shed DNA. In other examples, this may not occur. In another example, perhaps certain treatment options may be correlated with genetic profiles of cancers over time. This correlation may be useful in selecting a therapy.

Additionally, if a cancer is observed to be in remission after treatment, the present methods can be used to monitor residual disease or recurrence of disease.

The types and number of cancers that may be detected may include blood cancers, brain cancers, lung cancers, skin cancers, nose cancers, throat cancers, liver cancers, bone cancers, lymphomas, pancreatic cancers, skin cancers, bowel cancers, rectal cancers, thyroid cancers, bladder cancers, kidney cancers, mouth cancers, stomach cancers, solid state tumors, heterogeneous tumors, homogenous tumors and the like. Type and/or stage of cancer can be detected from genetic variations including mutations, rare mutations, indels, copy number variations, transversions, translocations, recombination, inversion, deletions, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, and abnormal changes in nucleic acid 5-methylcytosine.

Genetic data can also be used for characterizing a specific form of cancer. Cancers are often heterogeneous in both composition and staging. Genetic profile data may allow characterization of specific sub-types of cancer that may be important in the diagnosis or treatment of that specific sub-type. This information may also provide a subject or practitioner clues regarding the prognosis of a specific type of cancer and allow either a subject or practitioner to adapt treatment options in accord with the progress of the disease. Some cancers can progress to become more aggressive and genetically unstable. Other cancers may remain benign, inactive or dormant. The system and methods of this disclosure may be useful in determining disease progression.

Further, the methods of the disclosure may be used to characterize the heterogeneity of an abnormal condition in a subject. Such methods can include, e.g., generating a genetic profile of extracellular polynucleotides derived from the subject, wherein the genetic profile comprises a plurality of data resulting from copy number variation and rare mutation analyses. In some embodiments, an abnormal condition is cancer or precancer. In some embodiments, the abnormal condition may be one resulting in a heterogeneous genomic population. In the example of cancer, some tumors are known to comprise tumor cells in different stages of the cancer. In other examples, heterogeneity may comprise multiple foci of disease. Again, in the example of cancer, there may be multiple tumor foci, perhaps where one or more foci are the result of metastases that have spread from a primary site.

The present methods can be used to generate a profile, fingerprint, or set of data that is a summation of genetic information derived from different cells in a heterogeneous disease. Such a set of data may comprise copy number variation, epigenetic variation, or other mutation analyses alone or in combination.

The present methods can be used to diagnose, prognose, monitor or observe cancers, or other diseases. In some embodiments, the methods herein do not involve the diagnosing, prognosing or monitoring a fetus and as such are not directed to non-invasive prenatal testing. In other embodiments, these methodologies may be employed in a pregnant subject to diagnose, prognose, monitor or observe cancers or other diseases in an unborn subject whose DNA and other polynucleotides may co-circulate with maternal molecules.

In general, after sequencing, analysis of reads can be performed on a partition-by-partition level, as well as a whole DNA population level. Tags can be used to sort reads from different partitions. Analysis can include in silico analysis to determine genetic and epigenetic variation (one or more of methylation, chromatin structure, etc.) using sequence information, genomic coordinates length, coverage, and/or copy number. In some embodiments, higher coverage can correlate with higher nucleosome occupancy in genomic region while lower coverage can correlate with lower nucleosome occupancy or a nucleosome depleted region (NDR).

An exemplary method for performing a sequencing by synthesis reaction on a converted DNA molecule comprises the following steps:
1. Preparing an extracted DNA sample (e.g., cell-free DNA or DNA isolated from a sample comprising cells, such as a blood sample (e.g., a whole blood sample, a buffy coat sample, a leukapheresis sample, or a PBMC sample) and/or additional DNA) by ligating modified Y-shaped adapters (as disclosed herein, such as shown in FIG. 3, right side) comprising molecular tags to the DNA. The adapters are ligated to the DNA such that read 1 begins from the 3′ end of an adapter-ligated DNA molecule. In some embodiments, the adapter comprises modified bases (e.g., modified cytosines) such that the nucleobases of the adapter are resistant to a conversion procedure (e.g., resistant to deamination). In some embodiments, such as embodiments wherein DNA isolated from a sample comprising cells is used, DNA is fragmented prior to the ligating, e.g., by sonication or restriction digestion.
2. Subjecting the DNA molecules to an epigenetic base conversion procedure (e.g., a procedure described herein, such as cytosine deamination by DM-seq, EM-seq, or bisulfite).
3. Performing a sequencing by synthesis reaction on the converted DNA molecules using a sequencing by synthesis instrument (e.g., an NGS instrument).
4. Calibrating one or more base calling metrics of the sequencing by synthesis instrument based at least in part on data from a resistant region of the DNA molecule (such as a resistant region of the Y-shaped adapter), thereby providing one or more calibrated base calling metrics.
5. Calling at least a portion of nucleobases in a converted region of the DNA molecule using the one or more calibrated base calling metrics.

Another exemplary method for performing a sequencing by synthesis reaction on a converted DNA molecule comprises the following steps:
1. Preparing an extracted DNA sample (e.g., cell-free DNA or DNA isolated from a sample comprising cells, such as a blood sample (e.g., a whole blood sample, a buffy coat sample, a leukapheresis sample, or a PBMC sample) and/or additional DNA) by performing end repair using a modified dNTP mix comprising modified cytosines, such as d5mCTP, rather than dCTP, and optionally an A-tailing reaction. The modified cytosines of the dNTP mix are incorporated into the DNA molecule during end repair and are resistant to a conversion procedure. In some embodiments, such as embodiments wherein DNA isolated from a sample comprising cells is used, DNA is fragmented prior to end repair and A-tailing, e.g., by sonication or restriction digestion. The DNA molecules may further comprise ligated adapters.
2. Subjecting the adapted DNA molecules to an epigenetic base conversion procedure (e.g., a procedure described herein, such as cytosine deamination by DM-seq, EM-seq, or bisulfite).
3. Performing a sequencing by synthesis reaction on the converted DNA molecules using a sequencing by synthesis instrument (e.g., an NGS instrument). The sequencing by synthesis reaction comprises extending a sequencing primer that binds to the converted DNA molecule upstream of the converted region (comprising one or more nucleobases that have been converted by the conversion procedure) and the resistant region (comprising one or more nucleobases that are resistant to the conversion procedure, e.g., modified cytosines incorporated during end repair).
4. Calibrating one or more base calling metrics of the sequencing by synthesis instrument based at least in part on data from a resistant region of the DNA molecule (such as the resistant region generated during end repair), thereby providing one or more calibrated base calling metrics.
5. Calling at least a portion of nucleobases in a converted region of the DNA molecule using the one or more calibrated base calling metrics.

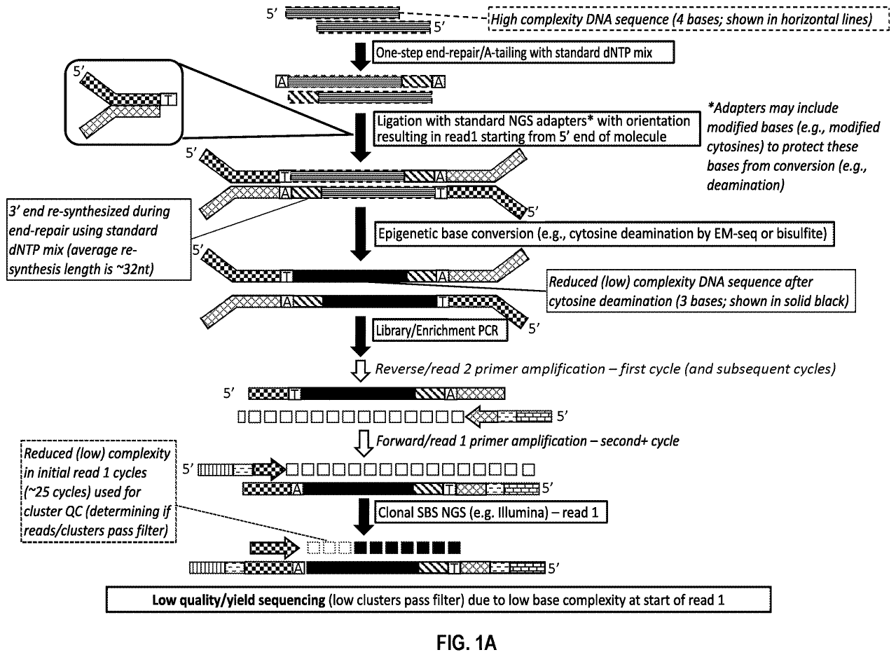

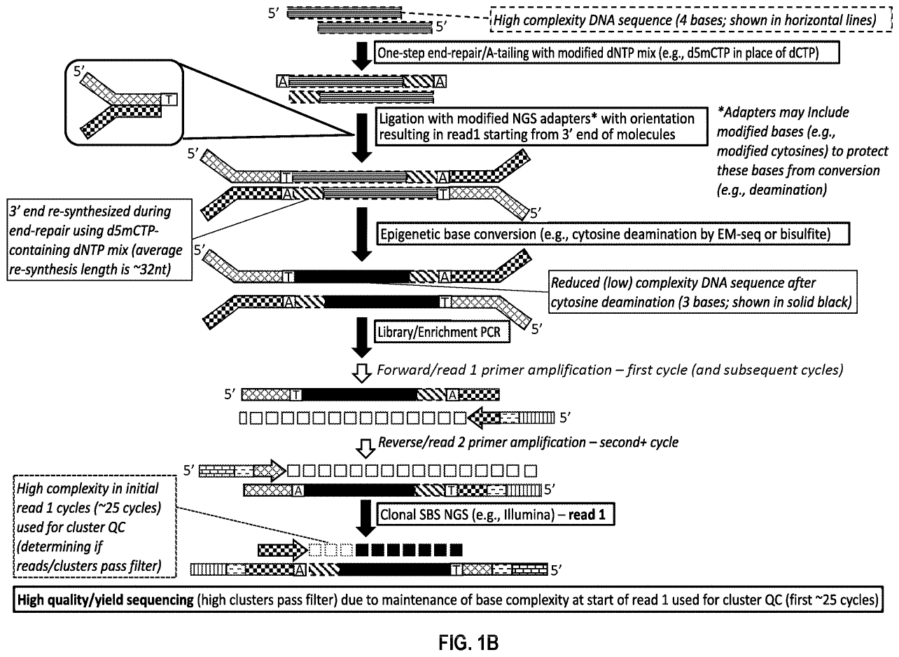

Another exemplary method for performing a sequencing by synthesis reaction on a converted DNA molecule comprises the following steps, and is shown in FIG. 1B (an exemplary standard workflow is illustrated in FIG. 1A for comparison purposes):
1. Preparing an extracted DNA sample (e.g., cell-free DNA or DNA isolated from a sample comprising cells, such as a blood sample (e.g., a whole blood sample, a buffy coat sample, a leukapheresis sample, or a PBMC sample) and/or additional DNA) by performing end repair using a modified dNTP mix comprising modified cytosines, such as d5mCTP, rather than dCTP, and optionally an A-tailing reaction. The modified cytosines of the dNTP mix are incorporated into the DNA molecule during end repair and are resistant to a conversion procedure. In some embodiments, such as embodiments wherein DNA isolated from a sample comprising cells is used, DNA is fragmented prior to end repair and optional A-tailing, e.g., by sonication or restriction digestion.
2. Ligating modified Y-shaped adapters (as disclosed herein, such as shown in FIG. 3, right side) comprising molecular tags to the DNA. The adapters are ligated to the DNA such that read 1 begins from the 3′ end of an adapter-ligated DNA molecule. In some embodiments, the adapter comprises modified bases (e.g., modified cytosines) such that the nucleobases of the adapter are resistant to a conversion procedure (e.g., resistant to deamination).
3. Subjecting the adapted DNA molecules to an epigenetic base conversion procedure (e.g., a procedure described herein, such as cytosine deamination by DM-seq, EM-seq, or bisulfite).
4. Performing a sequencing by synthesis reaction on the converted DNA molecules using a sequencing by synthesis instrument (e.g., an NGS instrument).
5. Calibrating one or more base calling metrics of the sequencing by synthesis instrument based at least in part on data from a resistant region of the DNA molecule (such as the resistant region generated during end repair and/or the resistant region of the Y-shaped adapter), thereby providing one or more calibrated base calling metrics.
6. Calling at least a portion of nucleobases in a converted region of the DNA molecule using the one or more calibrated base calling metrics.

In some embodiments of methods described herein, molecular tags (such as molecular barcodes) comprise or consist of nucleotides that are not altered by a procedure that affects a first nucleobase in the DNA differently from a second nucleobase in the DNA, such as any of those described herein (e.g., mC along with A, T, and G where the procedure is bisulfite conversion or any other conversion that does not affect mC; hmC along with A, T, and G where the procedure is a conversion that does not affect hmC; etc.). In some embodiments of methods described herein, the molecular tags do not comprise nucleotides that are altered by a procedure that affects a first nucleobase in the DNA differently from a second nucleobase in the DNA, such as any of those described herein (e.g., the tags do not comprise unmodified C where the procedure is bisulfite conversion or any other conversion that affects C; the tags do not comprise mC where the procedure is a conversion that affects mC; the tags do not comprise hmC where the procedure is a conversion that affects hmC; etc.).

### K. Partitioning

In some instances, a nucleic acid sample, such as a heterogeneous nucleic acid sample, is partitioned into two or more partitions (e.g., sub-samples). In some embodiments, each partition is differentially tagged. Tagged partitions can then be pooled together for collective sample prep and/or sequencing. The partitioning-tagging-pooling steps can occur more than once, with each round of partitioning occurring based on one or more different characteristics, and tagged using differential tags that are distinguished from other partitions and partitioning means.

In some embodiments of the disclosed methods, a partitioning step occurs prior to a step of sequencing by synthesis and prior to a step of performing a conversion procedure on a DNA molecule (such as on an adapted DNA molecule). In some embodiments of the disclosed methods, a partitioning step occurs prior to a step of sequencing by synthesis and after a step of performing a conversion procedure on a DNA molecule (such as on an adapted DNA molecule). In some embodiments, a partitioning step occurs prior to a step of sequencing by synthesis and prior to a step of tagging a DNA molecule. In some embodiments, a partitioning step occurs prior to a step of sequencing by synthesis and after a step of tagging a DNA molecule. In some embodiments, partitioning can be performed before a step of ligating adapters (such as Y-shaped adapters as disclosed herein) to DNA molecules, e.g., to facilitate including partition tags in the adapters. In other embodiments, partitioning can be performed after a step of ligating adapters (such as Y-shaped adapters as disclosed herein) to DNA molecules. In some embodiments, a partitioning step occurs prior to a step of pooling DNA from samples or subsamples as disclosed herein. In some embodiments, a partitioning step occurs prior to a step of subjecting the DNA molecules to a capture step as disclosed herein. In some embodiments, a partitioning step occurs after a step of subjecting the DNA molecules to a capture step as disclosed herein.

Examples of characteristics that can be used for partitioning include sequence length, methylation level, nucleosome binding, sequence mismatch, immunoprecipitation, and/or proteins that bind to DNA. Resulting partitions can include one or more of the following nucleic acid forms: single-stranded DNA (ssDNA), double-stranded DNA (dsDNA), shorter DNA fragments and longer DNA fragments. In some embodiments, partitioning based on a cytosine modification (e.g., cytosine methylation) or methylation generally is performed and is optionally combined with at least one additional partitioning step, which may be based on any of the foregoing characteristics or forms of DNA. In some embodiments, a heterogeneous population of nucleic acids is partitioned into nucleic acids with one or more base modifications and without the one or more base modifications. Examples of base modifications are described elsewhere herein. Alternatively or additionally, a heterogeneous population of nucleic acids can be partitioned into nucleic acid molecules associated with nucleosomes and nucleic acid molecules devoid of nucleosomes. Alternatively or additionally, a heterogeneous population of nucleic acids may be partitioned into single-stranded DNA (ssDNA) and double-stranded DNA (dsDNA). Alternatively, or additionally, a heterogeneous population of nucleic acids may be partitioned based on nucleic acid length (e.g., molecules of up to 160 bp and molecules having a length of greater than 160 bp).

In some cases, different procedures are applied to different partitions to determine different characteristics of the initial sample. For example, the DNA of at least one partition can be subjected to an end repair procedure according to the methods of the disclosure described herein. In some embodiments at least one partition is not subjected to the end repair procedure according to the methods of the disclosure described herein. In cases where a sequencing by synthesis procedure is performed on a converted molecule, corresponding sequences from a converted partition and non-converted partition can be compared to identify single nucleotides that have undergone conversion and therefore identify corresponding modified nucleosides in the initial sample.

In some embodiments, partition tagging comprises tagging molecules in each partition with a partition tag. After re-combining partitions (e.g., to reduce the number of sequencing runs needed and avoid unnecessary cost) and sequencing molecules, the partition tags identify the source partition. In another embodiment, different partitions are tagged with different sets of molecular tags, e.g., comprised of a pair of barcodes. In this way, each molecular barcode indicates the source partition as well as being useful to distinguish molecules within a partition. For example, a first set of 35 barcodes can be used to tag molecules in a first partition, while a second set of 35 barcodes can be used tag molecules in a second partition.

In some embodiments, after partitioning and tagging with partition tags, the molecules may be pooled for sequencing in a single run. In some embodiments, a sample tag is added to the molecules, e.g., in a step subsequent to addition of partition tags and pooling. Sample tags can facilitate pooling material generated from multiple samples for sequencing in a single sequencing run.

Alternatively, in some embodiments, partition tags may be correlated to the sample as well as the partition. As a simple example, a first tag can indicate a first partition of a first sample; a second tag can indicate a second partition of the first sample; a third tag can indicate a first partition of a second sample; and a fourth tag can indicate a second partition of the second sample.

While tags may be attached to molecules already partitioned based on one or more characteristics, the final tagged molecules in the library may no longer possess that characteristic. For example, while single stranded DNA molecules may be partitioned and tagged, the final tagged molecules in the library are likely to be double stranded. Similarly, while DNA may be subject to partition based on different levels of methylation, in the final library, tagged molecules derived from these molecules may be unmethylated. Accordingly, the tag attached to a molecule in the library can indicate the characteristic of the “parent molecule” from which the ultimate tagged molecule is derived, not necessarily to characteristic of the tagged molecule, itself.

As an example, barcodes 1, 2, 3, 4, etc. are used to tag and label molecules in the first partition; barcodes A, B, C, D, etc. are used to tag and label molecules in the second partition; and barcodes a, b, c, d, etc. are used to tag and label molecules in the third partition. Differentially tagged partitions can be pooled prior to sequencing. Differentially tagged partitions can be separately sequenced or sequenced together concurrently, e.g., in the same flow cell of an Illumina sequencer.

After sequencing, analysis of reads can be performed on a partition-by-partition level, as well as a whole DNA population level. Tags are used to sort reads from different partitions. Analysis can include in silico analysis to determine genetic and epigenetic variation (one or more of methylation, chromatin structure, etc.) using sequence information, genomic coordinates length, coverage, and/or copy number. In some embodiments, higher coverage can correlate with higher nucleosome occupancy in genomic region while lower coverage can correlate with lower nucleosome occupancy or a nucleosome depleted region (NDR).

Disclosed methods herein comprise performing a sequencing by synthesis reaction on a converted DNA molecule. In some embodiments described herein, the disclosed methods can comprise partitioning DNA. In such methods, different forms of DNA (e.g., hypermethylated and hypomethylated DNA) can be physically partitioned based on one or more characteristics of the DNA. This approach can be used to determine, for example, whether certain sequences are hypermethylated or hypomethylated. In some embodiments, a first subsample or aliquot of a sample is subjected to steps for making capture probes as described elsewhere herein and a second subsample or aliquot of a sample is subjected to partitioning. In some embodiments, a sample or subsample or aliquot thereof is subjected to partitioning and differential tagging, followed by a capture step using capture probes for rearranged sequences and optionally additional capture probes, e.g., for sequence-variable and/or epigenetic target regions.

Methylation profiling can involve determining methylation patterns across different regions of the genome. For example, after partitioning molecules based on extent of methylation (e.g., relative number of methylated nucleobases per molecule) and sequencing, the sequences of molecules in the different partitions can be mapped to a reference genome. This can show regions of the genome that, compared with other regions, are more highly methylated or are less highly methylated. In this way, genomic regions, in contrast to individual molecules, may differ in their extent of methylation.

Partitioning nucleic acid molecules in a sample can increase a rare signal, e.g., by enriching rare nucleic acid molecules that are more prevalent in one partition of the sample. For example, a genetic variation present in hypermethylated DNA but less (or not) present in hypomethylated DNA can be more easily detected by partitioning a sample into hypermethylated and hypomethylated nucleic acid molecules. By analyzing multiple partitions of a sample, a multi-dimensional analysis of a single molecule can be performed and hence, greater sensitivity can be achieved. Partitioning may include physically partitioning nucleic acid molecules into partitions or subsamples based on the presence or absence of one or more methylated nucleobases. A sample may be partitioned into partitions or subsamples based on a characteristic that is indicative of differential gene expression or a disease state. A sample may be partitioned based on a characteristic, or combination thereof that provides a difference in signal between a normal and diseased state during analysis of nucleic acids, e.g., cell free DNA (cfDNA), non-cfDNA, tumor DNA, circulating tumor DNA (ctDNA) and cell free nucleic acids (cfNA).

In some embodiments, hypermethylation and/or hypomethylation variable epigenetic target regions are analyzed to determine whether they show differential methylation characteristic of tumor cells or cells of a type that does not normally contribute to the DNA sample being analyzed (such as cfDNA), and/or particular cell types, such as immune cell types.

In some instances, heterogeneous DNA in a sample is partitioned into two or more partitions (e.g., at least 3, 4, 5, 6 or 7 partitions). In some embodiments, each partition is differentially tagged. Tagged partitions can then be pooled together for collective sample prep and/or sequencing. The partitioning-tagging-pooling steps can occur more than once, with each round of partitioning occurring based on a different characteristic (examples provided herein), and tagged using differential tags that are distinguished from other partitions and partitioning means. In other instances, the differentially tagged partitions are separately sequenced.

The agents used to partition populations of nucleic acids within a sample can be affinity agents, such as antibodies with the desired specificity, natural binding partners or variants thereof (Bock et al., Nat Biotech 28: 1106-1114 (2010); Song et al., Nat Biotech 29: 68-72 (2011)), or artificial peptides selected e.g., by phage display to have specificity to a given target. In some embodiments, the agent used in the partitioning is an agent that recognizes a modified nucleobase. In some embodiments, the modified nucleobase recognized by the agent is a modified cytosine, such as a methylcytosine (e.g., 5-methylcytosine). In some embodiments, the modified nucleobase recognized by the agent is a product of a procedure that affects the first nucleobase in the DNA differently from the second nucleobase in the DNA of the sample. In some embodiments, the modified nucleobase may be a “converted nucleobase,” meaning that its base pairing specificity was changed by a procedure. For example, certain procedures convert unmethylated or unmodified cytosine to dihydrouracil, or more generally, at least one modified or unmodified form of cytosine undergoes deamination, resulting in uracil (considered a modified nucleobase in the context of DNA) or a further modified form of uracil. Examples of partitioning agents include antibodies, such as antibodies that recognize a modified nucleobase, which may be a modified cytosine, such as a methylcytosine (e.g., 5-methylcytosine). In some embodiments, the partitioning agent is an antibody that recognizes a modified cytosine other than 5-methylcytosine, such as 5-carboxylcytosine (5caC). Alternative partitioning agents include methyl binding domain (MBDs) and methyl binding proteins (MBPs) as described herein, including proteins such as MeCP2.

Additional, non-limiting examples of partitioning agents are histone binding proteins which can separate nucleic acids bound to histones from free or unbound nucleic acids. Examples of histone binding proteins that can be used in the methods disclosed herein include RBBP4, RbAp48 and SANT domain peptides.

In some embodiments, partitioning can comprise both binary partitioning and partitioning based on degree/level of modifications. For example, methylated fragments can be partitioned by methylated DNA immunoprecipitation (MeDIP), or all methylated fragments can be partitioned from unmethylated fragments using methyl binding domain proteins (e.g., MethylMinder Methylated DNA Enrichment Kit (ThermoFisher Scientific). Subsequently, additional partitioning may involve eluting fragments having different levels of methylation by adjusting the salt concentration in a solution with the methyl binding domain and bound fragments. As salt concentration increases, fragments having greater methylation levels are eluted.

Analyzing DNA may comprise detecting or quantifying DNA of interest. Analyzing DNA can comprise detecting genetic variants and/or epigenetic features (e.g., DNA methylation and/or DNA fragmentation).

In some embodiments, methylation levels can be determined using partitioning, modification-sensitive conversion such as bisulfite conversion, direct detection during sequencing, methylation-sensitive restriction enzyme digestion, methylation-dependent restriction enzyme digestion, or any other suitable approach. For example, different forms of DNA (e.g., hypermethylated and hypomethylated DNA) can be physically partitioned based on one or more characteristics of the DNA. For example, a methylated DNA binding protein (e.g., an MBD such as MBD2, MBD4, or MeCP2) or an antibody specific for 5-methylcytosine (as in MeDIP) can be used to partition the DNA. This approach can be used to determine, for example, whether certain sequences are hypermethylated or hypomethylated. In some embodiments, DNA fragmentation pattern can be determined based on endpoints and/or centerpoints of DNA molecules, such as cfDNA molecules.

In some instances, the final partitions are enriched in nucleic acids having different extents of modifications (overrepresentative or underrepresentative of modifications). Overrepresentation and underrepresentation can be defined by the number of modifications born by a nucleic acid relative to the median number of modifications per strand in a population. For example, if the median number of 5-methylcytosine residues in nucleic acid in a sample is 2, a nucleic acid including more than two 5-methylcytosine residues is overrepresented in this modification and a nucleic acid with 1 or zero 5-methylcytosine residues is underrepresented. The effect of the affinity separation is to enrich for nucleic acids overrepresented in a modification in a bound phase and for nucleic acids underrepresented in a modification in an unbound phase (i.e., in solution). The nucleic acids in the bound phase can be eluted before subsequent processing.

When using MeDIP or MethylMiner® Methylated DNA Enrichment Kit (ThermoFisher Scientific) various levels of methylation can be partitioned using sequential elutions. For example, a hypomethylated partition (no methylation) can be separated from a methylated partition by contacting the nucleic acid population with the MBD from the kit, which is attached to magnetic beads. The beads are used to separate out the methylated nucleic acids from the non-methylated nucleic acids. Subsequently, one or more elution steps are performed sequentially to elute nucleic acids having different levels of methylation. For example, a first set of methylated nucleic acids can be eluted at a salt concentration of 160 mM or higher, e.g., at least 150 mM, at least 200 mM, 300 mM, 400 mM, 500 mM, 600 mM, 700 mM, 800 mM, 900 mM, 1000 mM, or 2000 mM. After such methylated nucleic acids are eluted, magnetic separation is once again used to separate higher level of methylated nucleic acids from those with lower level of methylation. The elution and magnetic separation steps can be repeated to create various partitions such as a hypomethylated partition (enriched in nucleic acids comprising no methylation), a methylated partition (enriched in nucleic acids comprising low levels of methylation), and a hyper methylated partition (enriched in nucleic acids comprising high levels of methylation).

In some methods, nucleic acids bound to an agent used for affinity separation-based partitioning are subjected to a wash step. The wash step washes off nucleic acids weakly bound to the affinity agent. Such nucleic acids can be enriched in nucleic acids having the modification to an extent close to the mean or median (i.e., intermediate between nucleic acids remaining bound to the solid phase and nucleic acids not binding to the solid phase on initial contacting of the sample with the agent).

The affinity separation results in at least two, and sometimes three or more partitions of nucleic acids with different extents of a modification. While the partitions are still separate, the nucleic acids of at least one partition, and usually two or three (or more) partitions are linked to nucleic acid tags, usually provided as components of adapters, with the nucleic acids in different partitions receiving different tags that distinguish members of one partition from another. The tags linked to nucleic acid molecules of the same partition can be the same or different from one another. But if different from one another, the tags may have part of their code in common so as to identify the molecules to which they are attached as being of a particular partition.

For further details regarding portioning nucleic acid samples based on characteristics such as methylation, see WO2018/119452, which is incorporated herein by reference.

In some embodiments, the nucleic acid molecules can be partitioned into different partitions based on the nucleic acid molecules that are bound to a specific protein or a fragment thereof and those that are not bound to that specific protein or fragment thereof.

Nucleic acid molecules can be partitioned based on DNA-protein binding. Protein-DNA complexes can be partitioned based on a specific property of a protein. Examples of such properties include various epitopes, modifications (e.g., histone methylation or acetylation) or enzymatic activity. Examples of proteins which may bind to DNA and serve as a basis for fractionation may include, but are not limited to, protein A and protein G. Any suitable method can be used to partition the nucleic acid molecules based on protein bound regions. Examples of methods used to partition nucleic acid molecules based on protein bound regions include, but are not limited to, SDS-PAGE, chromatin-immuno-precipitation (ChIP), heparin chromatography, and asymmetrical field flow fractionation (AF4).

In some embodiments, the partitioning comprises contacting the DNA with a methylation sensitive restriction enzyme (MSRE) and/or a methylation dependent restriction enzyme (MDRE). Following the treatment of the DNA with a MSRE or a MDRE, the DNA may be partitioned based on size to generate hypermethylated (longest DNA molecules following MSRE treatment and shortest DNA fragments following MDRE treatment), intermediate (intermediate length DNA molecules following MSRE or MDRE treatment), and hypomethylated (shortest DNA molecules following MSRE treatment and longest DNA fragments following MDRE treatment) subsamples.

In some embodiments, the partitioning is performed by contacting the nucleic acids with a methyl binding domain (“MBD”) of a methyl binding protein (“MBP”). In some such embodiments, the nucleic acids are contacted with an entire MBP. In some embodiments, an MBD binds to 5-methylcytosine (5mC), and an MBP comprises an MBD and is referred to interchangeably herein as a methyl binding protein or a methyl binding domain protein. In some embodiments, MBD is coupled to paramagnetic beads, such as Dynabeads® M-280 Streptavidin via a biotin linker. Partitioning into fractions with different extents of methylation can be performed by eluting fractions by increasing the NaCl concentration.

In some embodiments, bound DNA is eluted by contacting the antibody or MBD with a protease, such as proteinase K. This may be performed instead of or in addition to elution steps using NaCl as discussed above.

Examples of agents that recognize a modified nucleobase contemplated herein include, but are not limited to:
(a) MeCP2 is a protein that preferentially binds to 5-methyl-cytosine over unmodified cytosine.
(b) RPL26, PRP8 and the DNA mismatch repair protein MHS6 preferentially bind to 5-hydroxymethyl-cytosine over unmodified cytosine.
(c) FOXK1, FOXK2, FOXP1, FOXP4 and FOXI3 preferably bind to 5-formyl-cytosine over unmodified cytosine (Iurlaro et al., Genome Biol. 14: R119 (2013)).
(d) Antibodies specific to one or more methylated or modified nucleobases or conversion products thereof, such as 5mC, 5caC, or DHU.

In general, elution is a function of the number of modifications, such as the number of methylated sites per molecule, with molecules having more methylation eluting under increased salt concentrations. To elute the DNA into distinct populations based on the extent of methylation, one can use a series of elution buffers of increasing NaCl concentration. Salt concentration can range from about 100 nm to about 2500 mM NaCl. In one embodiment, the process results in three (3) partitions. Molecules are contacted with a solution at a first salt concentration and comprising a molecule comprising an agent that recognizes a modified nucleobase, which molecule can be attached to a capture moiety, such as streptavidin. At the first salt concentration a population of molecules will bind to the agent and a population will remain unbound. The unbound population can be separated as a “hypomethylated” population. For example, a first partition enriched in hypomethylated form of DNA is that which remains unbound at a low salt concentration, e.g., 100 mM or 160 mM. A second partition enriched in intermediate methylated DNA is eluted using an intermediate salt concentration, e.g., between 100 mM and 2000 mM concentration. This is also separated from the sample. A third partition enriched in hypermethylated form of DNA is eluted using a high salt concentration, e.g., at least about 2000 mM.

In some embodiments, a monoclonal antibody raised against 5-methylcytidine (5mC) is used to purify methylated DNA. DNA is denatured, e.g., at 95 °C in order to yield single-stranded DNA fragments. Protein G coupled to standard or magnetic beads as well as washes following incubation with the anti-5mC antibody are used to immunoprecipitate DNA bound to the antibody. Such DNA may then be eluted. Partitions may comprise unprecipitated DNA and one or more partitions eluted from the beads.

In some embodiments, the partitions of DNA are desalted and concentrated in preparation for enzymatic steps of library preparation.

Sequences that comprise aberrantly high copy numbers may tend to be hypermethylated. Accordingly, in some embodiments, the DNA contacted with capture probes specific for members of an epigenetic target region set comprising a plurality of target regions that are both type-specific differentially methylated regions and copy number variants comprises at least a portion of a hypermethylated partition. The DNA from or comprising at least a portion of the hypermethylated partition may or may not be combined with DNA from or comprising at least a portion of one or more other partitions, such as an intermediate partition or a hypomethylated partition.

### Amplification

Adapted DNA can be amplified (e.g. by PCR) prior to, or as part of, the modification-sensitive sequencing. For example, in modification-sensitive sequencing procedures which comprise a conversion step, the adapted DNA may be amplified after the conversion step. In modification-sensitive sequencing procedures which involve single molecule sequencing (such a nanopore-based sequencing or SMRT sequencing), there may be no amplification step.

Amplification is typically primed by primers binding to primer binding sites in adapters flanking a DNA molecule to be amplified. Amplification methods can involve cycles of denaturation, annealing and extension, resulting from thermocycling or can be isothermal as in transcription-mediated amplification. Other amplification methods include the ligase chain reaction, strand displacement amplification, nucleic acid sequence based amplification, and self-sustained sequence based replication.

In some embodiments, the present methods perform dsDNA ligations with T-tailed and C-tailed adapters. The addition of C-tailed adapters can increase ligation efficiency because the A-tailing reaction can also add G-tails to a small portion of the DNA molecules, when the A tailing is performed in the presence of dGTP, such as when the A-tailing is performed in the same reaction as the end repair. The use of T-tailed and C-tailed adapters can result in amplification of at least 50, 60, 70 or 80% of double stranded nucleic acids. Preferably the present methods increase the amount or number of amplified molecules relative to control methods performed with T-tailed adapters alone by at least 10, 15 or 20%.

In some embodiments, adapted DNA is amplified before sequencing. Amplification may in some cases be before one or more capture steps. In some embodiments, the ligation step occurs after the conversion step. In some embodiments, the ligation occurs before or simultaneously with amplification.

1. Pooling of DNA from Samples or Subsamples or Portions Thereof

In some embodiments, the methods herein comprise preparing one or more pools comprising tagged DNA from a plurality of partitioned subsamples. In some embodiments, a pool comprises at least a portion of the DNA of a hypomethylated partition and at least a portion of the DNA of a hypermethylated partition. Target regions, e.g., including epigenetic target regions and/or sequence-variable target regions, may be captured from a pool. The steps of capturing a target region set from at least an aliquot or portion of a sample or subsample described elsewhere herein can encompass capture steps performed on a pool comprising DNA from first and second subsamples. A step of amplifying DNA in a pool may be performed before capturing target regions from the pool. The capturing step may have any of the features described for capturing steps elsewhere herein.

In some embodiments, the methods comprise preparing a first pool comprising at least a portion of the DNA of a hypomethylated partition. In some embodiments, the methods comprise preparing a second pool comprising at least a portion of the DNA of a hypermethylated partition. In some embodiments, the methods comprise capturing at least a first set of target regions from the first pool, wherein the first set comprises sequence-variable target regions. A step of amplifying DNA in the first pool may be performed before this capture step. In some embodiments, capturing the first set of target regions from the first pool comprises contacting the DNA of the first pool with a first set of target-specific probes, wherein the first set of target-specific probes comprises target-binding probes specific for the sequence-variable target regions. In some embodiments, the methods comprise capturing a second plurality of sets of target regions from the second pool, wherein the second plurality comprises sequence-variable target regions and epigenetic target regions. A step of amplifying DNA in the second pool may be performed before this capture step. In some embodiments, capturing the second plurality of sets of target regions from the second pool comprises contacting the DNA of the first pool with a second set of target-specific probes, wherein the second set of target-specific probes comprises target-binding probes specific for the sequence-variable target regions and target-binding probes specific for the epigenetic target regions.

In some embodiments, sequence-variable target regions are captured from a second portion of a partitioned subsample. The second portion may include some, a majority, substantially all, or all of the DNA of the subsample that was not included in the pool. The regions captured from the pool and from the subsample may be combined and analyzed in parallel.

The epigenetic target regions may show differences in methylation levels and/or fragmentation patterns depending on whether they originated from a particular cell or tissue type or from a tumor or from healthy cells, as discussed elsewhere herein. The sequence-variable target regions may show differences in sequence depending on whether they originated from a tumor or from healthy cells.

Analysis of epigenetic target regions from a hypomethylated partition may be less informative in some applications than analysis of sequence-variable target regions from hypermethylated and hypomethylated partitions and epigenetic target regions from a hypermethylated partition. As such, in methods where sequence-variable target regions and epigenetic target regions are being captured, the latter may be captured to a lesser extent than one or more of the sequence-variable target regions are captured from the hypermethylated and hypomethylated partitions and/or to a lesser extent that epigenetic target regions are captured from a hypermethylated partition. For example, sequence-variable target regions can be captured from a portion of a hypomethylated partition that is not pooled with a hypermethylated partition, and the pool can be prepared with some (e.g., a majority, substantially all, or all) of the DNA from a hypermethylated partition and none or some (e.g., a minority) of the DNA from a hypomethylated partition. Such approaches can reduce or eliminate sequencing of epigenetic target regions from hypomethylated partitions, thereby reducing the amount of sequencing data that suffices for further analysis.

In some embodiments, including a minority of the DNA of a hypomethylated partition in the pool facilitates quantification of one or more epigenetic features (e.g., methylation or other epigenetic feature(s) discussed in detail elsewhere herein), e.g., on a relative basis.

In some embodiments, the pool comprises a minority of the DNA of a hypomethylated partition, e.g., less than about 50% of the DNA of a hypomethylated partition, such as less than or equal to about 45%, 40%, 35%, 30%, 25%, 20%, 15%, 10%, or 5% of the DNA of a hypomethylated partition. In some embodiments, the pool comprises about 5%-25% of the DNA of a hypomethylated partition. In some embodiments, the pool comprises about 10%-20% of the DNA of a hypomethylated partition. In some embodiments, the pool comprises about 10% of the DNA of a hypomethylated partition. In some embodiments, the pool comprises about 15% of the DNA of a hypomethylated partition. In some embodiments, the pool comprises about 20% of the DNA of a hypomethylated partition.

In some embodiments, the pool comprises a portion of a hypermethylated partition, which may be at least about 50% of the DNA of a hypermethylated partition. For example, the pool may comprise at least about 55%, 60%, 65%, 70%, 75%, 80%, 85%, 90%, or 95% of the DNA of a hypermethylated partition. In some embodiments, the pool comprises 50-55%, 55-60%, 60-65%, 65-70%, 70-75%, 75-80%, 80-85%, 85-90%, 90-95%, or 95-100% of the DNA of a hypermethylated partition. In some embodiments, the second pool comprises all or substantially all of the DNA of a hypermethylated partition.

In some embodiments, a first pool comprises substantially all or all of the DNA of a hypomethylated partition (e.g., wherein a second pool does not comprise DNA of a hypomethylated partition. In some embodiments, the second pool does not comprise DNA of a hypomethylated partition (e.g., wherein the first pool comprises substantially all or all of the DNA of a hypomethylated partition).

In some embodiments, a second pool comprises a portion of a hypermethylated partition, which may be any of the values and ranges set forth above with respect to a hypomethylated partition. In some embodiments, the second pool comprises all or substantially all of the DNA of a hypermethylated partition.

In an exemplary embodiment, after partitioning, the partitions separately undergo end repair and ligation to adapters comprising molecular barcodes and are then amplified separately. After the amplification, amplified molecules are enriched (still keeping the partitions separate). Post-enrichment, the enriched DNA are pooled according to any of the embodiments described herein, and then amplified again. After amplification, the molecules are sequenced.

In various embodiments, the methods further comprise sequencing the captured DNA, e.g., to different degrees of sequencing depth for the epigenetic and sequence-variable target region sets, consistent with the discussion herein.

### L. Enriching, Capturing and Using Capture Probes

DNA molecules in a sample can be subject to a capture step, in which molecules having target sequences are captured for subsequent analysis. In some embodiments, methods disclosed herein comprise a step of capturing one or more sets of target regions of DNA, such as cfDNA or DNA from a sample comprising cells (such as a blood sample). Capture may be performed using any suitable approach known in the art. Target capture can involve use of a bait set comprising oligonucleotide baits labeled with a capture moiety, such as biotin or the other examples noted below. The probes can have sequences selected to tile across a panel of regions, such as genes. Such bait sets are combined with a sample under conditions that allow hybridization of the target molecules with the baits. Then, captured molecules are isolated using the capture moiety. For example, a biotin capture moiety by bead-based streptavidin. Such methods are further described in, for example, U.S. Pat. No. 9,850,523, issuing Dec. 26, 2017, which is incorporated herein by reference.

In some embodiments of the disclosed methods, a capturing step occurs prior to a step of sequencing by synthesis and prior to a step of performing a conversion procedure on a DNA molecule (such as on an adapted DNA molecule). In some embodiments of the disclosed methods, a capturing step occurs prior to a step of sequencing by synthesis and after a step of performing a conversion procedure on a DNA molecule (such as on an adapted DNA molecule). In some embodiments, a capturing step occurs prior to a step of sequencing by synthesis and prior to a step of tagging a DNA molecule. In some embodiments, a capturing step occurs prior to a step of sequencing by synthesis and after a step of tagging a DNA molecule. In some embodiments, capturing can be performed before a step of ligating adapters (such as Y-shaped adapters as disclosed herein) to DNA molecules, e.g., to facilitate including partition tags in the one or more captured sets of target DNA. In other embodiments, capturing can be performed after a step of ligating adapters (such as Y-shaped adapters as disclosed herein) to DNA molecules. In some embodiments, a capturing step occurs prior to a partitioning step as disclosed herein. In some embodiments, a capturing step occurs after a partitioning step as disclosed herein.

Capture moieties include, without limitation, biotin, avidin, streptavidin, a nucleic acid comprising a particular nucleotide sequence, a hapten recognized by an antibody, and magnetically attractable particles. The extraction moiety can be a member of a binding pair, such as biotin/streptavidin or hapten/antibody. In some embodiments, a capture moiety that is attached to an analyte is captured by its binding pair which is attached to an isolatable moiety, such as a magnetically attractable particle or a large particle that can be sedimented through centrifugation. The capture moiety can be any type of molecule that allows affinity separation of nucleic acids bearing the capture moiety from nucleic acids lacking the capture moiety. Exemplary capture moieties are biotin which allows affinity separation by binding to streptavidin linked or linkable to a solid phase or an oligonucleotide, which allows affinity separation through binding to a complementary oligonucleotide linked or linkable to a solid phase.

A panel of regions targeted for enrichment can be selected such that they do not contain regions known to include the base modification used in the end repair reaction. When the end repair is performed with dNTPs comprising 5mC or 5hmC, a panel of regions targeted for enrichment may be selected such that they do not contain CpH dinucleotides which are known to be naturally methylated in the subject (e.g. humans). Such CpH dinucleotides can be identified through the use of publicly available resources (e.g. MethBank3.0: a database of DNA methylomes across a variety of species Nucleic Acids Res 2018). Such an approach has the advantage that any detected methylated CpH dinucleotides can unambiguously be attributed to regions synthesized in the end repair.

In some embodiments, capturing comprises contacting the DNA to be captured with a set of target-specific probes. The set of target-specific probes may have any of the features described herein for sets of target-specific probes, including but not limited to in the embodiments set forth above and the sections relating to probes below. Capturing may be performed on one or more subsamples prepared during methods disclosed herein. In some embodiments, DNA is captured from at least the first subsample or the second subsample, e.g., at least the first subsample and the second subsample. In some embodiments, the subsamples are differentially tagged (e.g., as described herein) and then pooled before undergoing capture.

The capturing step may be performed using conditions suitable for specific nucleic acid hybridization, which generally depend to some extent on features of the probes such as length, base composition, etc. Those skilled in the art will be familiar with appropriate conditions given general knowledge in the art regarding nucleic acid hybridization. In some embodiments, complexes of target-specific probes and DNA are formed.

In some embodiments, a method described herein comprises capturing cfDNA obtained from a subject for a plurality of sets of target regions. The target regions comprise epigenetic target regions, which may show differences in methylation levels and/or fragmentation patterns depending on whether they originated from a tumor or from healthy cells. The target regions also comprise sequence-variable target regions, which may show differences in sequence depending on whether they originated from a tumor or from healthy cells. The capturing step produces a captured set of cfDNA molecules, and the cfDNA molecules corresponding to the sequence-variable target region set are captured at a greater capture yield in the captured set of cfDNA molecules than cfDNA molecules corresponding to the epigenetic target region set. For additional discussion of capturing steps, capture yields, and related aspects, see WO2020/160414, which is incorporated herein by reference for all purposes.

In some embodiments, a method described herein comprises contacting cfDNA obtained from a subject with a set of target-specific probes, wherein the set of target-specific probes is configured to capture cfDNA corresponding to the sequence-variable target region set at a greater capture yield than cfDNA corresponding to the epigenetic target region set.

It can be beneficial to capture cfDNA corresponding to the sequence-variable target region set at a greater capture yield than cfDNA corresponding to the epigenetic target region set because a greater depth of sequencing may be necessary to analyze the sequence-variable target regions with sufficient confidence or accuracy than may be necessary to analyze the epigenetic target regions. The volume of data needed to determine fragmentation patterns (e.g., to test fsor perturbation of transcription start sites or CTCF binding sites) or fragment abundance (e.g., in hypermethylated and hypomethylated partitions) is generally less than the volume of data needed to determine the presence or absence of cancer-related sequence mutations. Capturing the target region sets at different yields can facilitate sequencing the target regions to different depths of sequencing in the same sequencing run (e.g., using a pooled mixture and/or in the same sequencing cell).

In various embodiments, the methods further comprise sequencing the captured cfDNA, e.g., to different degrees of sequencing depth for the epigenetic and sequence-variable target region sets, consistent with the discussion herein.

In some embodiments, complexes of target-specific probes and DNA are separated from DNA not bound to target-specific probes. For example, where target-specific probes are bound covalently or noncovalently to a solid support, a washing or aspiration step can be used to separate unbound material. Alternatively, where the complexes have chromatographic properties distinct from unbound material (e.g., where the probes comprise a ligand that binds a chromatographic resin), chromatography can be used.

As discussed in detail elsewhere herein, the set of target-specific probes may comprise a plurality of sets such as probes for a sequence-variable target region set and probes for an epigenetic target region set. In some such embodiments, the capturing step is performed with the probes for the sequence-variable target region set and the probes for the epigenetic target region set in the same vessel at the same time, e.g., the probes for the sequence-variable and epigenetic target region sets are in the same composition. This approach provides a relatively streamlined workflow. In some embodiments, the concentration of the probes for the sequence-variable target region set is greater than the concentration of the probes for the epigenetic target region set.

Alternatively, the capturing step is performed with the sequence-variable target region probe set in a first vessel and with the epigenetic target region probe set in a second vessel, or the contacting step is performed with the sequence-variable target region probe set at a first time and a first vessel and the epigenetic target region probe set at a second time before or after the first time. This approach allows for preparation of separate first and second compositions comprising captured DNA corresponding to the sequence-variable target region set and captured DNA corresponding to the epigenetic target region set. The compositions can be processed separately as desired (e.g., to fractionate based on methylation as described elsewhere herein) and recombined in appropriate proportions to provide material for further processing and analysis such as sequencing.

In some embodiments, a captured set of DNA (e.g., cfDNA) is provided. With respect to the disclosed methods, the captured set of DNA may be provided, e.g., by performing a capturing step prior to a sequencing step as described herein. The captured set may comprise DNA corresponding to a sequence-variable target region set, an epigenetic target region set, or a combination thereof. In some embodiments, a capture step is performed prior to a conversion step or after a conversion step.

In some embodiments, a first target region set is captured (e.g., from a sample or a first subsample), comprising at least epigenetic target regions. The epigenetic target regions captured from the first subsample may comprise hypermethylation variable target regions. In some embodiments, the hypermethylation variable target regions are CpG-containing regions that are unmethylated or have low methylation in cfDNA from healthy subjects (e.g., below-average methylation relative to bulk cfDNA). In some embodiments, the hypermethylation variable target regions are regions that show lower methylation in healthy cfDNA than in at least one other tissue type. Without wishing to be bound by any particular theory, cancer cells may shed more DNA into the bloodstream than healthy cells of the same tissue type. As such, the distribution of tissue of origin of cfDNA may change upon carcinogenesis. Thus, an increase in the level of hypermethylation variable target regions in the first subsample can be an indicator of the presence (or recurrence, depending on the history of the subject) of cancer.

In some embodiments, a second target region set is captured from the second subsample, comprising at least epigenetic target regions. The epigenetic target regions may comprise hypomethylation variable target regions. In some embodiments, the hypomethylation variable target regions are CpG-containing regions that are methylated or have high methylation in cfDNA from healthy subjects (e.g., above-average methylation relative to bulk cfDNA). In some embodiments, the hypomethylation variable target regions are regions that show higher methylation in healthy cfDNA than in at least one other tissue type. Without wishing to be bound by any particular theory, cancer cells may shed more DNA into the bloodstream than healthy cells of the same tissue type. As such, the distribution of tissue of origin of cfDNA may change upon carcinogenesis. Thus, an increase in the level of hypomethylation variable target regions in the second subsample can be an indicator of the presence (or recurrence, depending on the history of the subject) of cancer.

In some embodiments the quantity of captured sequence-variable target region DNA is greater than the quantity of the captured epigenetic target region DNA, when normalized for the difference in the size of the targeted regions (footprint size).

Alternatively, first and second captured sets may be provided, comprising, respectively, DNA corresponding to a sequence-variable target region set and DNA corresponding to an epigenetic target region set. The first and second captured sets may be combined to provide a combined captured set.

In some embodiments in which a captured set comprising DNA corresponding to the sequence-variable target region set and the epigenetic target region set includes a combined captured set as discussed above, the DNA corresponding to the sequence-variable target region set may be present at a greater concentration than the DNA corresponding to the epigenetic target region set, e.g., a 1.1 to 1.2-fold greater concentration, a 1.2- to 1.4-fold greater concentration, a 1.4- to 1.6-fold greater concentration, a 1.6- to 1.8-fold greater concentration, a 1.8- to 2.0-fold greater concentration, a 2.0- to 2.2-fold greater concentration, a 2.2- to 2.4-fold greater concentration a 2.4- to 2.6-fold greater concentration, a 2.6- to 2.8-fold greater concentration, a 2.8- to 3.0-fold greater concentration, a 3.0- to 3.5-fold greater concentration, a 3.5- to 4.0, a 4.0- to 4.5-fold greater concentration, a 4.5- to 5.0-fold greater concentration, a 5.0-to 5.5-fold greater concentration, a 5.5- to 6.0-fold greater concentration, a 6.0- to 6.5-fold greater concentration, a 6.5- to 7.0-fold greater, a 7.0- to 7.5-fold greater concentration, a 7.5- to 8.0-fold greater concentration, an 8.0- to 8.5-fold greater concentration, an 8.5- to 9.0-fold greater concentration, a 9.0- to 9.5-fold greater concentration, 9.5- to 10.0-fold greater concentration, a 10- to 11-fold greater concentration, an 11- to 12-fold greater concentration a 12- to 13-fold greater concentration, a 13- to 14-fold greater concentration, a 14- to 15-fold greater concentration, a 15- to 16-fold greater concentration, a 16- to 17-fold greater concentration, a 17- to 18-fold greater concentration, an 18- to 19-fold greater concentration, a 19- to 20-fold greater concentration, a 20- to 30-fold greater concentration, a 30- to 40-fold greater concentration, a 40- to 50-fold greater concentration, a 50- to 60-fold greater concentration, a 60- to 70-fold greater concentration, a 70- to 80-fold greater concentration, a 80-to 90-fold greater concentration, or a 90- to 100-fold greater concentration. The degree of difference in concentrations accounts for normalization for the footprint sizes of the target regions, as discussed in the definition section.

In some embodiments, the DNA that is captured comprises intronic regions. In some embodiments, the intronic regions comprise one or more introns likely to differentiate DNA from neoplastic (e.g., tumor or cancer) cells and from healthy cells, e.g., non-neoplastic circulating cells. For example, an intron comprising a rearrangement known to be present in some neoplastic cells and absent from healthy cells can be used to differentiate DNA from neoplastic (e.g., tumor or cancer) cells and from healthy cells. In some embodiments, the rearrangement is a translocation.

In some embodiments, captured intronic regions have a footprint of at least 30 bp, e.g., at least 100 bp, at least 200 bp, at least 500 bp, at least 1 kb, at least 2 kb, at least 5 kb, at least 10 kb, at least 20 kb, at least 50 kb, at least 200 kb, at least 300 kb, or at least 400 kb. In some embodiments, the intronic target region set has a footprint in the range of 30 bp-1000 kb, e.g., 30 bp-100 bp, 100 bp-200 bp, 200 bp-500 bp, 500 bp-1 kb, 1 kb-2 kb, 2 kb-5 kb, 5 kb-10 kb, 10 kb-20 kb, 20 kb-50 kb, 50 kb-100 kb, 100-200 kb, 200-300 kb, 300-400 kb, 400-500 kb, 500-600 kb, 600-700 kb, 700-800 kb, 800-900 kb, and 900-1,000 kb.

Exemplary rearrangements, such as intronic translocations that can be detected using the methods described herein include but are not limited to translocations wherein at least one of the two genes involved in the translocation is a receptor tyrosine kinase. Exemplary translocation products are the BCR-ABL fusion. and fusions comprising any of ALK, FGFR2, FGFR3, NTRK1, RET, or ROS1.

In some embodiments, the DNA that is captured comprises target regions having a type-specific epigenetic variation. In some embodiments, an epigenetic target region set consists of target regions having a type-specific epigenetic variation. In some embodiments, the type-specific epigenetic variations, e.g., differential methylation or a type-specific fragmentation pattern, are likely to differentiate DNA from one or more related cell or tissue types cells from DNA from other cell or tissue types present in a sample or in a subject.

In some embodiments, nucleic acids captured or enriched using a method described herein comprise captured DNA, such as one or more captured sets of DNA. In some embodiments, the captured DNA comprise target regions that are differentially methylated in different cell types (such as different immune cell types). In some embodiments wherein the different cell types comprise different immune cell types, the immune cell types comprise rare or closely related immune cell types, such as activated and naïve lymphocytes or myeloid cells at different stages of differentiation.

In some embodiments, a captured epigenetic target region set captured from a sample or first subsample comprises hypermethylation variable target regions. In some embodiments, the hypermethylation variable target regions are differentially or exclusively hypermethylated in one or more related cell or tissue types. In some embodiments, the hypermethylation variable target regions are differentially or exclusively hypermethylated in one cell type (such as in one cancer cell type and/or in one immune cell type). In some embodiments, the hypermethylation variable target regions are hypermethylated to an extent that is distinguishably higher or exclusively present in one cell type (such as in one cancer cell type and/or in one immune cell type). Such hypermethylation variable target regions may be hypermethylated in other cell or tissue types but not to the extent observed in the one or more related cell or tissue types. In some embodiments, the hypermethylation variable target regions show lower methylation in healthy cfDNA than in at least one other tissue type. In some embodiments, the hypermethylation variable target regions show even higher methylation in cfDNA from a diseased cell of the one or more related cell or tissue types. In some embodiments, target regions comprise hypermethylated regions with aberrantly high copy number. In some such embodiments, the target regions are hypermethylated in healthy and diseased colon tissue and have aberrantly high copy number in pre-cancerous or cancerous colon tissue. Examples of such target regions are shown in Table 1 below. A gene is considered to comprise a DMR when the DMR is located within an untranslated region (UTR), intron, or exon of the gene, or within 5000 nucleotides of either the 5′ end of the sense strand of the 5′ UTR or the 3′ end of the sense strand of the 3′ UTR.

TABLE 1
Hypermethylated target regions with aberrantly
high copy number in colon cancer or pre-cancer

| Chromosomal region | |
|---|---|
| with copy number gain | Genes comprising DMRs within the chromosomal region |
| 7p21.3-15.1 | VWDE, TWIST1, DNAH1, GPNMB, NPY, GSDME, NFE2L3, |
| | HOXA1, HOXA7, EVX1, CREB5 |
| 8q13.3-24.3 | PRDM14, MSC, TRPA1, SBSPON, CRISPLD1, ZFHX4, RALYL, |
| | MMP16, C8orf88, RUNX1T1, CDH17, GDF6, SDC2, OSR2, |
| | PABPC1, ZNF706, RIMS2, ZFPM2, SYBU, CSMD3, TRPS1, |
| | AARD, TNFRSF11B, MAL2, COL14A1, POU5F1B, ADCY8, |
| | ST3GAL1, TRAPPC9, AGO2, ADGRB1, LY6E, LY6H, GLI4, |
| | MAFA, TSTA3, PLEC, OPLAH, FOXH1 |
| 13q14.1-33.3 | ELF1, RGCC, TNFSF11, SERP2, RB1, CNMD, PCDH17, EDNRB, |
| | POU4F1, SLITRK5, GPC6, SOX21, CLDN10, STK24, ZIC2, |
| | NALCN, FGF14, MYO16, COL4A1, COL4A2, SOX1, F7 |
| 20q12-13.33 | MAFB, LPIN3, EMILIN3, PTPRT, TOX2, GDAPIL1, HNF4A, |
| | MMP9, CDH22, PREX1, ZNFX1, PARD6B, ZFP64, DOK5, |
| | FAM210B, TFAP2C, RBM38, CTCFL, GNAS, PHACTR3, CDH4, |
| | GATA5, BHLHE23, YTHDF1, NKAIN4, CHRNA4, KCNQ2, |
| | GMEB2 |

TABLE 2
Exemplary Hypermethylation Target Regions
based on Lung Cancer studies

| Gene Name | Chromosome |
|---|---|
| MARCH11 | chr5 |
| TAC1 | chr7 |
| TCF21 | chr6 |
| SHOX2 | chr3 |
| p16 | chr3 |
| Casp8 | chr2 |
| CDH13 | chr16 |
| MGMT | chr10 |
| MLH1 | chr3 |
| MSH2 | chr2 |
| TSLC1 | chr11 |
| APC | chr5 |
| DKK1 | chr10 |
| DKK3 | chr11 |
| LKB1 | chr11 |
| WIF1 | chr12 |
| RUNX3 | chr1 |
| GATA4 | chr8 |
| GATA5 | chr20 |
| PAX5 | chr9 |
| E-Cadherin | chr16 |
| H-Cadherin | chr16 |

In some embodiments, a captured epigenetic target region set captured from a sample or subsample comprises hypomethylation variable target regions. In some embodiments, the hypomethylation variable target regions are exclusively hypomethylated in one or more related cell or tissue types. In some embodiments, the hypomethylation variable target regions are exclusively hypomethylated in one cell type (such as in one cancer cell type and/or in one immune cell type). In some embodiments, the hypomethylation variable target regions are hypomethylated to an extent that is exclusively present in one cell type (such as in one cancer cell type and/or in one immune cell type). Such hypomethylation variable target regions may be hypomethylated in other cell or tissue types but not to the extent observed in the one or more cell or tissue types. In some embodiments, the hypomethylation variable target regions show higher methylation in healthy cfDNA than in at least one other tissue type.

Without wishing to be bound by any particular theory, in an individual with cancer, proliferating or activated immune cells and/or dying cancer cells may shed more DNA into the bloodstream than immune cells in a healthy individual and/or healthy cells of the same tissue type, respectively. As such, the distribution of cell type and/or tissue of origin of cfDNA may change upon carcinogenesis. Thus, the presence and/or levels of cfDNA originating from certain cell or tissue types can be an indicator of disease. Variations in hypermethylation and/or hypomethylation can be an indicator of disease. For example, an increase in the level of hypermethylation variable target regions and/or hypomethylation variable target regions in a subsample following a partitioning step can be an indicator of the presence (or recurrence, depending on the history of the subject) of cancer.

Exemplary hypermethylation variable target regions and hypomethylation variable target regions useful for distinguishing between various cell types, including but not limited to cancer cell types and non-cancer cell types, and/or immune cell types, have been identified by analyzing DNA obtained from various cell types via whole genome bisulfite sequencing, as described, e.g., in Scott, C. A., Duryea, J. D., MacKay, H. et al., “Identification of cell type-specific methylation signals in bulk whole genome bisulfite sequencing data,” Genome Biol 21, 156 (2020) (doi.org/10.1186/si3059-020-02065-5). Whole-genome bisulfite sequencing data is available from the Blueprint consortium, available on the internet at dcc.blueprint-epigenome.eu.

In some embodiments, first and second captured target region sets comprise, respectively, DNA corresponding to a sequence-variable target region set and DNA corresponding to an epigenetic target region set, for example, as described in WO 2020/160414. The first and second captured sets may be combined to provide a combined captured set. The sequence-variable target region set and epigenetic target region set may have any of the features described for such sets in WO 2020/160414, which is incorporated by reference herein in its entirety. In some embodiments, the epigenetic target region set comprises a hypermethylation variable target region set. In some embodiments, the epigenetic target region set comprises a hypomethylation variable target region set. In some embodiments, the epigenetic target region set comprises CTCF binding regions. In some embodiments, the epigenetic target region set comprises fragmentation variable target regions. In some embodiments, the epigenetic target region set comprises transcriptional start sites. In some embodiments, the epigenetic target region set comprises regions that may show focal amplifications in cancer, e.g., one or more of AR, BRAF, CCND1, CCND2, CCNE1, CDK4, CDK6, EGFR, ERBB2, FGFR1, FGFR2, KIT, KRAS, MET, MYC, PDGFRA, PIK3CA, and RAF1. For example, in some embodiments, the epigenetic target region set comprises at least 2, 3, 4, 5, 6, 7, 8, 9, 10, 11, 12, 13, 14, 15, 16, 17, or 18 of the foregoing targets.

In some embodiments, the sequence-variable target region set comprises a plurality of regions known to undergo somatic mutations in cancer. In some aspects, the sequence-variable target region set targets a plurality of different genes or genomic regions (“panel”) selected such that a determined proportion of subjects having a cancer exhibits a genetic variant or tumor marker in one or more different genes or genomic regions in the panel. The panel may be selected to limit a region for sequencing to a fixed number of base pairs. The panel may be selected to sequence a desired amount of DNA, e.g., by adjusting the affinity and/or amount of the probes as described elsewhere herein. The panel may be further selected to achieve a desired sequence read depth. The panel may be selected to achieve a desired sequence read depth or sequence read coverage for an amount of sequenced base pairs. The panel may be selected to achieve a theoretical sensitivity, a theoretical specificity, and/or a theoretical accuracy for detecting one or more genetic variants in a sample.

Probes for detecting the panel of regions can include those for detecting genomic regions of interest (hotspot regions). Information about chromatin structure can be taken into account in designing probes, and/or probes can be designed to maximize the likelihood that particular sites (e.g., KRAS codons 12 and 13) can be captured, and may be designed to optimize capture based on analysis of cfDNA coverage and fragment size variation impacted by nucleosome binding patterns and GC sequence composition. Regions used herein can also include non-hotspot regions optimized based on nucleosome positions and GC models.

Probes for detecting the panel of regions can include those for detecting genomic regions of interest (hotspot regions). Information about chromatin structure can be taken into account in designing probes, and/or probes can be designed to maximize the likelihood that particular sites (e.g., KRAS codons 12 and 13) can be captured, and may be designed to optimize capture based on analysis of cfDNA coverage and fragment size variation impacted by nucleosome binding patterns and GC sequence composition. Regions used herein can also include non-hotspot regions optimized based on nucleosome positions and GC models.

Examples of listings of genomic locations of interest may be found in Table 3 and Table 4 of WO 2020/160414. In some embodiments, a sequence-variable target region set used in the methods of the present disclosure comprises at least a portion of at least 5, at least 10, at least 15, at least 20, at least 25, at least 30, at least 35, at least 40, at least 45, at least 50, at least 55, at least 60, at least 65, or 70 of the genes of Table 3 of WO 2020/160414. In some embodiments, a sequence-variable target region set used in the methods of the present disclosure comprises at least a portion of at least 5, at least 10, at least 15, at least 20, at least 25, at least 30, at least 35, at least 40, at least 45, at least 50, at least 55, at least 60, at least 65, at least 70, or 73 of the genes of Table 4 of WO 2020/160414. Additionally or alternatively, suitable target region sets are available from the literature. For example, Gale et al., PLoS One 13: e0194630 (2018), which is incorporated herein by reference, describes a panel of 35 cancer-related gene targets that can be used as part or all of a sequence-variable target region set. These 35 targets are AKT1, ALK, BRAF, CCND1, CDK2A, CTNNB1, EGFR, ERBB2, ESR1, FGFR1, FGFR2, FGFR3, FOXL2, GATA3, GNA11, GNAQ, GNAS, HRAS, IDH1, IDH2, KIT, KRAS, MED12, MET, MYC, NFE2L2, NRAS, PDGFRA, PIK3CA, PPP2R1A, PTEN, RET, STK11, TP53, and U2AF1.

In some embodiments, the sequence-variable target region set comprises target regions from at least 10, 20, 30, or 35 cancer-related genes, such as the cancer-related genes listed above and in WO 2020/160414.

In some embodiments, a collection of capture probes is used in methods described herein, e.g., comprising capture probes prepared by any method disclosed herein for doing so. In some embodiments, the collection of capture probes further comprises target-binding probes specific for a sequence-variable target region set and/or target-binding probes specific for an epigenetic target region set. In some embodiments, the capture yield of the target-binding probes specific for the sequence-variable target region set is higher (e.g., at least 2-fold higher) than the capture yield of the target-binding probes specific for the epigenetic target region set. In some embodiments, the collection of capture probes is configured to have a capture yield specific for the sequence-variable target region set higher (e.g., at least 2-fold higher) than its capture yield specific for the epigenetic target region set.

In some embodiments, the capture yield of the target-binding probes specific for the sequence-variable target region set is at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold higher than the capture yield of the target-binding probes specific for the epigenetic target region set. In some embodiments, the capture yield of the target-binding probes specific for the sequence-variable target region set is 1.25- to 1.5-, 1.5- to 1.75-, 1.75- to 2-, 2- to 2.25-, 2.25- to 2.5-, 2.5- to 2.75-, 2.75- to 3-, 3- to 3.5-, 3.5- to 4-, 4- to 4.5-, 4.5- to 5-, 5- to 5.5-, 5.5- to 6-, 6- to 7-, 7- to 8-, 8- to 9-, 9- to 10-, 10-to 11-, 11- to 12-, 13- to 14-, or 14- to 15-fold higher than the capture yield of the target-binding probes specific for the epigenetic target region set.

In some embodiments, the collection of capture probes is configured to have a capture yield specific for the sequence-variable target region set at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold higher than its capture yield for the epigenetic target region set. In some embodiments, the collection of capture probes is configured to have a capture yield specific for the sequence-variable target region set is 1.25-to 1.5-, 1.5- to 1.75-, 1.75- to 2-, 2- to 2.25-, 2.25- to 2.5-, 2.5- to 2.75-, 2.75- to 3-, 3- to 3.5-, 3.5- to 4-, 4- to 4.5-, 4.5- to 5-, 5- to 5.5-, 5.5- to 6-, 6- to 7-, 7- to 8-, 8- to 9-, 9- to 10-, 10- to 11-, 11- to 12-, 13- to 14-, or 14- to 15-fold higher than its capture yield specific for the epigenetic target region set.

The collection of probes can be configured to provide higher capture yields for the sequence-variable target region set in various ways, including concentration, different lengths and/or chemistries (e.g., that affect affinity), and combinations thereof. Affinity can be modulated by adjusting probe length and/or including nucleotide modifications as discussed below.

In some embodiments, the capture probes specific for the sequence-variable target region set are present at a higher concentration than the capture probes specific for the epigenetic target region set. In some embodiments, concentration of the target-binding probes specific for the sequence-variable target region set is at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold higher than the concentration of the target-binding probes specific for the epigenetic target region set. In some embodiments, the concentration of the target-binding probes specific for the sequence-variable target region set is 1.25- to 1.5-, 1.5- to 1.75-, 1.75- to 2-, 2- to 2.25-, 2.25- to 2.5-, 2.5- to 2.75-, 2.75- to 3-, 3- to 3.5-, 3.5- to 4-, 4- to 4.5-, 4.5- to 5-, 5- to 5.5-, 5.5- to 6-, 6- to 7-, 7- to 8-, 8- to 9-, 9- to 10-, 10-to 11-, 11- to 12-, 13- to 14-, or 14- to 15-fold higher than the concentration of the target-binding probes specific for the epigenetic target region set. In such embodiments, concentration may refer to the average mass per volume concentration of individual probes in each set.

In some embodiments, the capture probes specific for the sequence-variable target region set have a higher affinity for their targets than the capture probes specific for the epigenetic target region set. Affinity can be modulated in any way known to those skilled in the art, including by using different probe chemistries. For example, certain nucleotide modifications, such as cytosine 5-methylation (in certain sequence contexts), modifications that provide a heteroatom at the 2′ sugar position, and LNA nucleotides, can increase stability of double-stranded nucleic acids, indicating that oligonucleotides with such modifications have relatively higher affinity for their complementary sequences. See, e.g., Severin et al., Nucleic Acids Res. 39: 8740-8751 (2011); Freier et al., Nucleic Acids Res. 25: 4429-4443 (1997); U.S. Pat. No. 9,738,894. Also, longer sequence lengths will generally provide increased affinity. Other nucleotide modifications, such as the substitution of the nucleobase hypoxanthine for guanine, reduce affinity by reducing the amount of hydrogen bonding between the oligonucleotide and its complementary sequence. In some embodiments, the capture probes specific for the sequence-variable target region set have modifications that increase their affinity for their targets. In some embodiments, alternatively or additionally, the capture probes specific for the epigenetic target region set have modifications that decrease their affinity for their targets. In some embodiments, the capture probes specific for the sequence-variable target region set have longer average lengths and/or higher average melting temperatures than the capture probes specific for the epigenetic target region set. These embodiments may be combined with each other and/or with differences in concentration as discussed above to achieve a desired fold difference in capture yield, such as any fold difference or range thereof described above.

In some embodiments, the capture probes comprise a capture moiety. The capture moiety may be any of the capture moieties described herein, e.g., biotin. In some embodiments, the capture probes are linked to a solid support, e.g., covalently or non-covalently such as through the interaction of a binding pair of capture moieties. In some embodiments, the solid support is a bead, such as a magnetic bead.

In some embodiments, the capture probes specific for the sequence-variable target region set and/or the capture probes specific for the epigenetic target region set are a capture probe set as discussed above, e.g., probes comprising capture moieties and sequences selected to tile across a panel of regions, such as genes.

In some embodiments, the capture probes are provided in a single composition. The single composition may be a solution (liquid or frozen). Alternatively, it may be a lyophilizate.

Alternatively, the capture probes may be provided as a plurality of compositions, e.g., comprising a first composition comprising probes specific for the epigenetic target region set and a second composition comprising probes specific for the sequence-variable target region set.

These probes may be mixed in appropriate proportions to provide a combined probe composition with any of the foregoing fold differences in concentration and/or capture yield. Alternatively, they may be used in separate capture procedures (e.g., with aliquots of a sample or sequentially with the same sample) to provide first and second compositions comprising captured epigenetic target regions and sequence-variable target regions, respectively.

### 1. Probes Specific for Epigenetic Target Regions

The probes for the epigenetic target region set may comprise probes specific for one or more types of target regions likely to differentiate DNA from neoplastic (e.g., tumor or cancer) cells from healthy cells, e.g., non-neoplastic circulating cells. Exemplary types of such regions are discussed in detail herein, e.g., in the sections above concerning captured sets. The probes for the epigenetic target region set may also comprise probes for one or more control regions, e.g., as described herein.

In some embodiments, the probes for the epigenetic target region set have a footprint of at least 100 kbp, e.g., at least 200 kbp, at least 300 kbp, or at least 400 kbp. In some embodiments, the epigenetic target region set has a footprint in the range of 100-20 Mbp, e.g., 100-200 kbp, 200-300 kbp, 300-400 kbp, 400-500 kbp, 500-600 kbp, 600-700 kbp, 700-800 kbp, 800-900 kbp, 900-1,000 kbp, 1-1.5 Mbp, 1.5-2 Mbp, 2-3 Mbp, 3-4 Mbp, 4-5 Mbp, 5-6 Mbp, 6-7 Mbp, 7-8 Mbp, 8-9 Mbp, 9-10 Mbp, or 10-20 Mbp. In some embodiments, the epigenetic target region set has a footprint of at least 20 Mbp.

a. Hypermethylation Variable Target Regions

In some embodiments, the probes for the epigenetic target region set comprise probes specific for one or more hypermethylation variable target regions. Hypermethylation variable target regions may also be referred to herein as hypermethylated DMRs (differentially methylated regions). The hypermethylation variable target regions may be any of those set forth above. For example, in some embodiments, the probes specific for hypermethylation variable target regions comprise probes specific for a plurality of loci listed in Table 1, e.g., at least 10%, 20%, 30%, 40%, 50%, 60%, 70%, 80%, 90%, or 100% of the loci listed in Table 1. In some embodiments, the probes specific for hypermethylation variable target regions comprise probes specific for a plurality of loci listed in Table 2, e.g., at least 10%, 20%, 30%, 40%, 50%, 60%, 70%, 80%, 90%, or 100% of the loci listed in Table 2. In some embodiments, the probes specific for hypermethylation variable target regions comprise probes specific for a plurality of loci listed in Table 1 or Table 2, e.g., at least 10%, 20%, 30%, 40%, 50%, 60%, 70%, 80%, 90%, or 100% of the loci listed in Table 1 or Table 2. In some embodiments, for each locus included as a target region, there may be one or more probes with a hybridization site that binds between the transcription start site and the stop codon (the last stop codon for genes that are alternatively spliced) of the gene. In some embodiments, the one or more probes bind within 300 bp of the listed position, e.g., within 200 or 100 bp. In some embodiments, a probe has a hybridization site overlapping the position listed above. In some embodiments, the probes specific for the hypermethylation target regions include probes specific for one, two, three, four, or five subsets of hypermethylation target regions that collectively show hypermethylation in one, two, three, four, or five of breast, colon, kidney, liver, and lung cancers.

b. Hypomethylation Variable Target Regions

In some embodiments, the probes for the epigenetic target region set comprise probes specific for one or more hypomethylation variable target regions. Hypomethylation variable target regions may also be referred to herein as hypomethylated DMRs (differentially methylated regions). The hypomethylation variable target regions may be any of those set forth above. For example, the probes specific for one or more hypomethylation variable target regions may include probes for regions such as repeated elements, e.g., LINE1 elements, Alu elements, centromeric tandem repeats, pericentromeric tandem repeats, and satellite DNA, and intergenic regions that are ordinarily methylated in healthy cells may show reduced methylation in tumor cells.

In some embodiments, probes specific for hypomethylation variable target regions include probes specific for repeated elements and/or intergenic regions. In some embodiments, probes specific for repeated elements include probes specific for one, two, three, four, or five of LINE1 elements, Alu elements, centromeric tandem repeats, pericentromeric tandem repeats, and/or satellite DNA.

Exemplary probes specific for genomic regions that show cancer-associated hypomethylation include probes specific for nucleotides 8403565-8953708 and/or 151104701-151106035 of human chromosome 1. In some embodiments, the probes specific for hypomethylation variable target regions include probes specific for regions overlapping or comprising nucleotides 8403565-8953708 and/or 151104701-151106035 of human chromosome 1.

c. CTCF Binding Regions

In some embodiments, the probes for the epigenetic target region set include probes specific for CTCF binding regions. In some embodiments, the probes specific for CTCF binding regions comprise probes specific for at least 10, 20, 50, 100, 200, or 500 CTCF binding regions, or 10-20, 20-50, 50-100, 100-200, 200-500, or 500-1000 CTCF binding regions, e.g., such as CTCF binding regions described above or in one or more of CTCFBSDB or the Cuddapah et al., Martin et al., or Rhee et al. articles cited above. In some embodiments, the probes for the epigenetic target region set comprise at least 100 bp, at least 200 bp at least 300 bp, at least 400 bp, at least 500 bp, at least 750 bp, or at least 1000 bp upstream and downstream regions of the CTCF binding sites.

d. Transcription Start Sites

In some embodiments, the probes for the epigenetic target region set include probes specific for transcriptional start sites. In some embodiments, the probes specific for transcriptional start sites comprise probes specific for at least 10, 20, 50, 100, 200, or 500 transcriptional start sites, or 10-20, 20-50, 50-100, 100-200, 200-500, or 500-1000 transcriptional start sites, e.g., such as transcriptional start sites listed in DBTSS. In some embodiments, the probes for the epigenetic target region set comprise probes for sequences at least 100 bp, at least 200 bp, at least 300 bp, at least 400 bp, at least 500 bp, at least 750 bp, or at least 1000 bp upstream and downstream of the transcriptional start sites.

e. Focal Amplifications

As noted above, although focal amplifications are somatic mutations, they can be detected by sequencing based on read frequency in a manner analogous to approaches for detecting certain epigenetic changes such as changes in methylation. As such, regions that may show focal amplifications in cancer can be included in the epigenetic target region set, as discussed above. In some embodiments, the probes specific for the epigenetic target region set include probes specific for focal amplifications. In some embodiments, the probes specific for focal amplifications include probes specific for one or more of AR, BRAF, CCND1, CCND2, CCNE1, CDK4, CDK6, EGFR, ERBB2, FGFR1, FGFR2, KIT, KRAS, MET, MYC, PDGFRA, PIK3CA, and RAF1. For example, in some embodiments, the probes specific for focal amplifications include probes specific for one or more of at least 2, 3, 4, 5, 6, 7, 8, 9, 10, 11, 12, 13, 14, 15, 16, 17, or 18 of the foregoing targets.

f. Control Regions

It can be useful to include control regions to facilitate data validation. In some embodiments, the probes specific for the epigenetic target region set include probes specific for control methylated regions that are expected to be methylated in essentially all samples. In some embodiments, the probes specific for the epigenetic target region set include probes specific for control hypomethylated regions that are expected to be hypomethylated in essentially all samples.

### 2. Probes Specific for Sequence-Variable Target Regions

The probes for the sequence-variable target region set may comprise probes specific for a plurality of regions known to undergo somatic mutations in cancer. The probes may be specific for any sequence-variable target region set described herein. Exemplary sequence-variable target region sets are discussed in detail herein, e.g., in the sections above concerning captured sets.

In some embodiments, the sequence-variable target region probe set has a footprint of at least 0.5 kb, e.g., at least 1 kb, at least 2 kb, at least 5 kb, at least 10 kb, at least 20 kb, at least 30 kb, or at least 40 kb. In some embodiments, the epigenetic target region probe set has a footprint in the range of 0.5-100 kb, e.g., 0.5-2 kb, 2-10 kb, 10-20 kb, 20-30 kb, 30-40 kb, 40-50 kb, 50-60 kb, 60-70 kb, 70-80 kb, 80-90 kb, and 90-100 kb. In some embodiments, the sequence-variable target region probe set has a footprint of at least 50 kbp, e.g., at least 100 kbp, at least 200 kbp, at least 300 kbp, or at least 400 kbp. In some embodiments, the sequence-variable target region probe set has a footprint in the range of 100-2000 kbp, e.g., 100-200 kbp, 200-300 kbp, 300-400 kbp, 400-500 kbp, 500-600 kbp, 600-700 kbp, 700-800 kbp, 800-900 kbp, 900-1,000 kbp, 1-1.5 Mbp or 1.5-2 Mbp. In some embodiments, the sequence-variable target region set has a footprint of at least 2 Mbp.

In some embodiments, probes specific for the sequence-variable target region set comprise probes specific for at least a portion of at least 5, at least 10, at least 15, at least 20, at least 25, at least 30, at least 35, at least 40, at least 45, at least 50, at least 55, at least 60, at least 65, or at 70 of the genes of Table 3. In some embodiments, probes specific for the sequence-variable target region set comprise probes specific for the at least 5, at least 10, at least 15, at least 20, at least 25, at least 30, at least 35, at least 40, at least 45, at least 50, at least 55, at least 60, at least 65, or 70 of the SNVs of Table 3. In some embodiments, probes specific for the sequence-variable target region set comprise probes specific for at least 1, at least 2, at least 3, at least 4, at least 5, or 6 of the fusions of Table 3. In some embodiments, probes specific for the sequence-variable target region set comprise probes specific for at least a portion of at least 1, at least 2, or 3 of the indels of Table 3. In some embodiments, probes specific for the sequence-variable target region set comprise probes specific for at least a portion of at least 5, at least 10, at least 15, at least 20, at least 25, at least 30, at least 35, at least 40, at least 45, at least 50, at least 55, at least 60, at least 65, at least 70, or 73 of the genes of Table 4. In some embodiments, probes specific for the sequence-variable target region set comprise probes specific for at least 5, at least 10, at least 15, at least 20, at least 25, at least 30, at least 35, at least 40, at least 45, at least 50, at least 55, at least 60, at least 65, at least 70, or 73 of the SNVs of Table 4. In some embodiments, probes specific for the sequence-variable target region set comprise probes specific for at least 1, at least 2, at least 3, at least 4, at least 5, or 6 of the fusions of Table 4. In some embodiments, probes specific for the sequence-variable target region set comprise probes specific for at least a portion of at least 1, at least 2, at least 3, at least 4, at least 5, at least 6, at least 7, at least 8, at least 9, at least 10, at least 11, at least 12, at least 13, at least 14, at least 15, at least 16, at least 17, or 18 of the indels of Table 4. In some embodiments, probes specific for the sequence-variable target region set comprise probes specific for at least a portion of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 of the genes of Table 5.

TABLE 3

| Point Mutations (SNVs) | | | | | | Fusions |
|---|---|---|---|---|---|---|
| AKT1 | ALK | APC | AR | ARAF | ARID1A | ALK |
| ATM | BRAF | BRCA1 | BRCA2 | CCND1 | CCND2 | FGFR2 |
| CCNE1 | CDH1 | CDK4 | CDK6 | CDKN2A | CDKN2B | FGFR3 |
| CTNNB1 | EGFR | ERBB2 | ESR1 | EZH2 | FBXW7 | NTRK1 |
| FGFR1 | FGFR2 | FGFR3 | GATA3 | GNA11 | GNAQ | RET |
| GNAS | HNF1A | HRAS | IDH1 | IDH2 | JAK2 | ROS1 |
| JAK3 | KIT | KRAS | MAP2K1 | MAP2K2 | MET | |
| MLH1 | MPL | MYC | NF1 | NFE2L2 | NOTCH1 | |
| NPM1 | NRAS | NTRK1 | PDGFRA | PIK3CA | PTEN | |
| PTPN11 | RAF1 | RB1 | RET | RHEB | RHOA | |
| RIT1 | ROS1 | SMAD4 | SMO | SRC | STK11 | |
| TERT | TP53 | TSC1 | VHL | | | |

TABLE 4

| Point Mutations (SNVs) | | | | | | Fusions |
|---|---|---|---|---|---|---|
| AKT1 | ALK | APC | AR | ARAF | ARID1A | ALK |
| ATM | BRAF | BRCA1 | BRCA2 | CCND1 | CCND2 | FGFR2 |
| CCNE1 | CDH1 | CDK4 | CDK6 | CDKN2A | DDR2 | FGFR3 |
| CTNNB1 | EGFR | ERBB2 | ESR1 | EZH2 | FBXW7 | NTRK1 |
| FGFR1 | FGFR2 | FGFR3 | GATA3 | GNA11 | GNAQ | RET |
| GNAS | HNF1A | HRAS | IDH1 | IDH2 | JAK2 | ROS1 |
| JAK3 | KIT | KRAS | MAP2K1 | MAP2K2 | MET | |
| MLH1 | MPL | MYC | NF1 | NFE2L2 | NOTCH1 | |
| NPM1 | NRAS | NTRK1 | PDGFRA | PIK3CA | PTEN | |
| PTPN11 | RAF1 | RB1 | RET | RHEB | RHOA | |
| RIT1 | ROS1 | SMAD4 | SMO | MAPK1 | STK11 | |
| TERT | TP53 | TSC1 | VHL | MAPK3 | MTOR | |
| NTRK3 | | | | | | |

TABLE 5

| | | Start | Stop | Length | Exons | |
|---|---|---|---|---|---|---|
| Gene | Chromosome | Position | Position | (bp) | Covered | Critical Feature |
| ALK | chr2 | 29446405 | 29446655 | 250 | intron 19 | Fusion |
| ALK | chr2 | 29446062 | 29446197 | 135 | intron 20 | Fusion |
| ALK | chr2 | 29446198 | 29446404 | 206 | 20 | Fusion |
| ALK | chr2 | 29447353 | 29447473 | 120 | intron 19 | Fusion |
| ALK | chr2 | 29447614 | 29448316 | 702 | intron 19 | Fusion |
| ALK | chr2 | 29448317 | 29448441 | 124 | 19 | Fusion |
| ALK | chr2 | 29449366 | 29449777 | 411 | intron 18 | Fusion |
| ALK | chr2 | 29449778 | 29449950 | 172 | 18 | Fusion |
| BRAF | chr7 | 140453064 | 140453203 | 139 | 15 | BRAF V600 |
| CTNNB1 | chr3 | 41266007 | 41266254 | 247 | 3 | S37 |
| EGFR | chr7 | 55240528 | 55240827 | 299 | 18 and 19 | G719 and deletions |
| EGFR | chr7 | 55241603 | 55241746 | 143 | 20 | Insertions/T790M |
| EGFR | chr7 | 55242404 | 55242523 | 119 | 21 | L858R |
| ERBB2 | chr17 | 37880952 | 37881174 | 222 | 20 | Insertions |
| ESR1 | chr6 | 152419857 | 152420111 | 254 | 10 | V534, P535, L536, |
| | | | | | | Y537, D538 |
| FGFR2 | chr10 | 123279482 | 123279693 | 211 | 6 | S252 |
| GATA3 | chr10 | 8111426 | 8111571 | 145 | 5 | SS/Indels |
| GATA3 | chr10 | 8115692 | 8116002 | 310 | 6 | SS/Indels |
| GNAS | chr20 | 57484395 | 57484488 | 93 | 8 | R844 |
| IDH1 | chr2 | 209113083 | 209113394 | 311 | 4 | R132 |
| IDH2 | chr15 | 90631809 | 90631989 | 180 | 4 | R140, R172 |
| KIT | chr4 | 55524171 | 55524258 | 87 | 1 | |
| KIT | chr4 | 55561667 | 55561957 | 290 | 2 | |
| KIT | chr4 | 55564439 | 55564741 | 302 | 3 | |
| KIT | chr4 | 55565785 | 55565942 | 157 | 4 | |
| KIT | chr4 | 55569879 | 55570068 | 189 | 5 | |
| KIT | chr4 | 55573253 | 55573463 | 210 | 6 | |
| KIT | chr4 | 55575579 | 55575719 | 140 | 7 | |
| KIT | chr4 | 55589739 | 55589874 | 135 | 8 | |
| KIT | chr4 | 55592012 | 55592226 | 214 | 9 | |
| KIT | chr4 | 55593373 | 55593718 | 345 | 10 and 11 | 557, 559, 560, 576 |
| KIT | chr4 | 55593978 | 55594297 | 319 | 12 and 13 | V654 |
| KIT | chr4 | 55595490 | 55595661 | 171 | 14 | T670, S709 |
| KIT | chr4 | 55597483 | 55597595 | 112 | 15 | D716 |
| KIT | chr4 | 55598026 | 55598174 | 148 | 16 | L783 |
| KIT | chr4 | 55599225 | 55599368 | 143 | 17 | C809, R815, D816, |
| | | | | | | L818, D820, S821F, |
| | | | | | | N822, Y823 |
| KIT | chr4 | 55602653 | 55602785 | 132 | 18 | A829P |
| KIT | chr4 | 55602876 | 55602996 | 120 | 19 | |
| KIT | chr4 | 55603330 | 55603456 | 126 | 20 | |
| KIT | chr4 | 55604584 | 55604733 | 149 | 21 | |
| KRAS | chr12 | 25378537 | 25378717 | 180 | 4 | A146 |
| KRAS | chr12 | 25380157 | 25380356 | 199 | 3 | Q61 |
| KRAS | chr12 | 25398197 | 25398328 | 131 | 2 | G12/G13 |
| MET | chr7 | 116411535 | 116412255 | 720 | 13, 14, | MET exon 14 SS |
| | | | | | intron 13, | |
| | | | | | intron 14 | |
| NRAS | chr1 | 115256410 | 115256609 | 199 | 3 | Q61 |
| NRAS | chr1 | 115258660 | 115258791 | 131 | 2 | G12/G13 |
| PIK3CA | chr3 | 178935987 | 178936132 | 145 | 10 | E545K |
| PIK3CA | chr3 | 178951871 | 178952162 | 291 | 21 | H1047R |
| PTEN | chr10 | 89692759 | 89693018 | 259 | 5 | R130 |
| SMAD4 | chr18 | 48604616 | 48604849 | 233 | 12 | D537 |
| TERT | chr5 | 1294841 | 1295512 | 671 | promoter | chr5: 1295228 |
| TP53 | chr17 | 7573916 | 7574043 | 127 | 11 | Q331, R337, R342 |
| TP53 | chr17 | 7577008 | 7577165 | 157 | 8 | R273 |
| TP53 | chr17 | 7577488 | 7577618 | 130 | 7 | R248 |
| TP53 | chr17 | 7578127 | 7578299 | 172 | 6 | R213/Y220 |
| TP53 | chr17 | 7578360 | 7578564 | 204 | 5 | R175/Deletions |
| TP53 | chr17 | 7579301 | 7579600 | 299 | 4 | |
| | | | | 12574 | | |
| | | | | (total | | |
| | | | | target | | |
| | | | | region) | | |
| | | | | 16330 | | |
| | | | | (total | | |
| | | | | probe | | |
| | | | | coverage) | | |

In some embodiments, the probes specific for the sequence-variable target region set comprise probes specific for target regions from at least 10, 20, 30, or 35 cancer-related genes, such as AKT1, ALK, BRAF, CCND1, CDK2A, CTNNB1, EGFR, ERBB2, ESR1, FGFR1, FGFR2, FGFR3, FOXL2, GATA3, GNA11, GNAQ, GNAS, HRAS, IDH1, IDH2, KIT, KRAS, MED12, MET, MYC, NFE2L2, NRAS, PDGFRA, PIK3CA, PPP2R1A, PTEN, RET, STK11, TP53, and U2AF1.

### M. Computer Systems

Methods of the present disclosure can be implemented using, or with the aid of, computer systems. FIG. 2 shows a computer system 201 that is programmed or otherwise configured to implement the methods of the present disclosure. The computer system 201 can regulate various aspects sample preparation, sequencing, and/or analysis. In some examples, the computer system 201 is configured to perform sample preparation and sample analysis, including nucleic acid sequencing, e.g., according to any of the methods disclosed herein.

The computer system 201 includes a central processing unit (CPU, also “processor” and “computer processor” herein) 205, which can be a single core or multi core processor, or a plurality of processors for parallel processing. The computer system 201 also includes memory or memory location 210 (e.g., random-access memory, read-only memory, flash memory), electronic storage unit 215 (e.g., hard disk), communication interface 220 (e.g., network adapter) for communicating with one or more other systems, and peripheral devices 225, such as cache, other memory, data storage, and/or electronic display adapters. The memory 210, storage unit 215, interface 220, and peripheral devices 225 are in communication with the CPU 205 through a communication network or bus (solid lines), such as a motherboard. The storage unit 215 can be a data storage unit (or data repository) for storing data. The computer system 201 can be operatively coupled to a computer network 230 with the aid of the communication interface 220. The computer network 230 can be the Internet, an internet and/or extranet, or an intranet and/or extranet that is in communication with the Internet. The computer network 230 in some cases is a telecommunication and/or data network. The computer network 230 can include one or more computer servers, which can enable distributed computing, such as cloud computing. The computer network 230, in some cases with the aid of the computer system 0, can implement a peer-to-peer network, which may enable devices coupled to the computer system 201 to behave as a client or a server.

The CPU 205 can execute a sequence of machine-readable instructions, which can be embodied in a program or software. The instructions may be stored in a memory location, such as the memory 210. Examples of operations performed by the CPU 205 can include fetch, decode, execute, and writeback.

The storage unit 215 can store files, such as drivers, libraries, and saved programs. The storage unit 215 can store programs generated by users and recorded sessions, as well as output(s) associated with the programs. The storage unit 215 can store user data, e.g., user preferences and user programs. The computer system 201 in some cases can include one or more additional data storage units that are external to the computer system 201, such as located on a remote server that is in communication with the computer system 201 through an intranet or the Internet. Data may be transferred from one location to another using, for example, a communication network or physical data transfer (e.g., using a hard drive, thumb drive, or other data storage mechanism).

The computer system 201 can communicate with one or more remote computer systems through the network 230. For embodiment, the computer system 201 can communicate with a remote computer system of a user (e.g., operator). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 201 via the network 230.

Methods as described herein can be implemented by way of machine (e.g., computer processor) executable code stored on an electronic storage location of the computer system 201, such as, for example, on the memory 210 or electronic storage unit 215. The machine executable or machine-readable code can be provided in the form of software. During use, the code can be executed by the processor 205. In some cases, the code can be retrieved from the storage unit 215 and stored on the memory 210 for ready access by the processor 205. In some situations, the electronic storage unit 215 can be precluded, and machine-executable instructions are stored on memory 210.

In an aspect, the present disclosure provides a non-transitory computer-readable medium comprising computer-executable instructions which, when executed by at least one electronic processor, perform at least a portion of a method comprising: (a) performing a sequencing by synthesis reaction on a converted DNA molecule with a sequencing by synthesis instrument, the converted DNA molecule comprising: ligated adapters; a converted region comprising one or more nucleobases that have been converted by a conversion procedure; and a resistant region comprising one or more nucleobases that are resistant to the conversion procedure; wherein the sequencing by synthesis reaction comprises extending a sequencing primer that binds to the converted DNA molecule upstream of the converted region and the resistant region; (b) calibrating one or more base calling metrics of the sequencing by synthesis instrument based at least in part on data from the resistant region, thereby providing one or more calibrated base calling metrics; and (c) calling at least a portion of nucleobases in the converted region using the one or more calibrated base calling metrics. The code can be pre-compiled and configured for use with a machine have a processer adapted to execute the code or can be compiled during runtime. The code can be supplied in a programming language that can be selected to enable the code to execute in a pre-compiled or as-compiled fashion.

Aspects of the systems and methods provided herein, such as the computer system 201, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming.

All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as those used across physical interfaces between local devices, through wired and optical landline networks, and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine-readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards, paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 201 can include or be in communication with an electronic display that comprises a user interface (UI) for providing, for example, one or more results of sample analysis. Examples of UIs include, without limitation, a graphical user interface (GUI) and web-based user interface.

Additional details relating to computer systems and networks, databases, and computer program products are also provided in, for example, Peterson, Computer Networks: A Systems Approach, Morgan Kaufmann, 5th Ed. (2011), Kurose, Computer Networking: A Top-Down Approach, Pearson, 7^{th} Ed. (2016), Elmasri, Fundamentals of Database Systems, Addison Wesley, 6th Ed. (2010), Coronel, Database Systems: Design, Implementation, & Management, Cengage Learning, 11^{th} Ed. (2014), Tucker, Programming Languages, McGraw-Hill Science/Engineering/Math, 2nd Ed. (2006), and Rhoton, Cloud Computing Architected: Solution Design Handbook, Recursive Press (2011), each of which is hereby incorporated by reference in its entirety.

### N. Applications

### 1. Cancer and Other Diseases; Cell Type Quantification

The present methods can be used to diagnose the presence of a condition, e.g., cancer or precancer, in a subject, to characterize a condition (such as to determine a cancer stage or determining heterogeneity of a cancer), to monitor a subject's response to receiving a treatment for a condition (such as a response to a chemotherapeutic or immunotherapeutic), assess prognosis of a subject (such as to predict a survival outcome in a subject having a cancer), to determine a subject's risk of developing a condition, to predict a subsequent course of a condition in a subject, to determine metastasis or recurrence of a cancer in a subject (or a risk of cancer metastasis or recurrence), and/or to monitor a subject's health as part of a preventative health monitoring program (such as to determine whether and/or when a subject is in need of further diagnostic screening). The present disclosure can also be useful in determining the efficacy of a particular treatment option. Successful treatment options may increase the amount of copy number variation, rare mutations, and/or cancer-related epigenetic signatures (such as hypermethylated regions or hypomethylated regions) detected in a subject's blood (such as in DNA isolated from a buffy coat sample or any other sample comprising cells, such as a blood sample (e.g., a whole blood sample, a leukapheresis sample, or a PBMC sample) from the subject) if the treatment is successful as more cancer cells may die and shed DNA, or, e.g., if a successful treatment results in an increase or decrease in the quantity of a specific immune cell type in the blood and an unsuccessful treatment results in no change. In other examples, this may not occur. In another example, certain treatment options may be correlated with genetic profiles of cancers over time. This correlation may be useful in selecting a therapy for a subject.

In some embodiments, the present methods are used for screening for a cancer, such as a metastasis, or in a method for screening cancer, such as in a method of detecting the presence or absence of a metastasis. For example, the sample can be a sample from a subject who has not been previously diagnosed with cancer. In some embodiments, one or more, such as 1, 2, 3, 4, 5, 6, 7, 8, 9, 10, or more samples are collected from a subject as described herein, such as before and/or after the subject is diagnosed with a cancer. In some embodiments, the subject may or may not have cancer. In some embodiments, the subject may or may not have an early-stage cancer. In some embodiments, the subject has one or more risk factors for cancer, such as tobacco use (e.g., smoking), being overweight or obese, having a high body mass index (BMI), being of advanced age, poor nutrition, high alcohol consumption, or a family history of cancer.

In some embodiments, the subject has used tobacco, e.g., for at least 1, 5, 10, or 15 years. In some embodiments, the subject has a high BMI, e.g., a BMI of 25 or greater, 26 or greater, 27 or greater, 28 or greater, 29 or greater, or 30 or greater. In some embodiments, the subject is at least 40, 45, 50, 55, 60, 65, 70, 75, or 80 years old. In some embodiments, the subject has poor nutrition, e.g., high consumption of one or more of red meat and/or processed meat, trans fat, saturated fat, and refined sugars, and/or low consumption of fruits and vegetables, complex carbohydrates, and/or unsaturated fats. High and low consumption can be defined, e.g., as exceeding or falling below, respectively, recommendations in Dietary Guidelines for Americans 2020-2025, available at dietaryguidelines.gov/sites/default/files/2021-03/Dietary_Guidelines_for_Americans-2020-2025.pdf In some embodiments, the subject has high alcohol consumption, e.g., at least three, four, or five drinks per day on average (where a drink is about one ounce or 30 mL of 80-proof hard liquor or the equivalent). In some embodiments, the subject has a family history of cancer, e.g., at least one, two, or three blood relatives were previously diagnosed with cancer. In some embodiments, the relatives are at least third-degree relatives (e.g., great-grandparent, great aunt or uncle, first cousin), at least second-degree relatives (e.g., grandparent, aunt or uncle, or half-sibling), or first-degree relatives (e.g., parent or full sibling).

Typically, the disease under consideration is a type of cancer. Non-limiting examples of such cancers include biliary tract cancer, bladder cancer, transitional cell carcinoma, urothelial carcinoma, brain cancer, gliomas, astrocytomas, breast cancer, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, Wilms tumor, lymphocytic cancer, leukemia, acute lymphocytic leukemia (ALL), acute myeloid leukemia (AML), chronic lymphocytic leukemia (CLL), chronic myeloid leukemia (CML), chronic myelomonocytic leukemia (CMML), liver cancer, liver carcinoma, hepatoma, hepatocellular carcinoma, cholangiocarcinoma, hepatoblastoma, Lung cancer, non-small cell lung cancer (NSCLC), mesothelioma, lymphoma, B-cell lymphomas, non-Hodgkin lymphoma, diffuse large B-cell lymphoma, Mantle cell lymphoma, T cell lymphomas, non-Hodgkin lymphoma, precursor T-lymphoblastic lymphoma/leukemia, peripheral T cell lymphomas, myeloma, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas, Prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, or uterine sarcoma.

In some embodiments, the cancer is a type of cancer that is not a hematological cancer, e.g., a solid tumor cancer such as a carcinoma, adenocarcinoma, or sarcoma. Type and/or stage of cancer can be detected from genetic variations including mutations, rare mutations, indels, rearrangements, copy number variations, transversions, translocations, recombinations, inversion, deletions, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, such as 5mC and 5mC profiles. Hence, the present methods can in some cases be used in combination with methods used to detect other genetic/epigenetic variations, e.g. in a method of detecting or characterizing a cancer or other methods described herein. In some embodiments, a method described herein comprises identifying the presence of target regions and/or DNA produced by a tumor (or neoplastic cells, or cancer cells) or by precancer cells. In some embodiments, a method described herein comprises determining the level of target regions and/or identifying the presence of DNA produced by a tumor (or neoplastic cells, or cancer cells) or by precancer cells. In some embodiments, determining the level of target regions comprises determining either an increased level or decreased level of target regions, wherein the increased or decreased level of target regions is determined by comparing the level of target regions with a threshold level/value.

The present methods can be used to diagnose, prognose, monitor or observe cancers, precancers, or other diseases. In some embodiments, the methods herein do not involve the diagnosing, prognosing or monitoring a fetus and as such are not directed to non-invasive prenatal testing. In other embodiments, these methodologies may be employed in a pregnant subject to diagnose, prognose, monitor or observe cancers or other diseases in an unborn subject whose DNA and other polynucleotides may co-circulate with maternal molecules. Non-limiting examples of other genetic-based diseases, disorders, or conditions that are optionally evaluated using the methods and systems disclosed herein include achondroplasia, alpha-1 antitrypsin deficiency, antiphospholipid syndrome, autism, autosomal dominant polycystic kidney disease, Charcot-Marie-Tooth (CMT), cri du chat, Crohn's disease, cystic fibrosis, Dercum disease, down syndrome, Duane syndrome, Duchenne muscular dystrophy, Factor V Leiden thrombophilia, familial hypercholesterolemia, familial mediterranean fever, fragile X syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency (scid), sickle cell disease, spinal muscular atrophy, Tay-Sachs, thalassemia, trimethylaminuria, Turner syndrome, velocardiofacial syndrome, WAGR syndrome, Wilson disease, or the like.

Genetic and/or epigenetic data can also be used for characterizing a specific form of cancer. Cancers are often heterogeneous in both composition and staging. Genetic profile data may allow characterization of specific sub-types of cancer that may be important in the diagnosis or treatment of that specific sub-type. This information may also provide a subject or practitioner clues regarding the prognosis of a specific type of cancer and allow either a subject or practitioner to adapt treatment options in accord with the progress of the disease. Some cancers can progress to become more aggressive and genetically unstable. Other cancers may remain benign, inactive or dormant. The system and methods of this disclosure may be useful in determining disease progression.

Further, the methods of the disclosure may be used to characterize the heterogeneity of an abnormal condition in a subject. Such methods can include, e.g., generating a genetic profile of extracellular polynucleotides (e.g., cfDNA) derived from the subject, e.g., wherein the genetic profile comprises a plurality of data resulting from copy number variation and rare mutation analyses. In some embodiments, an abnormal condition is cancer, e.g., as described herein. In some embodiments, the abnormal condition may be one resulting in a heterogeneous genomic population. In the example of cancer, some tumors are known to comprise tumor cells in different stages of the cancer. In other examples, heterogeneity may comprise multiple foci of disease, such as where one or more foci (such as one or more tumor foci) are the result of metastases that have spread from a primary site of a cancer. The tissue(s) of origin can be useful for identifying organs affected by the cancer, including the primary cancer and/or metastatic tumors.

The present methods can be used to generate or profile, fingerprint or set of data that is a summation of genetic information derived from different cells in a heterogeneous disease. This set of data may comprise copy number variation, epigenetic variation, and mutation analyses alone or in combination.

In some embodiments, the sample is obtained from a subject who was previously diagnosed with a cancer and received one or more previous cancer treatments. In some embodiments, the sample is obtained at one or more preselected time points following the one or more previous cancer treatments. In some embodiments, a method described herein comprises detecting a presence or absence of a nucleic acid originating or derived from a tumor cell at a preselected timepoint following a previous cancer treatment of a subject previously diagnosed with cancer. The method may further comprise determining a cancer recurrence score that is indicative of the presence or levels of DNA and/or RNA originating or derived from the tumor cell for the subject.

Where a cancer recurrence score is determined, it may further be used to determine a cancer recurrence status. The cancer recurrence status may be at risk for cancer recurrence, e.g., when the cancer recurrence score is above a predetermined threshold. The cancer recurrence status may be at low or lower risk for cancer recurrence, e.g., when the cancer recurrence score is above a predetermined threshold. In particular embodiments, a cancer recurrence score equal to the predetermined threshold may result in a cancer recurrence status of either at risk for cancer recurrence or at low or lower risk for cancer recurrence.

In some embodiments, a cancer recurrence score is compared with a predetermined cancer recurrence threshold, and the subject is classified as a candidate for a subsequent cancer treatment when the cancer recurrence score is above the cancer recurrence threshold or not a candidate for therapy when the cancer recurrence score is below the cancer recurrence threshold. In particular embodiments, a cancer recurrence score equal to the cancer recurrence threshold may result in classification as either a candidate for a subsequent cancer treatment or not a candidate for therapy.

Furthermore, in some embodiments, the one or more methods described in the present disclosure may be used to assist in the treatment of a type of cancer.

These methods provided herein provide a deeper understanding of the changes in DNA and proteins that cause cancer, allowing the identification of biomarkers and design of treatments that target these proteins. In some embodiments, the biomarker may include an epigenetic signature, such as a methylation state, methylation score and/or DNA fragmentation pattern/score. In some embodiments, the epigenetic signature can be determined for one or more regions that include, but not limited to, transcription start sites, promoter regions, CTCF binding regions and regulatory protein binding regions. In some embodiments, the epigenetic signature is determined for one or more regions that include, but not limited to, transcription start sites, promoter regions, intergenic regions and/or intronic regions that are associated with at least one or more genes listed in any one or more of Tables 1, 2, 3, 4, and 5. Such treatments may include small-molecule drugs or monoclonal antibodies. The methods may also improve biomarker testing in individuals suffering from disease and help determine if the individual is a candidate for a certain drug or combination of drugs based on the presence or absence of the biomarker. Additionally, the methods can improve identification of mutations that contribute to the development of resistance to targeted therapy. Consequently, the analysis techniques may reduce unnecessary or untimely therapeutic interventions, patient suffering, and patient mortality.

The present methods can also be used to quantify levels of different cell types, such as cancer cell types and/or immune cell types, including rare immune cell types, such as activated lymphocytes and myeloid cells at particular stages of differentiation. Such quantification can be based on the numbers of molecules corresponding to a given cell type in a sample. In some embodiments, quantities of each of a plurality of cell types, such as cancer cell types and/or immune cell types, are determined based on sequencing and analysis (such as determination of epigenetic and/or genomic signatures) of DNA isolated from at least one sample comprising cells (such as a buffy coat sample or another type of blood sample (e.g., a whole blood sample, a leukapheresis sample, or a PBMC sample) from a subject. For example, a plurality of immune cell types can include, but is not limited to, macrophages (including M1 macrophages and M2 macrophages), activated B cells (including regulatory B cells, memory B cells and plasma cells); T cell subsets, such as central memory T cells, naïve-like T cells, and activated T cells (including cytotoxic T cells, regulatory T cells (Tregs), CD4 effector memory T cells, CD4 central memory T cells, CD8 effector memory T cells, and CD8 central memory T cells); immature myeloid cells (including myeloid-derived suppressor cells (MDSCs), low-density neutrophils, immature neutrophils, and immature granulocytes); and natural killer (NK) cells. As disclosed herein, differences in levels and/or presence of particular genetic and/or epigenetic signatures in DNA isolated from blood samples from a subject can be used to quantify cell types, such as cancer cell types and/or immune cell types, within the sample.

Sequence information obtained in the present methods may comprise sequence reads of the nucleic acids generated by a nucleic acid sequencer. In some embodiments, the nucleic acid sequencer performs pyrosequencing, single-molecule sequencing, nanopore sequencing, semiconductor sequencing, sequencing-by-synthesis, 5-letter sequencing, 6-letter sequencing, sequencing-by-ligation or sequencing-by-hybridization on the nucleic acids to generate sequencing reads. In some embodiments, the method further comprises grouping the sequence reads into families of sequence reads, each family comprising sequence reads generated from a nucleic acid in the sample. In some embodiments, the methods comprise determining the likelihood that the subject from which the sample was obtained has cancer, precancer, an infection, transplant rejection, or another disease or disorder. In some embodiments, the disease or disorder is related to changes in proportions of types of immune cells. As discussed herein, comparisons of cell identities and/or cell quantities/proportions (such as cancer cell identities and/or quantities/proportions, and/or immune cell identities and/or quantities/proportions) between two or more samples collected from a subject at two different time points can allow for monitoring of one or more aspects of a condition in the subject over time, such as a response of the subject to a treatment, the severity of the condition (such as a cancer stage) in the subject, a recurrence of the condition (such as a cancer), and/or the subject's risk of developing the condition (such as a cancer).

The methods discussed herein may further comprise any compatible feature or features set forth elsewhere herein, including in the section regarding methods of determining a risk of cancer recurrence in a subject and/or classifying a subject as being a candidate for a subsequent cancer treatment.

2. Methods of Determining a Risk of Cancer Recurrence in a Test Subject and/or Classifying a Test Subject as being a Candidate for a Subsequent Cancer Treatment

In some embodiments, a method provided herein is a method of determining a risk of cancer recurrence in a subject. In some embodiments, a method provided herein is or comprises a method of detecting the presence of absence of a metastasis in a subject. In some embodiments, a method provided herein is or comprises a method of classifying a subject as being a candidate for a subsequent cancer treatment.

Any of such methods may comprise collecting nucleic acids (e.g., DNA originating or derived from a tumor cell) from the subject diagnosed with the cancer at one or more preselected timepoints following one or more previous cancer treatments to the subject. The subject may be any of the subjects described herein. The DNA may be DNA, such as cfDNA, from a blood sample (e.g., a whole blood sample). The DNA may comprise DNA obtained from a tissue sample.

Any of such methods may comprise enriching for a plurality of sets of target regions from DNA and/or RNA from the subject, wherein the plurality of target region sets comprise a sequence-variable target region set, and/or an epigenetic target region set, whereby a captured set of nucleic acid molecules is produced. The enriching step may be performed according to any of the embodiments described elsewhere herein.

In any of such methods, the previous cancer treatment may comprise surgery, administration of a therapeutic composition, and/or chemotherapy.

Any of such methods may comprise sequencing the enriched DNA molecules or enriched cDNA molecules generated from RNA, whereby a set of sequence information is produced. The enriched DNA molecules of a sequence-variable target region set may be sequenced to a greater depth of sequencing than the captured DNA molecules of the epigenetic target region set.

Any of such methods may comprise detecting a presence or absence of DNA originating or derived from a tumor cell at a preselected timepoint using the set of sequence information. The detection of the presence or absence of DNA, such as cfDNA, originating or derived from a tumor cell may be performed according to any of the embodiments thereof described elsewhere herein.

Methods of determining a risk of cancer recurrence in a subject may comprise determining a cancer recurrence score that is indicative of the presence or absence, or amount, of the DNA, such as cfDNA, originating or derived from the tumor cell for the subject. The cancer recurrence score may further be used to determine a cancer recurrence status. The cancer recurrence status may be at risk for cancer recurrence, e.g., when the cancer recurrence score is above a predetermined threshold. The cancer recurrence status may be at low or lower risk for cancer recurrence, e.g., when the cancer recurrence score is above a predetermined threshold. In particular embodiments, a cancer recurrence score equal to the predetermined threshold may result in a cancer recurrence status of either at risk for cancer recurrence or at low or lower risk for cancer recurrence.

Methods of detecting the presence or absence of metastasis in a subject may comprise comparing the presence or level of a tissue-specific cell material to the presence or level of the tissue-specific cell material obtained from the subject at a different time, a reference level of the tissue-specific cell material, or to a comparator cell material. Methods herein may comprise additional steps to determine whether a metastasis is present.

Methods of classifying a subject as being a candidate for a subsequent cancer treatment may comprise comparing the cancer recurrence score of the subject with a predetermined cancer recurrence threshold, thereby classifying the subject as a candidate for the subsequent cancer treatment when the cancer recurrence score is above the cancer recurrence threshold or not a candidate for therapy when the cancer recurrence score is below the cancer recurrence threshold.

In particular embodiments, a cancer recurrence score equal to the cancer recurrence threshold may result in classification as either a candidate for a subsequent cancer treatment or not a candidate for therapy. In some embodiments, the subsequent cancer treatment comprises chemotherapy or administration of a therapeutic composition.

Any of such methods may comprise determining a disease-free survival (DFS) period for the subject based on the cancer recurrence score; for example, the DFS period may be 1 year, 2 years, 3, years, 4 years, 5 years, or 10 years.

In some embodiments, the set of sequence information comprises sequence-variable target region sequences and determining the cancer recurrence score may comprise determining at least a first subscore indicative of SNVs, insertions/deletions, CNVs and/or fusions present in sequence-variable target region sequences.

In some embodiments, a number of mutations in the sequence-variable target regions chosen from 1, 2, 3, 4, or 5 is sufficient for the first subscore to result in a cancer recurrence score classified as positive for cancer recurrence. In some embodiments, the number of mutations is chosen from 1, 2, or 3.

In some embodiments, the set of sequence information comprises epigenetic target region sequences, and determining the cancer recurrence score comprises determining a second subscore indicative of the amount of molecules (obtained from the epigenetic target region sequences) that represent an epigenetic state different from DNA found in a corresponding sample from a healthy subject (e.g., DNA, such as cfDNA, from a blood sample (e.g., a whole blood sample), and/or DNA found in a tissue sample from a healthy subject where the tissue sample is of the same type of tissue as was obtained from the subject). These abnormal molecules (i.e., molecules with an epigenetic state different from DNA found in a corresponding sample from a healthy subject) may be consistent with epigenetic changes associated with cancer, e.g., methylation of hypermethylation variable target regions and/or perturbed fragmentation of fragmentation variable target regions, where “perturbed” means different from DNA found in a corresponding sample from a healthy subject.

In some embodiments, a proportion of molecules corresponding to the hypermethylation variable target region set and/or fragmentation variable target region set that indicate hypermethylation in the hypermethylation variable target region set and/or abnormal fragmentation in the fragmentation variable target region set greater than or equal to a value in the range of 0.001%-10% is sufficient for the second subscore to be classified as positive for cancer recurrence. The range may be 0.001%-1%, 0.005%-1%, 0.01%-5%, 0.01%-2%, or 0.01%-1%.

In some embodiments, any of such methods may comprise determining a fraction of tumor DNA from the fraction of molecules in the set of sequence information that indicate one or more features indicative of origination from a tumor cell. This may be done for molecules corresponding to some or all of the target regions, e.g., including one or both of hypermethylation variable target regions, hypomethylation variable target regions, and fragmentation variable target regions (hypermethylation of a hypermethylation variable target region and/or abnormal fragmentation of a fragmentation variable target region may be considered indicative of origination from a tumor cell). This may be done for molecules corresponding to sequence variable target regions, e.g., molecules comprising alterations consistent with cancer, such as SNVs, indels, CNVs, and/or fusions. The fraction of tumor DNA may be determined based on a combination of molecules corresponding to epigenetic target regions and molecules corresponding to sequence variable target regions.

Determination of a cancer recurrence score may be based at least in part on the fraction of tumor DNA, wherein a fraction of tumor DNA greater than a threshold in the range of 10⁻¹¹ to 1 or 10⁻¹⁰ to 1 is sufficient for the cancer recurrence score to be classified as positive for cancer recurrence. In some embodiments, a fraction of tumor DNA greater than or equal to a threshold in the range of 10⁻¹⁰ to 10⁻⁹, 10⁻⁹ to 10⁻⁸, 10⁻⁸ to 10⁻⁷, 10⁻⁷ to 10⁻⁶, 10⁻⁶ to 10⁻⁵, 10⁻⁵ to 10⁻⁴, 10⁻⁴ to 10⁻³, 10⁻³ to 10⁻², or 10⁻² to 10⁻¹ is sufficient for the cancer recurrence score to be classified as positive for cancer recurrence. In some embodiments, the fraction of tumor DNA greater than a threshold of at least 10⁻⁷ is sufficient for the cancer recurrence score to be classified as positive for cancer recurrence. A determination that a fraction of tumor DNA is greater than a threshold, such as a threshold corresponding to any of the foregoing embodiments, may be made based on a cumulative probability. For example, the sample was considered positive if the cumulative probability that the tumor fraction was greater than a threshold in any of the foregoing ranges exceeds a probability threshold of at least 0.5, 0.75, 0.9, 0.95, 0.98, 0.99, 0.995, or 0.999. In some embodiments, the probability threshold is at least 0.95, such as 0.99.

In some embodiments, the set of sequence information comprises sequence-variable target region sequences and epigenetic target region sequences, and determining the cancer recurrence score comprises determining a first subscore indicative of the amount of SNVs, insertions/deletions, CNVs and/or fusions present in sequence-variable target region sequences and a second subscore indicative of the amount of abnormal molecules in epigenetic target region sequences, and combining the first and second subscores to provide the cancer recurrence score. Where the subscores are combined, they may be combined by applying a threshold to each subscore independently in sequence-variable target regions, respectively, and greater than a predetermined fraction of abnormal molecules (i.e., molecules with an epigenetic state different from the DNA found in a corresponding sample from a healthy subject; e.g., tumor) in epigenetic target regions), or training a machine learning classifier to determine status based on a plurality of positive and negative training samples.

In some embodiments, a value for the combined score in the range of −4 to 2 or -3 to 1 is sufficient for the cancer recurrence score to be classified as positive for cancer recurrence.

In any embodiment where a cancer recurrence score is classified as positive for cancer recurrence, the cancer recurrence status of the subject may be at risk for cancer recurrence and/or the subject may be classified as a candidate for a subsequent cancer treatment.

In some embodiments, the cancer is any one of the types of cancer described elsewhere herein, e.g., colorectal cancer.

### 3. Methods of Monitoring a Cancer in a Subject Over Time; Sample Collection at Two or More Time Points

In some embodiments, the present methods can be used to monitor one or more aspects of a condition in a subject over time, such as a subject's response to receiving a treatment for a condition (such as a response to a chemotherapeutic or immunotherapeutic), the severity of the condition (such as a cancer stage) in the subject, a recurrence of the condition (such as a cancer), and/or the subject's risk of developing the condition (such as a cancer) and/or to monitor a subject's health as part of a preventative health monitoring program (such as to determine whether and/or when a subject is in need of further diagnostic screening). In some embodiments, monitoring comprises analysis of at least two samples collected from a subject at at least two different time points as described herein.

The methods according to the present disclosure can be useful in predicting a subject's response to a particular treatment option, such as over a period of time. As described elsewhere herein, successful treatment options may increase the amount of cancer associated DNA sequences detected in a subject's blood, such as if the treatment is successful as more cancers may die and shed DNA. In such examples, certain treatment options may be correlated with genetic profiles of cancers over time. This correlation may be useful in selecting a therapy.

As disclosed herein, in some embodiments, quantities of each of a plurality of cell types, such as cancer cell types and/or immune cell types, are determined based on sequencing and analysis (such as determination of epigenetic and/or genomic signatures) of DNA isolated from at least one sample comprising cells (such as a buffy coat sample or another type of blood sample (e.g., a whole blood sample, a leukapheresis sample, or a PBMC sample) from a subject.

In some embodiments, differences in levels and/or presence of particular genetic and/or epigenetic signatures in DNA isolated from blood samples from a subject can be used to quantify cell types, such as cancer cell types and/or immune cell types, within the sample. Thus, a comparison of the disclosed genetic and/or epigenetic signatures in DNA isolated from blood samples collected from a subject at two or more time points can be used to monitor changes in cell type quantities in the subject under different conditions (such as prior to and after a treatment), or over time (e.g., as part of a preventative health monitoring program).

The disclosed methods can include evaluating (such as quantifying) and/or interpreting cell types (such as cancer cell types and/or immune cell types) present in one or more samples comprising cells (such as a buffy coat sample or another type of blood sample (e.g., a whole blood sample, a leukapheresis sample, or a PBMC sample), a leukapheresis sample, or a PBMC sample) collected from a subject at one or more timepoints in comparison to a selected baseline value or reference standard (or a selected set of baseline values or reference standards). A baseline value or reference standard may be a quantity of cell types measured in one or more samples (such as an average quantity or range of quantities of cell types present in at least two samples) collected from the subject at one or more time points, such as prior to receiving a treatment, prior to diagnosis of a condition (such as a cancer), or as part of a preventative health monitoring program. A baseline value or reference standard may be a quantity of cell types measured in one or more samples (such as an average quantity or range of quantities of cell types present in at least two samples) collected at one or more timepoints from one or more subjects that do not have the condition (such as a healthy subject that does not have a cancer), one or more subjects that responded favorably to the treatment, or one or more subjects that have not received the treatment. In certain embodiments, the baseline value or reference standard utilized is a standard or profile derived from a single reference subject. In other embodiments, the baseline value or reference standard utilized is a standard or profile derived from averaged data from multiple reference subjects. The reference standard, in various embodiments, can be a single value, a mean, an average, a numerical mean or range of numerical means, a numerical pattern, or a graphical pattern created from the cell type quantity data derived from a single reference subject or from multiple reference subjects. Selection of the particular baseline values or reference standards, or selection of the one or more reference subjects, depends upon the use to which the methods described herein are to be put by, for example, a research scientist or a clinician (such as a physician).

In some embodiments, one or more samples comprising cells (such as a tissue sample or a blood sample (e.g., a whole blood sample, a buffy coat sample, a leukapheresis sample, or a PBMC sample)) may be collected from a subject at two or more timepoints, to assess changes in cell types (such as changes in quantities of cell types) between the two or more timepoints. In some embodiments, a sample collected at a first time point is a tissue sample or a blood sample, and a sample collected at a subsequent time point (such as a second time point) is a blood sample. In some embodiments, a sample collected at a first time point is a tissue sample and a sample collected at a subsequent time point (such as a second time point) is a blood sample. By monitoring cell types and identifying differences between cell types in samples collected from a subject at two or more timepoints, the present methods can be used, for example, to determine the presence or absence of a condition (such as a cancer), a response of the subject to a treatment, one or more characteristic of a condition (such as a cancer stage) in the subject, recurrence of a condition (such as a cancer), and/or a subject's risk of developing a condition (such as a cancer). Thus, in some embodiments, methods are provided wherein quantities of cell types present in at least one sample (such as at least one whole blood sample, buffy coat sample, leukapheresis sample, or PBMC sample) collected from a subject at one or more timepoints (such as prior to receiving a treatment) are compared to quantities of cell types present in at least one sample collected from the subject at one or more different time points (such as after receiving the treatment). The disclosed methods can allow for patient-specific monitoring, such that, for example, differences in cell type quantities between samples collected from the subject at different timepoints may indicate changes (such as presence or absence of a condition, response to a treatment, a prognosis, or the like) that are significant with respect to the subject but may yet fall within a normal range of a general healthy population.

As disclosed herein, methods are provided for monitoring one or more aspects of a condition in a subject over time, such as but not limited to, a subject's response to receiving a treatment for a condition (such as a response to a chemotherapeutic or immunotherapeutic). In some embodiments, methods are provided for monitoring a response (such as a change in disease state, such as a presence or absence of a metastasis in a subject, such as measured by assessing a presence or level of one or more target regions released from a potential metastasis site in a sample from the subject) of a subject to a treatment (such as a chemotherapy or an immunotherapy). In certain embodiments, one or more samples is collected from the subject at at least 1-10, at least 1-5, at least 2-5, or at least 1, at least 2, least 3, at least 4, at least 5, at least 6, at least 7, at least 8, at least 9, at least 10, at least 15, or at least 20 time points prior to the subject receiving the treatment. In certain embodiments, one or more samples is collected from the subject at at least 1-10, at least 1-5, at least 2-5, or at least 1, at least 2, least 3, at least 4, at least 5, at least 6, at least 7, at least 8, at least 9, at least 10, at least 15, or at least 20 time points after the subject has received the treatment. Sample collection from a subject can be ongoing during and/or after treatment to monitor the subject's response to the treatment.

In some embodiments, samples are not collected from a subject prior to diagnosis of a condition (such as a cancer) or prior to receiving a treatment. In such embodiments, wherein the response of a subject to a treatment, or the course or stage of a condition (such as a cancer) in the subject is being monitored over time, cell types are compared between samples taken at at least 2-10, at least 2-5, at least 3-6, or at least 2, such as at least 3, at least 4, at least 5, at least 6, at least 7, at least 8, at least 9, at least 10, at least 15, or at least 20 time points collected after the subject has been diagnosed and/or after the subject has received the treatment. Sample collection from a subject can be ongoing during and/or after treatment to monitor the subject's response to the treatment.

In some embodiments of the disclosed methods, one or more samples comprising cells (such as one or more tissue, whole blood, buffy coat, leukapheresis, or PBMC samples) is collected from a subject at least once per year, such as about 1-12 times or about 2-6 times, such as about 1, 2, 3, 4, 5, 6, 7, 8, 9, 10, 11, or 12 times per year. In other embodiments, one or more samples is collected from the subject less than once per year, such as about once every 13, 14, 15, 16, 17, 18, 19, 20, 21, 22, 23, or 24 months. In some embodiments, one or more samples is collected from the subject about once every 1-5 years or about once every 1-2 years, such as about every 1, 1.5, 2, 2.5, 3, 3.5, 4, 4.5, or 5 years.

In other embodiments of the disclosed methods, one or more samples comprising cells (such as one or more tissue samples or blood samples, e.g., or one or more buffy coat samples, whole blood samples, leukapheresis samples, or PBMC samples) are collected from a subject at least once per week, such as on 1-4 days, 1-2 days, or on 1, 2, 3, 4, 5, 6, or 7 days per week. In certain embodiments, one or more samples is collected from the subject at least once per month, such as 1-15 times, 1-10 times, 2-5 times, or 1, 2, 3, 4, 5, 6, 7, 8, 9, 10, 11, 12, 13, 14, or 15 times per month. In other embodiments, one or more samples is collected from the subject every month, every 2 months, every 3 months, every 4 months, every 5 months, every 6 months, every 7 months, every 8 months, every 9 months, every 10 months, every 11 months, or every 12 months. In some embodiments, one or more samples is collected from the subject at least once per day, such as 1, 2, 3, 4, 5, or 6 times per day. Selection of the one or more sample collection timepoints (e.g., the frequency of sample collection), or of the number of samples to be collected at each timepoint, depends upon the use to which the methods described herein are to be put by, for example, a research scientist or a clinician (such as a physician).

### 4. Therapies and Related Administration

In certain embodiments, the methods disclosed herein relate to identifying and administering therapies, such as customized therapies, to patients or subjects. In some embodiments, determination of the levels of particular immune cell types, including rare immune cell types, facilitates selection of appropriate treatment. In some embodiments, the patient or subject has a given disease, disorder or condition, e.g., any of the cancers or other conditions described elsewhere herein. Essentially any cancer therapy (e.g., surgical therapy, radiation therapy, chemotherapy, immunotherapy, and/or the like) may be included as part of these methods. In certain embodiments, the therapy administered to a subject comprises at least one chemotherapy drug. In some embodiments, the chemotherapy drug may comprise alkylating agents (for example, but not limited to, Chlorambucil, Cyclophosphamide, Cisplatin and Carboplatin), nitrosoureas (for example, but not limited to, Carmustine and Lomustine), anti-metabolites (for example, but not limited to, Fluorauracil, Methotrexate and Fludarabine), plant alkaloids and natural products (for example, but not limited to, Vincristine, Paclitaxel and Topotecan), anti-tumor antibiotics (for example, but not limited to, Bleomycin, Doxorubicin and Mitoxantrone), hormonal agents (for example, but not limited to, Prednisone, Dexamethasone, Tamoxifen and Leuprolide) and biological response modifiers (for example, but not limited to, Herceptin and Avastin, Erbitux and Rituxan). In some embodiments, the chemotherapy administered to a subject may comprise FOLFOX or FOLFIRI. In certain embodiments, a therapy may be administered to a subject that comprises at least one PARP inhibitor. In some embodiments, the therapies are PARP inhibitors, such as Olaparib (LYNPARZA®), Rucaparib (RUBR ACA®), Niraparib (ZEJULLA®), and Talazoparib (TALZENNA®). These may be used for treating mutations in BRCA1, BRCA2, ATM, BARD1, BRIP1, CDK12, CHEK1, CHEK2, FANCL, PALB2, RAD51B, RAD51 C, RAD51D and RAD541 alterations., and/or for genes associated Homologous Recombination Repair (11RR). Typically, therapies include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

In some embodiments, therapy is customized based on the status of a nucleic acid variant as being of somatic or germline origin. In some embodiments, essentially any cancer therapy (e.g., surgical therapy, radiation therapy, chemotherapy, immunotherapy, and/or the like) may be included as part of these methods. Customized therapies can include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

In some embodiments, the immunotherapy or immunotherapeutic agent targets an immune checkpoint molecule. Certain tumors are able to evade the immune system by co-opting an immune checkpoint pathway. Thus, targeting immune checkpoints has emerged as an effective approach for countering a tumor's ability to evade the immune system and activating anti-tumor immunity against certain cancers. Pardoll, Nature Reviews Cancer, 2012, 12:252-264.

In certain embodiments, the immune checkpoint molecule is an inhibitory molecule that reduces a signal involved in the T cell response to antigen. For example, CTLA4 is expressed on T cells and plays a role in downregulating T cell activation by binding to CD80 (aka B7.1) or CD86 (aka B7.2) on antigen presenting cells. PD-1 is another inhibitory checkpoint molecule that is expressed on T cells. PD-1 limits the activity of T cells in peripheral tissues during an inflammatory response. In addition, the ligand for PD-1 (PD-L1 or PD-L2) is commonly upregulated on the surface of many different tumors, resulting in the downregulation of anti-tumor immune responses in the tumor microenvironment. In certain embodiments, the inhibitory immune checkpoint molecule is CTLA4 or PD-1. In other embodiments, the inhibitory immune checkpoint molecule is a ligand for PD-1, such as PD-L1 or PD-L2. In other embodiments, the inhibitory immune checkpoint molecule is a ligand for CTLA4, such as CD80 or CD86. In other embodiments, the inhibitory immune checkpoint molecule is lymphocyte activation gene 3 (LAG3), killer cell immunoglobulin like receptor (KIR), T cell membrane protein 3 (TIM3), galectin 9 (GAL9), or adenosine A2a receptor (A2aR).

Antagonists that target these immune checkpoint molecules can be used to enhance antigen-specific T cell responses against certain cancers. Accordingly, in certain embodiments, the immunotherapy or immunotherapeutic agent is an antagonist of an inhibitory immune checkpoint molecule. In certain embodiments, the inhibitory immune checkpoint molecule is PD-1. In certain embodiments, the inhibitory immune checkpoint molecule is PD-L1. In certain embodiments, the antagonist of the inhibitory immune checkpoint molecule is an antibody (e.g., a monoclonal antibody). In certain embodiments, the antibody or monoclonal antibody is an anti-CTLA4, anti-PD-1, anti-PD-L1, or anti-PD-L2 antibody. In certain embodiments, the antibody is a monoclonal anti-PD-1 antibody. In some embodiments, the antibody is a monoclonal anti-PD-L1 antibody. In certain embodiments, the monoclonal antibody is a combination of an anti-CTLA4 antibody and an anti-PD-1 antibody, an anti-CTLA4 antibody and an anti-PD-L1 antibody, or an anti-PD-L1 antibody and an anti-PD-1 antibody. In certain embodiments, the anti-PD-1 antibody is one or more of pembrolizumab (Keytruda®) or nivolumab (Opdivo®). In certain embodiments, the anti-CTLA4 antibody is ipilimumab (Yervoy®). In certain embodiments, the anti-PD-L1 antibody is one or more of atezolizumab (Tecentriq®), avelumab (Bavencio®), or durvalumab (Imfinzi®).

In certain embodiments, the immunotherapy or immunotherapeutic agent is an antagonist (e.g. antibody) against CD80, CD86, LAG3, KIR, TIM3, GAL9, or A2aR. In other embodiments, the antagonist is a soluble version of the inhibitory immune checkpoint molecule, such as a soluble fusion protein comprising the extracellular domain of the inhibitory immune checkpoint molecule and an Fc domain of an antibody. In certain embodiments, the soluble fusion protein comprises the extracellular domain of CTLA4, PD-1, PD-L1, or PD-L2. In some embodiments, the soluble fusion protein comprises the extracellular domain of CD80, CD86, LAG3, KIR, TIM3, GAL9, or A2aR. In one embodiment, the soluble fusion protein comprises the extracellular domain of PD-L2 or LAG3.

In certain embodiments, the immune checkpoint molecule is a co-stimulatory molecule that amplifies a signal involved in a T cell response to an antigen. For example, CD28 is a co-stimulatory receptor expressed on T cells. When a T cell binds to antigen through its T cell receptor, CD28 binds to CD80 (aka B7.1) or CD86 (aka B7.2) on antigen-presenting cells to amplify T cell receptor signaling and promote T cell activation. Because CD28 binds to the same ligands (CD80 and CD86) as CTLA4, CTLA4 is able to counteract or regulate the co-stimulatory signaling mediated by CD28. In certain embodiments, the immune checkpoint molecule is a co-stimulatory molecule selected from CD28, inducible T cell co-stimulator (ICOS), CD137, OX40, or CD27. In other embodiments, the immune checkpoint molecule is a ligand of a co-stimulatory molecule, including, for example, CD80, CD86, B7RP1, B7-H3, B7-H4, CD137L, OX40L, or CD70.

Agonists that target these co-stimulatory checkpoint molecules can be used to enhance antigen-specific T cell responses against certain cancers. Accordingly, in certain embodiments, the immunotherapy or immunotherapeutic agent is an agonist of a co-stimulatory checkpoint molecule. In certain embodiments, the agonist of the co-stimulatory checkpoint molecule is an agonist antibody and preferably is a monoclonal antibody. In certain embodiments, the agonist antibody or monoclonal antibody is an anti-CD28 antibody. In other embodiments, the agonist antibody or monoclonal antibody is an anti-ICOS, anti-CD137, anti-OX40, or anti-CD27 antibody. In other embodiments, the agonist antibody or monoclonal antibody is an anti-CD80, anti-CD86, anti-B7RP1, anti-B7-H3, anti-B7-H4, anti-CD137L, anti-OX40L, or anti-CD70 antibody.

Therapeutic agents such as immunotherapeutic agents can function by helping the immune system destroy cancer cells. For example, certain targeted therapeutic agents may mark cancer cells for the immune system to destroy them. Other targeted therapeutic agents may support the immune system to work more effectively against cancer. Yet other therapeutic agents may stop cancer cells from growing, for example, by interfering with cancer cell surface markers preventing them from dividing. Additionally, therapeutic agents can inhibit signals that promote angiogenesis. Such angiogenesis inhibitors prevent blood supply into the tumor thereby, preventing tumor growth. Other targeted therapeutic agents can deliver toxic substances to the tumor. Examples include monoclonal antibodies combined with toxins, chemotherapy, or radiation. Some targeted therapeutic agents induce apoptosis or deplete cancer of hormones. In some embodiments, the therapeutic agents comprise one or more of PARP inhibitors, such as Olaparib (LYNPARZA®), Rucaparib (RUBRACA®), Niraparib (ZEJULA®), and Talazoparib (TALZENNA®). These may be used for treating mutations in BRCA1, BRCA2, ATM, BARD1, BRIP1, CDK12, CHEK1, CHEK2, FANCL, PALB2, RAD51B, RAD51 C, RAD51D and RAD54L alterations, and/or for genes associated Homologous Recombination Repair (HRR).

In some embodiments the treatment comprises one or more immunotherapies, immunotherapeutic agents and/or immune checkpoint inhibitors (ICIS). Immunotherapies are treatments with one or more agents that act to stimulate the immune system so as to kill or at least to inhibit growth of cancer cells, and preferably to reduce further growth of the cancer, reduce the size of the cancer and/or eliminate the cancer. Some such agents bind to a target present on cancer cells; some bind to a target present on immune cells and not on cancer cells; some bind to a target present on both cancer cells and immune cells. Such agents include, but are not limited to, checkpoint inhibitors and/or antibodies. Checkpoint inhibitors are inhibitors of pathways of the immune system that maintain self-tolerance and modulate the duration and amplitude of physiological immune responses in peripheral tissues to minimize collateral tissue damage (see, e.g., Pardoll, Nature Reviews Cancer 12, 252-264 (2012)). Exemplary agents include antibodies against any of PD-1, PD-2, PD-L1, PD-L2, CTLA-4, OX40, B7.1, B7He, LAG3, CD137, KIR, CCR5, CD27, CD40, or CD47. Other exemplary agents include proinflammatory cytokines, such as IL-1, IL-6, and TNF-α. Other exemplary agents are T-cells activated against a tumor, such as T-cells activated by expressing a chimeric antigen targeting a tumor antigen recognized by the T-cell. In some embodiments, anti-PD-1 or anti-PD-L1 therapies comprise pembrolizumab (KEYTRUDA®), nivolumab (OPDIVO®), and cemiplimab (LIBTAYO®), atezolizumab (TECENTRIQ®), durvalumab (INFINZI®), and avelumab (BAVENCIO®). These therapies may be used to treat patients identified as having high microsatellite instability (MSI) status or high tumor mutational burden (TMB).

In some embodiments, a therapeutic agent targets a mutated form of the EGFR protein. Such therapeutic agents can include osimertinib (TAGRISSO®), erlotinib (TARCEVA®), and gefinitib (IRESSA®).

Therapeutic agents can include one or more targeted therapeutic agents, including any one or more of abernaciclib (VERZENIO®), abiraterone acetate (ZYTIGA®), acalabrutinib (CALQUENCE®), adagrasib (KRAZATI®), ado-trastuzumab emtansine (KADCYLA®), afatinib dinaleate (GILOTRIF®), alectinib (ALCENSA®), alemtuzumab (CAMPATH®), alitretinoin (PANRETIN®), alpelisib (PIQRAY®), amivantamab-vmjww (RYBREVANT®), anastrozole (ARIMIDEX®), apalutamide (ERLEADA®), asciminib hydrochloride (SCEMRLIX®), atezolizumab (TECENTRIQ®), avapritinib (AYVAKIT®), avelumab (BAVENCIO®), axicabtagene ciloleucel (YESCARTA®), axitinib (INLYTA®), belinostat (BELEODAQ®), belzutifan (WELIREG®), bevacizumab (AVASTIN®), bexarotene (TARGRETIN®), binimetinib (MEKTOVI®) blinatumomab (BLINCYTO®), bortezomib (VELCADE®), bosutinib (BOSULIF®), brentuximab vedotin (ADCETRIS®), brexucabtagene autoleucel (TIECARTUS®), brigatinib (ALUNBRIG®), cabazitaxel (JEVTANA), cabozantinib-s-malate (CABOMETYX®), cabozantinib-s-malate (COMETRIQ®), capmatinib hydrochloride (TABRECTA®), carfilzomib (KYPROLIS®), cemiplimab-rwlc (LIIBTAYO®), ceritinib (ZYKADIA®), cetuximab (ERBITUX®), ciltacabtagene autoleucel (CARVYKTI®), cobimetinib fumarate (COTELLIC®), copanlisib hydrochloride (ALIQUOPA®), crizotinib (XALKORI®), dabrafenib (TAFMLAR®), dabrafenib mesylate (TAFMLAR®), dacomitinib (VIZIMPRO®), daratumumab (DARZALEX®), daratumumab and hyaluronidase-fihj (DARZALEX FASPRO®), darolutamide (NU-BEQA®), dasatinib (SPRYCEL®), denileukin diftitox (ONTAK®), denosumab (XGEVA®), dinutuximab (UNITUXIN®), dostarlimab-gxly (JEMPERLI®), durvalumab (IMFINZI®), duvelisib (COPIKTRA®), elacestrant dihydrochloride (ORSERDU®), elotuzumab (EMPLICITI®), enasidenib mesylate (IDHIFA®), encorafenib (BRAFTOVI®), enfortumab vedotin-ejfv (PADCEV®), entrectinib (ROZLYTREK®), enzalutamide (XTANDI,), erdafitinib (BALVERSA®), erlotinib hydrochloride (TARCEVA®), everolimus (AFINITOR®), exemestane (AROMASIN®), fain-trastuzumab deruxtecan-nxki (ENHERTU®), fedratinib hydrochloride (INREBIC®), fulvestrant (FASLODEX®), futibatinib (LYTGOBI®), gefitinib (IRESSA®), gemtuzumab ozogamicin (MYLOTARG®), gilteritinib fumarate (XOSPATA®), glasdegib maleate (DAURISMO®), ibritumomab tiuxetan (ZEVALIN®), ibrutinib (IMBRUVICA®), idecabtagene vicleucel (ABECMA®), idelalisib (ZYDELIG®), imatinib mesylate (GLEEVEC®), infigratinib phosphate (TRUSELTIQ®), inotuzumab ozogamicin (BESPONSA®), iobenguane 1 131 (AZEDRA®), ipilimumab (YERVOY®), isatuximab-irfe (SARCLISA®), ivosidenib (TIBSOVO®), ixazomib citrate (NINLARO®), lanreotide acetate (SOMATULINE DEPOT®), lapatinib ditosylate (TYKERB®), larotrectinib sulfate (VITRAKVI®), lenvatinib mesylate (LENVIMA®) letrozole (FEMARA®), lisocabtagene maraleucel (BREYANZI®), loncastuximab tesirine-lpyl (ZYNLONTA®), lorlatinib (LORBRENA®), lutetium Lu 177 vipivotide tetraxetan (PLUVICTO®), lutetium Lu 177-dotatate (LUTATHRA®), niargetuximab-cmkb (MARGENZA®), midostaurin (RYDAPT®), mirvetuximab soravtansine-gynx (ELAHERE®), mobocertinib succinate (EXKIVITY®), mogamulizumab-kpkc (POTELIGEO®), mosunetuzumab-axgb (LUNSUMIO®), moxetumomab pasudotox-tdfk (LUMOXITI®), naxitamab-gqgk (DANYELZA®), necitumumab (PORTRAZZA®), neratinib maleate (NERLYNX®), nilotinib (TASIGNA®), niraparib tosylate monohydrate (ZEJULA®), nivolumab (OPDIVO®), nivolumab and relatlimab-rmbw (OPDUALAG®), obinutuzumab (GAZYVA®), ofatumumab (ARZERRA®), olaparib (LYNPARZA®), olutasidenib (REZLHIDIA®), osimertinib mesylate (TAGRISSO®), pacritinib citrate (VONJO®), palbociclib (IBRANCE®), panitumumab (VECTIBIX®), pazopanib hydrochloride (VOTRIENT®), pembrolizumab (KEYTRUDA®), pemigatinib (PEMAZYRE®), pertuzumab (PERJETA®), pertuzumab, trastuzumab, and hyaluronidase-zzxf (PHESGO®), pexidartinib hydrochloride (TURALIO®), pirtobrutinib (JAYPIRCA®), polatuzumab vedotin-piiq (POLIVY®), ponatinib hydrochloride (ICLUSIG®), pralatrexate (FOLOTYN®), pralsetinib (GAVRETO®), radium 223 dichloride (XOFIGO®), ramucirumab (CYRAMZA®), regorafenib (STIVARGA®), retifanlimab-dlwr (ZYNYZ®), ribociclib (KISQALI®), ripretinib (QINLOCK®), rituximab (RITUXAN®), rituximab and hyaluronidase human (RITUXAN HYCELA®), romidepsin (ISTODAX®), rucaparib camsylate (RUBRACA®), ruxolitinib phosphate (JAKAFI®), sacituzumab govitecan-hziy (TRODELVY®), selinexor (XPOVIO®), selpercatinib (RETEVMO®), selumetinib sulfate (KOSELUGO®), siltuximab (SYLVANT®), sirolimus protein-bound particles (FYARRO®), sonidegib (ODOMZO®), sorafenib tosylate (NEXAVAR®), sotorasib (LUMAKRAS®), sunitinib malate (SUTENT®), tafasitamab-cxix (MONJUVI®), tagraxofusp-erzs (ELZONRIS®), talazoparib tosylate (TALZENNA®), tamoxifen citrate (SOLTAMOX®), tazemetostat hydrobromide (TAZVERIK®), tebentafusp-tebn (KIMMTRAK®), teclistamab-cqyv (TECVAYLI®), temsirolimus (TORISEL®), tepotinib hydrochloride (TEPMETKO®), tisagenlecleucel (KYMRIAH®), tisotumab vedotin-tftv (TIVDAK®), tivozanib hydrochloride (FOTIVDA®), toremifene (FARESTON®), trametinib (MEKINIST®), trametinib dimethyl sulfoxide (MEKINIST®), trastuzumab (HERCEPTIN®), tremelimumab-actl (IMJUDO®), tretinoin (VESANOID®), tucatinib (TUKYSA®), vandetanib (CAPRELSA®), vemurafenib (ZELBORAF®), venetoclax (VENCLEXTA®), vismodegib (ERIVEDGE®), vorinostat (ZOLINZA®), zanubrutinib (BRUKINSA®), and/or ziv-aflibercept (ZALTRAP®).

Table 6 provides an exemplary list of drugs used to treat cancers with mutations observed in target genes associated with certain cancer types. In certain embodiments, the subject has a cancer of a type listed in Table 6 including a mutation in one or more target genes listed in Table 6 for that cancer type, and the therapy administered to the subject comprises the drug listed in Table 6 for that cancer type and mutation.

TABLE 6
Exemplary drugs

| Cancer type | Drug | Target genes |
|---|---|---|
| Breast | abemaciclib (VERZENIO ®) | CDK4, CDK6 |
| prostate | abiraterone acetate (ZYTIGA ®) | CYP17A1 |
| leukemia | acalabrutinib (CALQUENCE ®) | BTK |
| lymphoma | acalabrutinib (CALQUENCE ®) | BTK |
| lung | adagrasib (KRAZATI ®) | KRAS G12C |
| Breast | ado-trastuzumab emtansine | Tubulin, Her2 |
| | (KADCYLA ®) | |
| lung | afatinib dimaleate (GILOTRIFR) | EGFR + EGFR exon 21 L858R |
| | | mutation + EGFR-Ex19del + HER2 + |
| | | HER4 |
| lung | alectinib (ALECENSAR) | ALK, RET |
| leukemia | alemtuzumab (CAMPATH ®) | CD52 |
| skin | alitretinoin (PANRENTIN ®) | RARα + RARβ2 + RARγ |
| soft tissue | alitretinoin (PANRENTIN ®) | RARα + RARβ2 + RARγ |
| sarcoma | | |
| Breast | alpelisib (PIQRAY ®) | PI3Kα |
| lung | amivantamab-vmjw (RYVEBRANT ®) | EGFR+c-MET |
| Breast | anastrozole (ARIMIDEX ®) | Aromatase |
| prostate | apalutamide (ERLEADA ®) | AR |
| leukemia | asciminib hydrochloride (SCEMBLIX ®) | Bcr-Abl |
| bladder | atezolizumab (TECENTRIQ ®) | PDL1 |
| liver and bile | atezolizumab (TECENTRIQ ®) | PDL1 |
| duct | | |
| lung | atezolizumab (TECENTRIQ ®) | PDL1 |
| skin | atezolizumab (TECENTRIQ ®) | PDL1 |
| soft tissue | atezolizumab (TECENTRIQ ®) | PDL1 |
| sarcoma | | |
| Gastrointestinal | avapritinib (AYVAKIT ®) | PDGFRα + c-Kit |
| systemic | avapritinib (AYVAKIT ®) | PDGFRα + c-Kit |
| mastocytosis | | |
| bladder | avelumab (BAVENCIO ®) | PDL1 |
| endocrine and | avelumab (BAVENCIO ®) | PDL1 |
| neuroendocrine | | |
| tumors | | |
| kidney | avelumab (BAVENCIO ®) | PDL1 |
| skin | avelumab (BAVENCIO ®) | PDL1 |
| lymphoma | axicabtagene ciloleucel (YESCARTA ®) | CD19 |
| kidney | axitinib (INLYTA ®) | VEGFR1 + VEGFR2 + VEGFR3 |
| lymphoma | belinostat (BELEODAQ ®) | HDAC |
| Brain | belzutifan (WELIREGR) | HIF-2A |
| kidney | belzutifan (WELIREG ®) | HIF-2A |
| pancreatic | belzutifan (WELIREG ®) | HIF-2A |
| ovarian | bevacizumab (AVASTIN ®) | VEGF-A |
| epithelial, | | |
| fallopian tube, | | |
| and primary | | |
| peritoneal | | |
| Brain | bevacizumab (AVASTIN ®) | VEGF-A |
| Cervical | bevacizumab (AVASTIN ®) | VEGF-A |
| colorectal | bevacizumab (AVASTIN ®) | VEGF-A |
| kidney | bevacizumab (AVASTIN ®) | VEGF-A |
| liver and bile | bevacizumab (AVASTIN ®) | VEGF-A |
| duct | | |
| lung | bevacizumab (AVASTIN ®) | VEGF-A |
| lymphoma | bexarotene (TARGRETIN ®) | RXRs |
| skin | binimetinib (MEKTOVI ®) | MEK1, MEK2 |
| leukemia | blinatumomab (BLINCYTO ®) | CD19, CD3 |
| lymphoma | bortezomib (VELCADE ®) | Proteasome |
| multiple | bortezomib (VELCADE ®) | Proteasome |
| myeloma | | |
| leukemia | bosutinib (BOSULIF ®) | BCR-ABL, SRC |
| lymphoma | brentuximab vedotin (ADCETRIS ®) | CD130, Tubulin |
| leukemia | brexucabtagene autoleucel (TECARTUS ®) | CD19 |
| lymphoma | brexucabtagene autoleucel (TECARTUS ®) | CD19 |
| lung | brigatinib (ALUNBRIG ®) | ALK, EGFR, FLT3, IGF-1R, ROS1 |
| prostate | cabazitaxel (JEVTANA ®) | Tubulin |
| kidney | cabozantinib-s-malate (CABOMETYX ®) | AXL + RET + ROS1 + TYRO3 + |
| | | Tie-2 + TrkB + VEGFR1 + VEGFR2 + |
| | | VEGFR3 + c-Kit + c-Met |
| liver and bile | cabozantinib-s-malate (CABOMETYX ®) | AXL + RET + ROS1 + TYRO3 + |
| duct | | Tie-2 + TrkB + VEGFR1 + VEGFR2 + |
| | | VEGFR3 + c-Kit + c-Met |
| thyroid | cabozantinib-s-malate (COMETRIQ ®) | AXL+ RET+ ROS1 + TYRO3 + |
| | | Tie-2 + TrkB + VEGFR1 + VEGFR2 + |
| | | VEGFR3 + c-Kit + c-Met |
| lung | capmatinib hydrochloride (TABRECTA ®) | c-Met |
| multiple | carfilzomib (KYPROLIS ®) | protoasome |
| myeloma | | |
| lung | cemiplimab-rwlc (LIBTAYO ®) | PD-1 |
| skin | cemiplimab-rwlc (LIBTAYO ®) | PD-1 |
| lung | ceritinib (ZYKADIA ®) | ALK + IGF-1R + INSR + ROS1 |
| colorectal | cetuximab (ERBITUX ®) | EGFR |
| multiple | ciltacabtagene autoleucel (CARVYKTI ®) | BCMA |
| myeloma | | |
| skin | cobimetinib fumarate (COTELLIC ®) | MEK1, MEK2 |
| lymphoma | copanlisib hydrochloride (ALIQOPA ®) | PI3Kα, PI3Kδ |
| lung | crizotinib (XALKORI ®) | MTH1 |
| lymphoma | crizotinib (XALKORI ®) | MTH1 |
| Myofibroblastic | crizotinib (XALKORI ®) | MTH1 |
| Brain | dabrafenib (TAFINLAR ®) | BRAF, CRAF |
| lung | dabrafenib mesylate (TAFINLAR ®) | BRAF, CRAF |
| skin | dabrafenib mesylate (TAFINLAR ®) | BRAF, CRAF |
| solid tumors | dabrafenib mesylate (TAFINLAR ®) | BRAF, CRAF |
| anywhere in the | | |
| body | | |
| thyroid | dabrafenib mesylate (TAFINLAR ®) | BRAF, CRAF |
| lung | dacomitinib (VIZIMPRO ®) | EGFR + EGFR exon 21 L858R |
| | | mutation + EGFR-Ex19del + HER2 + |
| | | HER4 |
| multiple | daratumumab (DARZALEX ®) | CD38 + Hyaluronic acid |
| myeloma | | |
| multiple | daratumumab and hyaluronidase-fihj | CD38 + Hyaluronic acid |
| myeloma | (DARZALEX FASPRO ®) | |
| prostate | darolutamide (NUBEQA ®) | AR |
| leukemia | dasatinib (SPRYCEL ®) | Bcr-Abl + EphA2 + FYN + LCK + |
| | | PDGFRβ + Protein-tyrosine kinases + |
| | | SRC + YES1 + c-Kit |
| neuroblastoma | dinutuximab (UNITUXIN ®) | GD2 |
| endometrial | dostarlimab-gxly (JEMPERLI ®) | PD-1 |
| solid tumors | dostarlimab-gxly (JEMPERLI ®) | PD-1 |
| anywhere in the | | |
| body | | |
| liver and bile | durvalumab (IMFINZI ®) | PDL1 |
| duct | | |
| lung | durvalumab (IMFINZI ®) | PDL1 |
| leukemia | duvelisib (COPIKTRA ®) | PI3Kγ + PI3Kδ |
| Breast | elacestrant dihydrochloride (ORSERDU ®) | Erα/ESR1 |
| multiple | elotuzumab (EMPLICITI ®) | SLAMF7 |
| myeloma | | |
| leukemia | enasidenib mesylate (IDHIFA ®) | IDH2 |
| colorectal | encorafenib (BRAFTOVI ®) | BRAF, BRAF V600E |
| skin | encorafenib (BRAFTOVI ®) | BRAF V600E, V600K mutation |
| bladder | enfortumab vedotin-ejfv (PADCEV ®) | Nectin-4, Tubulins |
| lung | entrectinib (ROZLYTREK ®) | ALK + ROS1 + TRKA + TrkB + |
| | | TrkC |
| solid tumors | entrectinib (ROZLYTREK ®) | ALK + ROS1 + TRKA + TrkB + |
| anywhere in the | | TrkC |
| body | | |
| prostate | enzalutamide (XTANDI ®) | AR |
| bladder | erdafitinib (BALVERSA ®) | FGFR1, FGFR2, FGFR3, FGFR4 |
| lung | erlotinib hydrochloride (TARCEVA ®) | EGFR antagonists, EGFR exon 21 |
| | | L858R mutation inhibitors, EGFR- |
| | | Ex19del inhibitors |
| pancreatic | erlotinib hydrochloride (TARCEVA ®) | EGFR + EGFR exon 21 L858R |
| | | mutation + EGFR-Ex19del |
| Breast | everolimus (AFINITOR ®) | mTORC1, mTORC2 |
| kidney | everolimus (AFINITOR ®) | mTORC1, mTORC2 |
| pancreatic | everolimus (AFINITOR ®) | mTORC1, mTORC2 |
| Breast | exemestane (AROMASIN ®) | Aromatase |
| Breast | fam-trastuzumab deruxtecan-nxki | HER2, TOP1 |
| | (ENHERTU ®) | |
| Gastric | fam-trastuzumab deruxtecan-nxki | HER2, TOP1 |
| | (ENHERTU ®) | |
| lung | fam-trastuzumab deruxtecan-nxki | HER2, TOP1 |
| | (ENHERTU ®) | |
| myelodysplasti | fedratinib hydrochloride (INREBIC ®) | JAK2, FLT3 |
| c and | | |
| myeloproliferat | | |
| ive disorders | | |
| Breast | fulvestrant (FASLODEX ®) | ER |
| liver and bile | futibatinib (LYTGOBI ®) | FGFRs |
| duct | | |
| lung | gefitinib (IRESSA ®) | EGFR + EGFR exon 21 L858R |
| | | mutation + EGFR-Ex19del |
| leukemia | gemtuzumab ozogamicin (MYLOTARG ®) | CD33, DNA |
| leukemia | gilteritinib fumarate (XOSPATA ®) | AXL + FLT3 |
| leukemia | glasdegib maleate (DAURISMO ®) | SMO |
| lymphoma | ibritumomab tiuxetan (ZEVALIN ®) | CD20 |
| leukemia | ibrutinib (IMBRUVICA ®) | BTK |
| lymphoma | ibrutinib (IMBRUVICA ®) | BTK |
| multiple | idecabtagene vicleucel (ABECMA ®) | BCMA |
| myeloma | | |
| leukemia | idelalisib (ZYDELIG ®) | PI3Kδ |
| dermatofibrosar | imatinib mesylate (GLEEVEC ®) | BCR-ABL, PDGFR, C-Kit |
| coma | | |
| protuberans | | |
| Gastrointestinal | imatinib mesylate (GLEEVEC ®) | BCR-ABL, PDGFR, C-Kit |
| leukemia | imatinib mesylate (GLEEVEC ®) | BCR-ABL, PDGFR, C-Kit |
| myelodysplasti | imatinib mesylate (GLEEVEC ®) | BCR-ABL, PDGFR, C-Kit |
| c and | | |
| myeloproliferat | | |
| ive disorders | | |
| systemic | imatinib mesylate (GLEEVEC ®) | BCR-ABL, PDGFR, C-Kit |
| mastocytosis | | |
| liver and bile | infigratinib phosphate (TRUSELTIQ ®) | FGFR1 + FGFR2 + FGFR3 + FGFR4 |
| duct | | |
| leukemia | inotuzumab ozogamicin (BESPONSA ®) | CD22 + DNA |
| endocrine and | iobenguane I 131 (AZEDRA ®) | NET |
| neuroendocrine | | |
| tumors | | |
| colorectal | ipilimumab (YERVOY ®) | CTLA4 |
| esophageal | ipilimumab (YERVOY ®) | CTLA4 |
| kidney | ipilimumab (YERVOY ®) | CTLA4 |
| liver and bile | ipilimumab (YERVOY ®) | CTLA4 |
| duct | | |
| lung | ipilimumab (YERVOY ®) | CTLA4 |
| malignant | ipilimumab (YERVOY ®) | CTLA4 |
| mesothelioma | | |
| skin | ipilimumab (YERVOY ®) | CTLA4 |
| multiple | isatuximab-irfc (SARCLISA ®) | CD38 |
| myeloma | | |
| leukemia | ivosidenib (TIBSOVO ®) | IDH1 |
| multiple | ixazomib citrate (NINLARO ®) | proteasome |
| myeloma | | |
| endocrine and | lanreotide acetate (SOMATULINE | SSTR |
| neuroendocrine | DEPOT ®) | |
| tumors | | |
| Breast | lapatinib ditosylate (TYKERB ®) | EGFR |
| solid tumors | larotrectinib sulfate (VITRAKVI ®) | TRKA + TrkB + TrkC |
| anywhere in the | | |
| body | | |
| endometrial | lenvatinib mesylate (LENVIMA ®) | FGFR1 + FGFR2 + FGFR3 + FGFR4 + |
| | | PDGFRα + RET + VEGFR1 + |
| | | VEGFR2 + VEGFR3 + c-Kit |
| kidney | lenvatinib mesylate (LENVIMA ®) | FGFR1 + FGFR2 + FGFR3 + FGFR4 + |
| | | PDGFRα + RET + VEGFR1 + |
| | | VEGFR2 + VEGFR3 + c-Kit |
| liver and bile | lenvatinib mesylate (LENVIMA ®) | FGFR1 + FGFR2 + FGFR3 + FGFR4 + |
| duct | | PDGFRα + RET + VEGFR1 + |
| | | VEGFR2 + VEGFR3 + c-Kit |
| thyroid | lenvatinib mesylate (LENVIMA ®) | FGFR1 + FGFR2 + FGFR3 + FGFR4 + |
| | | PDGFRα + RET + VEGFR1 + |
| | | VEGFR2 + VEGFR3 + c-Kit |
| Breast | letrozole (FEMARA ®)/Ribociclib | CDK4/ER |
| | Succinate | |
| lymphoma | lisocabtagene maraleucel (BREYANZI ®) | CD19 |
| lymphoma | loncastuximab tesirine-lpyl | CD19 + DNA |
| | (ZYNLONTA ®) | |
| lung | lorlatinib (LORBRENA ®) | ALK + ROS1 |
| prostate | lutetium Lu 177 vipivotide tetraxetan | PSMA |
| | (PLUVICTOR) | |
| endocrine and | lutetium Lu 177-dotatate (LUTATHERA ®) | SSTR2 |
| neuroendocrine | | |
| tumors | | |
| Breast | margetuximab-cmkb (MARGENZAR) | HER2 |
| leukemia | midostaurin (RYDAPT ®) | FLT3 + PDGFR + PKC + Syk + |
| | | VEGFR2 + c-Kit |
| systemic | midostaurin (RYDAPT ®) | FLT3 + PDGFR + PKC + Syk + |
| mastocytosis | | VEGFR2 + c-Kit |
| ovarian | mirvetuximab soravtansine-gynx | FOLR1 + Tubulin |
| epithelial, | (ELAHERE ®) | |
| fallopian tube, | | |
| and primary | | |
| peritoneal | | |
| lung | mobocertinib succinate (EXKIVITY ®) | EGFR exon 20 + HER2 exon 20 |
| lymphoma | mogamulizumab-kpkc (POTELIGEOR) | CCR4 |
| lymphoma | mosunetuzumab-axgb (LUNSUMIO ®) | CD20, CD3 |
| leukemia | moxetumomab pasudotox- | CD22 |
| | tdfk (LUMOXITI ®) | |
| neuroblastoma | naxitamab-gqgk (DANYELZAR) | GD2 |
| lung | necitumumab (PORTRAZZA ®) | EGFR |
| Breast | neratinib maleate (NERLYNX ®) | EGFR, HER2, HER4 |
| leukemia | nilotinib (TASIGNA ®) | Bcr-Abl + CSF-1R + DDR1 + |
| | | PDGFR + c-Kit |
| ovarian | niraparib tosylate monohydrate | PARP1 + PARP2 |
| epithelial, | (ZEJULA ®) | |
| fallopian tube, | | |
| and primary | | |
| peritoneal | | |
| bladder | nivolumab (OPDIVO ®) | PD-1 |
| colorectal | nivolumab (OPDIVO ®) | PD-1 |
| esophageal | nivolumab (OPDIVO ®) | PD-1 |
| kidney | nivolumab (OPDIVO ®) | PD-1 |
| liver and bile | nivolumab (OPDIVO ®) | PD-1 |
| duct | | |
| lung | nivolumab (OPDIVO ®) | PD-1 |
| lymphoma | nivolumab (OPDIVO ®) | PD-1 |
| malignant | nivolumab (OPDIVO ®) | PD-1 |
| mesothelioma | | |
| skin | nivolumab (OPDIVO ®) | PD-1 |
| stomach | nivolumab (OPDIVO ®) | PD-1 |
| (gastric) | | |
| skin | nivolumab and relatlimab-rmbw | LAG3 + PD-1 |
| | (OPDUALAG ®) | |
| leukemia | obinutuzumab (GAZYVA ®) | CD20 |
| lymphoma | obinutuzumab (GAZYVA ®) | CD20 |
| leukemia | ofatumumab (ARZERRA ®) | CD20 |
| ovarian | olaparib (LYNPARZA ®) | PARP1, PARP2, PARP3 |
| epithelial, | | |
| fallopian tube, | | |
| and primary | | |
| peritoneal | | |
| Breast | olaparib (LYNPARZA ®) | PARP1, PARP2, PARP3 |
| pancreatic | olaparib (LYNPARZA ®) | PARP1, PARP2, PARP3 |
| prostate | olaparib (LYNPARZAR) | PARP1, PARP2, PARP3 |
| leukemia | olutasidenib (REZLIDHIA ®) | IDH1 |
| lung | osimertinib mesylate (TAGRISSO ®) | EGFR + EGFR T790M + EGFR exon |
| | | 21 L858R mutation + EGFR-Ex19del |
| myelodysplasti | pacritinib citrate (VONJO ®) | CSF-1R + FLT3 + IRAK1 + JAK2 |
| c and | | |
| myeloproliferat | | |
| ive disorders | | |
| Breast | palbociclib (IBRANCER) | CDK4, CDK6 |
| colorectal | panitumumab (VECTIBIX ®) | EGFR |
| kidney | pazopanib hydrochloride (VOTRIENT ®) | FGFR1 + FGFR3 + Flt3L + ITK + |
| | | LCK + PDGFRα + PDGFRβ + |
| | | VEGFR1 + VEGFR2 + VEGFR3 + c- |
| | | Kit |
| soft tissue | pazopanib hydrochloride (VOTRIENT ®) | FGFR1 + FGFR3 + Flt3L + ITK + |
| sarcoma | | LCK + PDGFRα + PDGFRβ + |
| | | VEGFR1 + VEGFR2 + VEGFR3 + c- |
| | | Kit |
| Breast | pembrolizumab (KEYTRUDA ®) | PD-1 |
| Cervical | pembrolizumab (KEYTRUDA ®) | PD-1 |
| colorectal | pembrolizumab (KEYTRUDA ®) | PD-1 |
| endometrial | pembrolizumab (KEYTRUDA ®) | PD-1 |
| esophageal | pembrolizumab (KEYTRUDAR) | PD-1 |
| kidney | pembrolizumab (KEYTRUDA ®) | PD-1 |
| liver and bile | pembrolizumab (KEYTRUDA ®) | PD-1 |
| duct | | |
| lung | pembrolizumab (KEYTRUDA ®) | PD-1 |
| lymphoma | pembrolizumab (KEYTRUDA ®) | PD-1 |
| skin | pembrolizumab (KEYTRUDA ®) | PD-1 |
| solid tumors | pembrolizumab (KEYTRUDA ®) | PD-1 |
| anywhere in the | | |
| body | | |
| stomach | pembrolizumab (KEYTRUDA ®) | PD-1 |
| (gastric) | | |
| leukemia | pemigatinib (PEMAZYRE ®) | FGFR1, FGFR2, FGFR3, FGFR4 |
| liver and bile | pemigatinib (PEMAZYRER) | FGFR1, FGFR2, FGFR3, FGFR4 |
| duct | | |
| lymphoma | pemigatinib (PEMAZYRER) | FGFR1, FGFR2, FGFR3, FGFR4 |
| myelodysplasti | pemigatinib (PEMAZYRER) | FGFR1, FGFR2, FGFR3, FGFR4 |
| c and | | |
| myeloproliferat | | |
| ive disorders | | |
| Breast | pertuzumab (PERJETA ®) | HER2 |
| Breast | pertuzumab, trastuzumab, and | HER2, Hyaluronic acid |
| | hyaluronidase-zzxf (PHESGO ®) | |
| giant cell tumor | pexidartinib hydrochloride (TURALIO ®) | CSF-1R + FLT3 + c-Kit |
| lymphoma | pirtobrutinib (JAYPIRCAR) | BTK C481S |
| lymphoma | polatuzumab vedotin-piiq (POLIVY ®) | CD79B + Tubulin |
| leukemia | ponatinib hydrochloride (ICLUSIG ®) | Bcr-Abl + Protein-tyrosine kinases |
| lymphoma | pralatrexate (FOLOTYN ®) | Bcr-Abl + Protein-tyrosine kinases |
| lung | pralsetinib (GAVRETO ®) | RET |
| thyroid | pralsetinib (GAVRETO ®) | RET |
| prostate | radium 223 dichloride (XOFIGO ®) | DNA |
| colorectal | ramucirumab (CYRAMZAR) | VEGFR2 |
| Gastric | ramucirumab (CYRAMZA ®) | VEGFR2 |
| liver and bile | ramucirumab (CYRAMZA ®) | VEGFR2 |
| duct | | |
| lung | ramucirumab (CYRAMZA ®) | VEGFR2 |
| stomach | ramucirumab (CYRAMZA ®) | VEGFR2 |
| (gastric) | | |
| colorectal | regorafenib (STIVARGA ®) | Abl family, BRAF, BRAF V600E, |
| | | CRAF, CSF-1R, DDR2, EphA2, |
| | | FGFR1, FGFR2, FRK, |
| | | MAPK11, PDGFRa, PDGFRb, |
| | | RET, TRKA, Tie-2, VEGFR1, |
| | | VEGFR2, VEGFR3, c-kit |
| Gastrointestinal | regorafenib (STIVARGA ®) | Abl family, BRAF, BRAF V600E, |
| | | CRAF, CSF-1R, DDR2, EphA2, |
| | | FGFR1, FGFR2, FRK, |
| | | MAPK11, PDGFRa, PDGFRb, |
| | | RET, TRKA, Tie-2, VEGFR1, |
| | | VEGFR2, VEGFR3, c-kit |
| liver and bile | regorafenib (STIVARGA ®) | Abl family, BRAF, BRAF V600E, |
| duct | | CRAF, CSF-1R, DDR2, EphA2, |
| | | FGFR1, FGFR2, FRK, |
| | | MAPK11, PDGFRa, PDGFRb, |
| | | RET, TRKA, Tie-2, VEGFR1, |
| | | VEGFR2, VEGFR3, c-kit |
| skin | retifanlimab-dlwr (ZYNYZ ®) | PD-1 |
| Breast | ribociclib (KISQALI ®) | CDK4, CDK6 |
| gastrointestinal | ripretinib (QINLOCK ®) | EGFR + PDGFRα + c-Kit |
| leukemia | rituximab (RITUXAN ®) | CD20 + Hyaluronic acid |
| lymphoma | rituximab (RITUXAN ®) | CD20 + Hyaluronic acid |
| leukemia | rituximab and hyaluronidase human | CD20 + Hyaluronic acid |
| | (RITUXAN HYCELA ®) | |
| lymphoma | rituximab and hyaluronidase human | CD20 + Hyaluronic acid |
| | (RITUXAN HYCELA ®) | |
| lymphoma | romidepsin (ISTODAX ®) | HDAC |
| ovarian | rucaparib camsylate (RUBRACA ®) | PARP1 + PARP2 + PARP3 |
| epithelial, | | |
| fallopian tube, | | |
| and primary | | |
| peritoneal | | |
| prostate | rucaparib camsylate (RUBRACA ®) | PARP1 + PARP2 + PARP3 |
| myelodysplasti | ruxolitinib phosphate (JAKAFI ®) | JAK1 + JAK2 |
| c and | | |
| myeloproliferat | | |
| ive disorders | | |
| Breast | sacituzumab govitecan-hziy | TOP1 |
| | (TRODELVY ®) | |
| lymphoma | selinexor (XPOVIOR) | XPO1 |
| multiple | selinexor (XPOVIO ®) | XPO1 |
| myeloma | | |
| lung | selpercatinib (RETEVMO ®) | RET |
| solid tumors | selpercatinib (RETEVMOR) | RET |
| anywhere in the | | |
| body | | |
| thyroid | selpercatinib (RETEVMO ®) | RET |
| plexiform | selumetinib sulfate (KOSELUGO ®) | MEK1 + MEK2 |
| neurofibroma | | |
| soft tissue | sirolimus protein-bound particles | MUT + mTOR |
| sarcoma | (FYARRO ®) | |
| skin | sonidegib (ODOMZO ®) | SMO |
| kidney | sorafenib tosylate (NEXAVAR ®) | BRAF inhibitors, CRAF inhibitors, |
| | | FLT3 + PDGFRβ + RET + VEGFR1 + |
| | | VEGFR2 + VEGFR3 + c-Kit |
| liver and bile | sorafenib tosylate (NEXAVAR ®) | BRAF + CRAF + FLT3 + PDGFRβ + |
| duct | | RET + VEGFR1 + VEGFR2 + |
| | | VEGFR3 + c-Kit |
| thyroid | sorafenib tosylate (NEXAVAR ®) | BRAF + CRAF + FLT3 + PDGFRβ + |
| | | RET + VEGFR1 + VEGFR2 + |
| | | VEGFR3 + c-Kit |
| lung | sotorasib (LUMAKRAS ®) | KRAS G12C |
| Gastrointestinal | sunitinib malate (SUTENT ®) | PDGFR + RTK + VEGFR |
| kidney | sunitinib malate (SUTENT ®) | PDGFR + RTK + VEGFR |
| pancreatic | sunitinib malate (SUTENT ®) | PDGFR + RTK + VEGFR |
| lymphoma | tafasitamab-cxix (MONJUVI ®) | CD19 |
| leukemia | tagraxofusp-erzs (ELZONRIS ®) | CD123 |
| Breast | talazoparib tosylate (TALZENNA ®) | PARP1, PARP2 |
| Breast | tamoxifen citrate (SOLTAMOX ®) | ER |
| lymphoma | tazemetostat hydrobromide | EZH2 |
| | (TAZVERIK ®) | |
| soft tissue | tazemetostat hydrobromide | EZH2 |
| sarcoma | (TAZVERIK ®) | |
| skin | tebentafusp-tebn (KIMMTRAK ®) | CD3 + gp100 |
| multiple | teclistamab-cqyv (TECVAYLI ®) | CD3 + gp100 |
| myeloma | | |
| kidney | temsirolimus (TORISEL ®) | CD3 + gp100 |
| lung | tepotinib hydrochloride (TEPMETKO ®) | MET |
| leukemia | tisagenlecleucel (KYMRIAH ®) | CD19 |
| lymphoma | tisagenlecleucel (KYMRIAH ®) | CD19 |
| Cervical | tisotumab vedotin-tftv (TIVDAK ®) | Tubulin, tissue factor |
| kidney | tivozanib hydrochloride (FOTIVDA ®) | (VEGFR)-1, VEGFR-2 and VEGFR- |
| | | 3, c-kit, and PDGFR β |
| Breast | toremifene (FARESTON ®) | ER |
| Brain | trametinib (MEKINIST ®) | MEK1 and MEK2 |
| lung | trametinib dimethyl sulfoxide | MEK1 and MEK2 |
| | (MEKINIST ®) | |
| skin | trametinib dimethyl sulfoxide | MEK1 and MEK2 |
| | (MEKINIST ®) | |
| solid tumors | trametinib dimethyl sulfoxide | MEK1 and MEK2 |
| anywhere in the | (MEKINIST ®) | |
| body | | |
| thyroid | trametinib dimethyl sulfoxide | MEK1 and MEK2 |
| | (MEKINIST ®) | |
| Breast | trastuzumab (HERCEPTIN ®) | HER2 |
| esophageal | trastuzumab (HERCEPTIN ®) | HER2 |
| stomach | trastuzumab (HERCEPTIN ®) | HER2 |
| (gastric) | | |
| liver and bile | tremelimumab-actl (IMJUDO ®) | CTLA-4, CD80, and CD86 |
| duct | | |
| lung | tremelimumab-actl (IMJUDO ®) | CTLA-4, CD80, and CD86 |
| Breast | tucatinib (TUKYSA ®) | HER2 |
| colorectal | tucatinib (TUKYSA ®) | HER2 |
| thyroid | vandetanib (CAPRELSA ®) | VEGFR-2, EGFR, RET |
| skin | vemurafenib (ZELBORAF ®) | BRAF, BRAF V600E, CRAF, ARAF, |
| | | SRMS, ACK1, MAP4K5, FGR |
| leukemia | venetoclax (VENCLEXTA ®) | BCL-2 |
| lymphoma | venetoclax (VENCLEXTA ®) | BCL-2 |
| lymphoma | vorinostat (ZOLINZA ®) | HDAC1, HDAC2 and HDAC3 (Class |
| | | I) and HDAC6 (Class II) |
| leukemia | zanubrutinib (BRUKINSA R) | BTK |
| lymphoma | zanubrutinib (BRUKINSA ®) | BTK |
| colorectal | ziv-aflibercept (ZALTRAP ®) | PGF, VEGF-A |

In some embodiments, the methods described herein can be used to treat patients by (i) detecting one or more mutations in the one or more target genes listed in Table 6; and (ii) administering the corresponding one or more drugs listed in Table 6. In some embodiments, these therapies may be used alone or in combination with other therapies to treat a disease.

In some embodiments, the methods and systems disclosed herein may be used to identify customized or targeted therapies to treat a given disease or condition in patients based on the classification of a nucleic acid variant as being of somatic or germline origin. In certain embodiments, the status of a nucleic acid variant from a sample from a subject as being of somatic or germline origin may be compared with a database of comparator results from a reference population to identify customized or targeted therapies for that subject. Typically, the reference population includes patients with the same cancer or disease type as the subject and/or patients who are receiving, or who have received, the same therapy as the subject. A customized or targeted therapy (or therapies) may be identified when the nucleic variant and the comparator results satisfy certain classification criteria (e.g., are a substantial or an approximate match).

In certain embodiments, the customized therapies described herein are typically administered parenterally (e.g., intravenously or subcutaneously). Pharmaceutical compositions containing an immunotherapeutic agent are typically administered intravenously. Certain therapeutic agents are administered orally. However, customized therapies (e.g., immunotherapeutic agents, etc.) may also be administered by any method known in the art, for example, buccal, sublingual, rectal, vaginal, intraurethral, topical, intraocular, intranasal, and/or intraauricular, which administration may include tablets, capsules, granules, aqueous suspensions, gels, sprays, suppositories, salves, ointments, or the like.

Therapeutic options for treating specific genetic-based diseases, disorders, or conditions, other than cancer, are generally well-known to those of ordinary skill in the art and will be apparent given the particular disease, disorder, or condition under consideration.

### III. Kits

Also provided are kits for use in the methods as described herein. In some embodiments, a kit comprises a modified Y-shaped adapter as disclosed herein. In some embodiments, a kit comprises a first reagent for end repair to generate end-repaired DNA, wherein the first reagent comprises at least one type of dNTP that comprises a modified base. In some embodiments, the kit further comprises a second reagent for ligating adapters (such as a modified Y-shaped adapter as disclosed herein) to the end-repaired DNA to generate adapted DNA, wherein the second reagent may also seal nicks present in the end-repaired DNA. In some embodiments, the kit further comprises a third reagent for modification-sensitive sequencing that is capable of identifying the base modification in the at least one type of dNTP. The kit may comprise the first, second, and/or third reagents and additional elements as discussed below and/or elsewhere herein. In some embodiments, a kit comprises instructions for performing a method described herein.

Kits may further comprise a plurality of oligonucleotide probes that selectively hybridize to least 5, 6, 7, 8, 9, 10, 20, 30, 40 or all genes selected from the group consisting of ALK, APC, BRAF, CDKN2A, EGFR, ERBB2, FBXW7, KRAS, MYC, NOTCH1, NRAS, PIK3CA, PTEN, RBI, TP53, MET, AR, ABL1, AKT1, ATM, CDH1, CSFIR, CTNNB1, ERBB4, EZH2, FGFR1, FGFR2, FGFR3, FLT3, GNA11, GNAQ, GNAS, HNF1A, HRAS, IDH1, IDH2, JAK2, JAK3, KDR, KIT, MLH1, MPL, NPM1, PDGFRA, PROC, PTPN11, RET, SMAD4, SMARCB1, SMO, SRC, STK11, VHL, TERT, CCND1, CDK4, CDKN2B, RAF1, BRCA1, CCND2, CDK6, NF1, TP53, ARID 1 A, BRCA2, CCNE1, ESR1, RIT1, GATA3, MAP2K1, RHEB, ROS1, ARAF, MAP2K2, NFE2L2, RHOA, and NTRK1. The number genes to which the oligonucleotide probes can selectively hybridize can vary. For example, the number of genes can comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, or 54. The kit can include a container that includes the plurality of oligonucleotide probes and instructions for performing any of the methods described herein.

The oligonucleotide probes can selectively hybridize to exon regions of the genes, e.g., of the at least 5 genes. In some cases, the oligonucleotide probes can selectively hybridize to at least 30 exons of the genes, e.g., of the at least 5 genes. In some cases, the multiple probes can selectively hybridize to each of the at least 30 exons. The probes that hybridize to each exon can have sequences that overlap with at least 1 other probe. In some embodiments, the oligoprobes can selectively hybridize to non-coding regions of genes disclosed herein, for example, intronic regions of the genes. The oligoprobes can also selectively hybridize to regions of genes comprising both exonic and intronic regions of the genes disclosed herein.

Any number of exons can be targeted by the oligonucleotide probes. For example, at least 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 400, 500, 600, 700, 800, 900, 1,000, or more, exons can be targeted.

The kit can comprise at least 4, 5, 6, 7, or 8 different library adaptors having distinct molecular barcodes and identical sample barcodes. The library adaptors may not be sequencing adaptors. For example, the library adaptors do not include flow cell sequences or sequences that permit the formation of hairpin loops for sequencing. The different variations and combinations of molecular barcodes and sample barcodes are described throughout, and are applicable to the kit. Further, in some cases, the adaptors are not sequencing adaptors. Additionally, the adaptors provided with the kit can also comprise sequencing adaptors. A sequencing adaptor can comprise a sequence hybridizing to one or more sequencing primers. A sequencing adaptor can further comprise a sequence hybridizing to a solid support, e.g., a flow cell sequence. For example, a sequencing adaptor can be a flow cell adaptor. The sequencing adaptors can be attached to one or both ends of a polynucleotide fragment. In some cases, the kit can comprise at least 8 different library adaptors having distinct molecular barcodes and identical sample barcodes. The library adaptors may not be sequencing adaptors. The kit can further include a sequencing adaptor having a first sequence that selectively hybridizes to the library adaptors and a second sequence that selectively hybridizes to a flow cell sequence. In another example, a sequencing adaptor can be hairpin shaped. For example, the hairpin shaped adaptor can comprise a complementary double stranded portion and a loop portion, where the double stranded portion can be attached (e.g., ligated) to a double-stranded polynucleotide. Hairpin shaped sequencing adaptors can be attached to both ends of a polynucleotide fragment to generate a circular molecule, which can be sequenced multiple times. A sequencing adaptor can be up to 10, 11, 12, 13, 14, 15, 16, 17, 18, 19,20,21,22,23,24,25,26,27,28,29,30,31,32,33,34,35,36,37,38,39,40,41,42,43,44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more bases from end to end. The sequencing adaptor can comprise 20-30, 20-40, 30-50, 30-60, 40-60, 40-70, 50-60, 50-70, bases from end to end. In a particular example, the sequencing adaptor can comprise 20-30 bases from end to end. In another example, the sequencing adaptor can comprise 50-60 bases from end to end. A sequencing adaptor can comprise one or more barcodes. For example, a sequencing adaptor can comprise a sample barcode. The sample barcode can comprise a pre-determined sequence. The sample barcodes can be used to identify the source of the polynucleotides. The sample barcode can be at least 1, 2, 3, 4, 5, 6, 7, 8, 9, 10, 11, 12, 13, 14, 15, 16, 17, 18, 19, 20, 21, 22, 23, 24, 25, or more (or any length as described throughout) nucleic acid bases, e.g., at least 8 bases. The barcode can be contiguous or non-contiguous sequences, as described above.

The library adaptors can be blunt ended and Y-shaped and can be less than or equal to 40 nucleic acid bases in length. Other variations of the can be found throughout and are applicable to the kit.

All patents, patent applications, websites, other publications or documents, accession numbers and the like cited herein are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number, if applicable. Likewise, if different versions of a publication, website or the like are published at different times, the version most recently published at the effective filing date of the application is meant, unless otherwise indicated.

### EXAMPLES

### Example 1: Methylation Detection in a dsDNA Library Preparation-Based SSM Workflow Using Y-Shaped Adapters Disclosed Herein

A set of samples from healthy subjects and subjects with early-stage colorectal cancer, a type of early-stage cancer (such as lung, breast, prostate, or bladder cancer), or a type of late-stage cancer (such as lung, breast, prostate, or bladder cancer) are analyzed by a blood-based assay to detect differentially methylated regions associated with colorectal cancer. DNA is isolated from a buffy coat sample from the subject and is fragmented. Modified Y-shaped adapters (as disclosed herein, such as shown in FIG. 3, left side) comprising molecular tags are ligated to the DNA such that a sequencing read 1 begins from the 3′ end of an adapter-ligated DNA molecule. The adapter comprises modified cytosines that are resistant to deamination. The DNA molecules are subjected to bisulfite sequencing (or, in other examples, DM-seq or EM-seq), wherein unmodified cytosines are converted to uracils. The modified bases of the adapter are not converted. The DNA is sequenced using NGS (sequencing by synthesis) and one or more base calling metrics (such as clusters passing filter, phasing/pre-phasing, and/or color matrix corrections values) is calibrated based at least in part on data from the resistant region of the Y-shaped adapter ligated to the DNA molecule. The high complexity of the resistant region allows for high quality and high yield sequencing as it is the start of read 1 and is used for cluster quality control (e.g., for the first approximately 25 cycles). At least a portion of nucleobases in a converted region of the DNA molecule are called using the one or more calibrated base calling metrics, resulting in higher quality and higher yield sequencing results as compared to a sequencing by synthesis reaction performed using the same DNA but with current/standard NGS Y-shaped adapters (such as Illumina NGS adapters).

### Example 2: Methylation Detection in a dsDNA Library Preparation-Based SSM Workflow Using End Repair as Disclosed Herein

A set of samples from healthy subjects and subjects with early-stage colorectal cancer, a type of early-stage cancer (such as lung, breast, prostate, or bladder cancer), or a type of late-stage cancer (such as lung, breast, prostate, or bladder cancer) are analyzed by a blood-based assay to detect differentially methylated regions associated with colorectal cancer. DNA is isolated from a buffy coat sample from the subject and is fragmented. The DNA is subjected to end repair using a modified dNTP mix comprising modified cytosines, such as d5mCTP, rather than dCTP, and an A-tailing reaction. The modified cytosines of the dNTP mix are incorporated into the DNA molecule during end repair and are resistant to a conversion procedure. The DNA molecules are subjected to bisulfite sequencing (or, in other examples, DM-seq or EM-seq), wherein unmodified cytosines are converted to uracils. The modified bases incorporated into the DNA molecule during end repair are not converted. The DNA is sequenced using NGS (sequencing by synthesis) and one or more base calling metrics (such as clusters passing filter, phasing/pre-phasing, and/or color matrix corrections values) is calibrated based at least in part on data from the resistant region of the DNA molecule. The high complexity of the resistant region allows for high quality and high yield sequencing because it is at or near the start of read 1 and is therefore used for cluster quality control (e.g., for the first approximately 25 cycles). At least a portion of nucleobases in a converted region of the DNA molecule are called using the one or more calibrated base calling metrics, resulting in higher quality and higher yield sequencing results as compared to a sequencing by synthesis reaction performed using the same DNA but without the use of modified cytosines in the end repair (and without the high complexity region of the end repair ‘scar’ being sequenced at the beginning of read 1).

### Example 3: Methylation Detection in a dsDNA Library Preparation-Based SSM Workflow Using Y-Shaped Adapters and End Repair as Disclosed Herein

The workflow described in this example is illustrated in FIG. 1B. An exemplary standard workflow is illustrated in FIG. 1A for comparison purposes.

A set of samples from healthy subjects and subjects with early-stage colorectal cancer, a type of early-stage cancer (such as lung, breast, prostate, or bladder cancer), or a type of late-stage cancer (such as lung, breast, prostate, or bladder cancer) are analyzed by a blood-based assay to detect differentially methylated regions associated with colorectal cancer. DNA is isolated from a buffy coat sample from the subject and is fragmented. The DNA is subjected to end repair using a modified dNTP mix comprising modified cytosines, such as d5mCTP, rather than dCTP, and an A-tailing reaction. The modified cytosines of the dNTP mix are incorporated into the DNA molecule during end repair and are resistant to a conversion procedure. Modified Y-shaped adapters (as disclosed herein, such as shown in FIG. 3, left side) comprising molecular tags are ligated to the DNA such that a sequencing read 1 begins from the 3′ end of an adapter-ligated DNA molecule. The Y-shaped adapters comprise modified cytosines (such as methylated cytosines) that are resistant to deamination.

The DNA molecules are subjected to bisulfite sequencing (or, in other examples, DM-seq or EM-seq), wherein unmodified cytosines are converted to uracils. The modified cytosines incorporated into the DNA molecule during end repair, and the modified bases of the Y-shaped adapter are not converted. The DNA is sequenced using NGS (sequencing by synthesis) and one or more base calling metrics (such as clusters passing filter, phasing/pre-phasing, and/or color matrix corrections values) is calibrated based at least in part on data from the resistant region of the DNA molecule. The high complexity of the resistant region allows for high quality and high yield sequencing because it is at or near the start of read 1 and is therefore used for cluster quality control (e.g., for the first approximately 25 cycles). At least a portion of nucleobases in a converted region of the DNA molecule are called using the one or more calibrated base calling metrics, resulting in higher quality and higher yield sequencing results as compared to a sequencing by synthesis reaction performed using the same DNA but without the use of modified cytosines in the end repair (and without the high complexity region of the end repair ‘scar’ being sequenced at the beginning of read 1), and/or with current/standard NGS Y-shaped adapters (such as Illumina NGS adapters).

What is claimed is:

1. A method comprising:
(a) performing a sequencing by synthesis reaction on a converted DNA molecule with a sequencing by synthesis instrument, the converted DNA molecule comprising:
ligated adapters;
a converted region comprising one or more nucleobases that have been converted by a conversion procedure; and
a resistant region comprising one or more nucleobases that are resistant to the conversion procedure;
wherein the sequencing by synthesis reaction comprises extending a sequencing primer that binds to the converted DNA molecule upstream of the converted region and the resistant region;
(b) calibrating one or more base calling metrics of the sequencing by synthesis instrument based at least in part on data from the resistant region, thereby providing one or more calibrated base calling metrics; and
(c) calling at least a portion of nucleobases in the converted region using the one or more calibrated base calling metrics.

2. A method comprising:
(a) ligating adapters to a DNA molecule, wherein the adapters comprise a resistant region comprising one or more nucleobases that are resistant to a conversion procedure and the DNA molecule comprises one or more nucleobases that are substrates for the conversion procedure, thereby producing an adapted DNA molecule;
(b) performing the conversion procedure on the adapted DNA molecule, thereby producing a converted DNA molecule comprising a converted region, the converted region comprising one or more nucleobases that have been converted by the conversion procedure;
(c) performing a sequencing by synthesis reaction on the converted DNA molecule with a sequencing by synthesis instrument;
wherein the sequencing by synthesis reaction comprises extending a sequencing primer that binds to the converted DNA molecule upstream of the converted region and the resistant region;
(d) calibrating one or more base calling metrics of the sequencing by synthesis instrument based at least in part on data from the resistant region, thereby providing one or more calibrated base calling metrics; and
(e) calling at least a portion of nucleobases in the converted region using the one or more calibrated base calling metrics.

3. A method comprising:
(a) subjecting a DNA molecule comprising one or more nucleobases that are substrates for a conversion procedure to end repair, wherein the end repair comprises extending a recessed 3′ end of the DNA molecule using a DNA polymerase and deoxyribonucleotides comprising a nucleobase that is resistant to the conversion procedure, thereby generating an end-repaired DNA molecule comprising a resistant region that comprises the nucleobase resistant to the conversion procedure;
(b) ligating adapters to the end-repaired DNA molecule, thereby producing an adapted DNA molecule;
(c) performing the conversion procedure on the adapted DNA molecule, thereby producing a converted DNA molecule comprising a converted region, the converted region comprising one or more nucleobases that have been converted by the conversion procedure;
(d) performing a sequencing by synthesis reaction on the converted DNA molecule with a sequencing by synthesis instrument;
wherein the sequencing by synthesis reaction comprises extending a sequencing primer that binds to the converted DNA molecule upstream of the converted region and the resistant region;
(e) calibrating one or more base calling metrics of the sequencing by synthesis instrument based at least in part on data from the resistant region, thereby providing one or more calibrated base calling metrics; and
(f) calling at least a portion of nucleobases in the converted region using the one or more calibrated base calling metrics.

4. The method of claim 1 or claim 3, wherein the ligating seals one or more nicks present in the end-repaired DNA.

5. The method of the immediately preceding claim, wherein the end repair is performed with a DNA polymerase which does not have 5′-3′ exonuclease activity and/or is not a strand displacing DNA polymerase.

6. The method of the immediately preceding claim, wherein the DNA polymerase is T4 DNA polymerase, T7 DNA polymerase, or Klenow fragment.

7. The method of any one of the preceding claims, wherein the adapter is a Y-shaped adapter that comprises a first strand and a second strand.

8. The method of the immediately preceding claim, wherein
(a) the first strand comprises a first arm region and a first stem region; and
(b) the second strand comprises a second arm region and a second stem region, wherein the second stem region is configured to anneal to the first stem region and the second arm region is configured not to anneal to the first arm region.

9. The method of the immediately preceding claim, wherein the first arm region and second arm region each comprise at least one nucleobase that is resistant to the conversion procedure.

10. The method of claim 8 or claim 9, wherein
(a)(i) the number of nucleobases with a base-pairing specificity complementary to the nucleobases that are resistant to the conversion procedure in the first arm region is greater than the number of nucleobases that are resistant to the conversion procedure in the first arm region, and/or (ii) the number of nucleobases that are resistant to the conversion procedure in the first arm region is less than 25% of the number of nucleobases in the first arm region; and
(b)(i) the number of nucleobases with a base-pairing specificity complementary to the nucleobases that are resistant to the conversion procedure in the second arm region is greater than the number of nucleobases that are resistant to the conversion procedure in the second arm region and/or (ii) the number of nucleobases that are resistant to the conversion procedure in the second arm region is less than 25% of the number of nucleobases in the second arm region.

11. The method of any one of claims 8-10, wherein the first arm region is located 5′ of the first stem region and the second arm region is located 3′ of the second stem region.

12. The method of any one of the preceding claims, wherein the nucleobase that is resistant to the conversion procedure comprises a modified nucleobase.

13. The method of the immediately preceding claim, wherein the modified nucleobase comprises 4-methylcytosine (4mC), 5-methylcytosine (5mC), 5-hydroxymethylcytosine (5hmC), N6-methyladenosine (6 mA), bromodeoxyuridine (BrdU), 8-oxoguanine (8oxoG), 5-pyrrolo cytosine, 5-glucoylhydroxymethylated (5-ghmC), 5-caryboxylcytosine (5-caC), and/or 5-propynyl cytosine.

14. The method of any one of the preceding claims, wherein the nucleobase that is resistant to the conversion procedure is a modified cytosine, optionally wherein the modified cytosine is 5-methylcytosine or 5-hydroxymethylcytosine.

15. The method of any one of claims 8-14, wherein the first arm region, the second arm region, the first stem region, and/or the second stem region comprise one or more modified cytosines, optionally wherein the one or more modified cytosines are 5-methylcytosine, 5-hydroxymethylcytosine, 5-caryboxylcytosine (5-caC), and/or 5-propynyl cytosine.

16. The method of any one of claims 8-15, wherein at least 50%, at least 60%, at least 70%, at least 75%, at least 80%, at least 85%, at least 90%, or at least 95% of the cytosines in the first arm region, the second arm region, the first stem region, and/or the second stem region are modified cytosines.

17. The method of any one of claims 8-16, wherein the first arm region, the second arm region, the first stem region, and/or the second stem region are substantially free of unmodified cytosines.

18. The method of any one of the preceding claims, wherein the resistant region is at least about 10, at least about 11, at least about 12, at least about 13, at least about 14, at least about 15, at least about 16, at least about 17, at least about 18, at least about 19, or at least about 20 nucleotides in length.

19. The method of any one of the preceding claims, wherein the resistant region is about 10-40, about 10-35, about 10-30, about 10-25, about 10-20, about 10-15, about 15-40, about 15-35, about 15-30, about 15-25, about 15-20, about 20-40, about 20-35, about 20-30, or about 20-25 nucleotides in length.

20. The method of any one of the preceding claims, wherein the resistant region is located 3′ of the converted region.

21. The method of any one of the preceding claims, wherein the resistant region comprises at least 2, at least 3, at least 4, at least 5, at least 6, at least 7, at least 8, at least 9, at least 10, at least 11, at least 12, at least 13, at least 14, or at least 15 nucleobases that are resistant to the conversion procedure.

22. The method of any one of the preceding claims, wherein the resistant region comprises 2-30, 2-25, 2-20, 2-15, 2-10, 2-9, 2-8, 2-7, 2-6, 2-5, 2-4, or 2-3 nucleobases that are resistant to the conversion procedure.

23. The method of any one of the preceding claims, wherein the ligation is a sticky-end ligation.

24. The method of any one of the preceding claims, wherein the one or more base calling metrics comprises clusters passing filter, phasing/pre-phasing, and/or color matrix corrections values.

25. The method of any one of the preceding claims, wherein the sequencing by synthesis reaction comprises sequencing the DNA in a manner that distinguishes the first nucleobase from the second nucleobase.

26. The method of any one of the preceding claims, wherein the sequencing by synthesis reaction comprises next generation sequencing.

27. The method of any one of the preceding claims, wherein the sequencing by synthesis reaction comprises generating a plurality of sequencing reads and mapping the plurality of sequencing reads to one or more reference sequences to generate mapped sequence reads.

28. The method of any one of the preceding claims, further comprising determining an epigenetic modification status of at least a portion of the nucleobases of the DNA.

29. The method of any one of the preceding claims, further comprising performing an A-tailing reaction.

30. The method of the immediately preceding claim, wherein the end-repair and the A-tailing reaction are performed in the same reaction mixture, optionally wherein the end-repair and the A-tailing reaction are performed a single tube and/or optionally wherein the end-repair and the A-tailing reaction are performed without an intervening clean-up step.

31. The method of the immediately preceding claim, wherein the A-tailing is performed using a DNA polymerase that does not possess 5′-3′ exonuclease activity and/or is not a strand displacing DNA polymerase, optionally wherein the DNA polymerase is HemoKlen Taq.

32. The method of claim 29 or claim 30, wherein the A-tailing is performed using a thermostable DNA polymerase.

33. The method of claim 20 or claim 30, wherein the A-tailing is performed using Taq DNA polymerase, Tfl DNA Polymerase, Bst DNA Polymerase, Large Fragment or Tth DNA polymerase.

34. The method of claim 29, wherein the end-repair and the A-tailing reaction are performed as separate reactions, wherein a reaction clean-up step is performed after the end-repair and before the A-tailing reaction.

35. The method of claim 34, wherein the reaction clean-up step removes unincorporated dNTPs.

36. The method of claim 34 or claim 35, wherein the A-tailing is performed using a DNA polymerase that does not possess 3′-5′ exonuclease activity, optionally wherein the DNA polymerase is Klenow Fragment lacking 3′-5′ exonuclease activity.

37. The method of claim 30 or claim 34, wherein the A-tailing is performed using a DNA polymerase that possesses 5′-3′ exonuclease activity and/or is a strand displacing DNA polymerase.

38. The method of any one of claims 29-37, wherein the A tailing reaction is performed at a higher temperature than the end repair, optionally wherein the end repair is performed at about 15-35 °C and/or the A tailing is performed at a temperature over about 60 °C, further optionally wherein the temperature over 60 °C is about 60 °C-75 °C.

39. The method of any one of the preceding claims, wherein the conversion procedure comprises deamination of unmodified cytosines of the DNA to uracil.

40. The method of any one of the preceding claims, wherein the conversion procedure comprises contacting the DNA or a subsample thereof with a cytosine deaminase.

41. The method of the immediately preceding claim, wherein the cytosine deaminase is an APOBEC enzyme, optionally wherein the APOBEC enzyme is APOBEC3A.

42. The method of any one of the preceding claims, wherein the conversion procedure comprises enzymatic protection of one or more modified nucleobases of the DNA.

43. The method of the immediately preceding claim, wherein the enzymatic protection comprises glucosylation of the 5-hydroxymethylcytosines of the DNA, optionally wherein the glucosylation comprises contacting the DNA with beta-glucosyltransferase.

44. The method of any one of the preceding claims, wherein the conversion procedure comprises contacting the DNA or a subsample thereof with a ten-eleven translocation (TET) enzyme.

45. The method of any one of the preceding claims, wherein the conversion procedure comprises subjecting the DNA or a subsample thereof to a procedure that affects a first nucleobase of the DNA differently from a second nucleobase of the DNA, wherein the first nucleobase is a modified or unmodified nucleobase, the second nucleobase is a modified or unmodified nucleobase different from the first nucleobase, and the first nucleobase and the second nucleobase have the same base pairing specificity.

46. The method of the immediately preceding claim, wherein the first nucleobase is an unmodified cytosine and the second nucleobase is a modified cytosine.

47. The method of the immediately preceding claim, wherein the modified cytosine is 5-methylcytosine.

48. The method of claim 37, wherein the modified cytosine is 5-hydroxymethylcytosine.

49. The method of any one of claims 45-48, wherein the procedure that affects a first nucleobase of the DNA differently from a second nucleobase of the DNA chemically converts the first or second nucleobase such that the base pairing specificity of the converted nucleobase is altered.

50. The method of any one of claims 45-49, wherein the procedure that affects a first nucleobase of the DNA differently from a second nucleobase of the DNA is methylation-sensitive conversion.

51. The method of the immediately preceding claim, wherein the methylation-sensitive conversion is bisulfite conversion, oxidative bisulfite (Ox-BS) conversion, Tet-assisted bisulfite (TAB) conversion, APOBEC-coupled epigenetic (ACE) conversion, enzymatic methyl-seq (EM-seq), or single-enzyme 5-methylctyosine sequencing (SEM-seq) method.

52. The method of the immediately preceding claim, wherein the Tet-assisted conversion further comprises a substituted borane reducing agent, optionally wherein the substituted borane reducing agent is 2-picoline borane, borane pyridine, tert-butylamine borane, or ammonia borane.

53. The method of any one of the preceding claims, wherein the conversion procedure comprises contacting the DNA with a CpG-specific DNA methyltransferase (MTase) or a CpG-specific carboxymethyltransferase (CxMTase), a methyl donor or a carboxymethyl donor, and a cytosine deaminase.

54. The method of the immediately preceding claim, wherein the cytosine deaminase is an APOBEC enzyme, optionally wherein the APOBEC enzyme is APOBEC3A.

55. The method of any one of the preceding claims, wherein the adapters comprise at least one tag.

56. The method of the immediately preceding claim, wherein the at least one tag comprises a molecular barcode.

57. The method of any one of the preceding claims, wherein the DNA is cell-free DNA.

58. The method of claim 57, wherein the cell-free DNA is in an amount between 1 ng and 500 ng.

59. The method of any one of the preceding claims, wherein the DNA is from a blood sample and/or a tissue sample.

60. The method of claim 59, wherein the blood sample is a whole blood sample, a plasma sample, a buffy coat sample, a leukapheresis sample, or a PBMC sample.

61. The method of any one of the preceding claims, wherein the DNA and/or the sample is from a subject.

62. The method of claim 61, wherein the subject is an animal.

63. The method of claim 61 or claim 62, wherein the subject is a human.

64. The method of any one of claims 59-63, wherein the blood sample is fractionated prior to enriching for at least one epigenetic target region sets of DNA.

65. The method of any one of claims 61-64, wherein the subject has or is at risk of having a cancer.

66. The method of any one of claims 61-65, further comprising determining the presence or status of a cancer in the subject.

67. The method of any one of claims 61-66, further comprising determining the likelihood that the subject has an infection.

68. The method of any one of claims 61-67, further comprising determining the likelihood that the subject has a transplant rejection.

69. A Y-shaped oligonucleotide adapter comprising first and second strands, wherein:
(a) the first strand comprises a first arm region and a first stem region;
(b) the second strand comprises a second arm region and a second stem region, wherein the second stem region is configured to anneal to the first stem region and the second arm region is configured not to anneal to the first arm region;
(c) the first arm region and second arm region each comprise one or more modified nucleobases that are resistant to a conversion procedure; and
(d)(i) the number of nucleobases with a base-pairing specificity complementary to the modified nucleobases that are resistant to the conversion procedure in the first arm region is greater than the number of modified nucleobases that are resistant to the conversion procedure in the first arm region, and/or (ii) the number of modified nucleobases that are resistant to the conversion procedure in the first arm region is less than 25% of the number of nucleobases in the first arm region; and
(e)(i) the number of nucleobases with a base-pairing specificity complementary to the modified nucleobases that are resistant to the conversion procedure in the second arm region is greater than the number of modified nucleobases that are resistant to the conversion procedure in the second arm region and/or (ii) the number of modified nucleobases that are resistant to the conversion procedure in the second arm region is less than 25% of the number of nucleobases in the second arm region.

70. The Y-shaped oligonucleotide adapter of the immediately preceding claim, wherein the modified nucleobase comprises 4-methylcytosine (4mC), 5-methylcytosine (5mC), 5-hydroxymethyl-cytosine (5hmC), N6-methyladenosine (6 mA), bromodeoxyuridine (BrdU), 8-oxoguanine (8oxoG), 5-pyrrolo cytosine, 5-glucoylhydroxymethylated (5-ghmC), 5-caryboxylcytosine (5-caC), and/or 5-propynyl cytosine.

71. The Y-shaped oligonucleotide adapter of claim 69 or claim 70, wherein the nucleobase that is resistant to the conversion procedure is a modified cytosine, optionally wherein the modified cytosine is 5-methylcytosine or 5-hydroxymethylcytosine.

72. A Y-shaped oligonucleotide adapter comprising first and second strands, wherein:
(a) the first strand comprises a first arm region and a first stem region;
(b) the second strand comprises a second arm region and a second stem region, wherein the second stem region is configured to anneal to the first stem region and the second arm region is configured not to anneal to the first arm region;
(c) the first arm region and second arm region each comprise modified cytosines; and
(d)(i) the number of guanines in the first arm region is greater than the number of modified cytosines in the first arm region, and/or (ii) the number of modified cytosines in the first arm region is less than 25% of the number of nucleobases in the first arm region; and
(e)(i) the number of guanines in the second arm region is greater than the number of modified cytosines in the second arm region and/or (ii) the number of modified cytosines in the second arm region is less than 25% of the number of nucleobases in the second arm region.

73. The Y-shaped oligonucleotide adapter of the immediately preceding claim, wherein the modified cytosine comprises 4-methylcytosine (4mC), 5-methylcytosine (5mC), 5-hydroxymethyl-cytosine (5hmC), 5-pyrrolo cytosine, 5-glucoylhydroxymethylated (5-ghmC), 5-caryboxylcytosine (5-caC), and/or 5-propynyl cytosine.

74. The Y-shaped oligonucleotide adapter of any one of claims 71-73, wherein the modified cytosine is 5-methylcytosine.

75. The Y-shaped oligonucleotide adapter of any one of claims 71-73, wherein the modified cytosine is 5-hydroxymethylcytosine.

76. The Y-shaped oligonucleotide adapter of any one of claims 69-75, wherein the first arm region is located 5′ of the first stem region and the second arm region is located 3′ of the second stem region.

77. The Y-shaped oligonucleotide adapter of any one of claims 69-76, wherein the first arm region, the second arm region, the first stem region, and/or the second stem region comprise one or more modified cytosines, optionally wherein the one or more modified cytosines are 5-methylcytosine, 5-hydroxymethylcytosine, 5-caryboxylcytosine (5-caC), and/or 5-propynyl cytosine.

78. The Y-shaped oligonucleotide adapter of any one of claims 69-77, wherein at least 50%, at least 60%, at least 70%, at least 75%, at least 80%, at least 85%, at least 90%, or at least 95% of the cytosines in the first arm region, the second arm region, the first stem region, and/or the second stem region are modified cytosines.

79. The Y-shaped oligonucleotide adapter of any one of claims 69-78, wherein the first arm region, the second arm region, the first stem region, and/or the second stem region are substantially free of unmodified cytosines.

80. The Y-shaped oligonucleotide adapter of any one of the preceding claims, further comprising at least one tag.

81. The Y-shaped oligonucleotide adapter of the immediately preceding claim, wherein the at least one tag comprises a molecular barcode.
